(19) United States
(12) Patent Application Publication
(10) Pub. No.: US 2002/0164824 A1
(43) Pub. Date: Nov. 7, 2002

(54) Method and apparatus based on bundled capillaries for high throughput screening

(76) Inventors: Jianming Xiao, Fremont, CA (US); Shiping Chen, Fremont, CA (US); Yuling Luo, Castro Valley, CA (US); John Butler, San Jose, CA (US); Masoud Qazan, Castro Valley, CA (US); Jochen Thiele, Mountain View, CA (US); Anthony Chen, Sunnyvale, CA (US)

Correspondence Address: Charles D. Holland, Morrison & Foerster LLP, 755 Page Mill Road, Palo Alto, CA, 94304-1018, US

(21) Appl. No.: 10/080,274

(22) Filed: Feb. 19, 2002

Related U.S. Application Data
(63) Continuation-in-part of application No. 09/791,410, filed on Feb. 22, 2001.
(60) Provisional application No. 60/269,642, filed on Feb. 16, 2001.
(60) Provisional application No. 60/271,002, filed on Feb. 22, 2001.
(60) Provisional application No. 60/302,735, filed on Jul. 3, 2001.
(60) Provisional application No. 60/314,747, filed on Aug. 24, 2001.
(60) Provisional application No. 60/315,285, filed on Aug. 27, 2001.
(60) Provisional application No. 60/327,686, filed on Oct. 4, 2001.

(51) Int. Cl. G01N 33/545; C12M 3/00

(52) U.S. Cl. 436/524; 422/102; 422/100; 436/531; 435/287.2; 435/288.4; 216/2

(57) ABSTRACT

Disclosed are systems and methods involved in extreme high throughput screening of compounds which have an affinity for a biological target. The system is based on a capillary bundle with two distinguishable ends wherein capillaries on one end are connected to compounds stored in discrete reservoirs and capillaries on the other end are bound and processed to form a two dimensional microarray. A capillary bundle having reaction wells for hybridization and compound reaction in one end of the capillaries is disclosed. The capillaries may be light-conducting. Also disclosed are various methods of identifying a target compound in a liquid using this capillary bundle as well as methods of fabricating the bundle.

A novel surface tension guided reaction chamber is also provided. Methods and chemistry for fabrication and use of a surface tension guided reaction chamber in binding and hybridization assays are also disclosed. Methods and systems for precise metering of fluids within the capillaries and at the reaction chambers, including the surface tension guided “virtual” reaction well is provided. Methods for performing high throughput screens using optical fiber lined capillaries of the invention are also provided. The capillary arrays are used with both liquid probes as well as probes immobilized on the walls of the capillary.

### CROSS-REFERENCES TO RELATED APPLICATIONS

This application is a continuation-in-part of U.S. application Ser. No. 09/791,410 entitled “Method and Apparatus Based on Bundled Capillaries For High Throughput Screening” by Jianming Xiao et al., filed Feb. 22, 2001 and claims the benefit of priority to the following U.S. Applications: U.S. Application Ser. No. 60/269,642, entitled “Method And Apparatus Based On Bundled Capillaries For High Throughput Screening” by Jianming Xiao et al., filed Feb. 16, 2001; U.S. Application Ser. No. 60/271,002, entitled “Liquid Arrays” by Shiping Chen et al., filed Feb. 22, 2001; U.S. Ser. No. 60/302,735, entitled “Picoliter Fluid Delivery” filed Jul. 3, 2001; U.S. App. Ser. No. 60/314,747, entitled “Desktop Drug Screening System,” filed Aug. 24, 2001; U.S. App. Ser. No. 60/315,285, entitled “Desktop Drug Screening System,” filed Aug. 27, 2001; U.S. Ser. No. 60/327,686, entitled “Single Use XHTS Chip”, filed Oct. 4, 2001; U.S. App. Ser. No. not yet assigned, entitled “Reagent Metering”, by Shiping Chen, filed Feb. 15, 2002, and U.S. application Ser. No. 09/791,411, entitled “Liquid Arrays” by Shiping Chen et al., filed Feb. 22, 2001. All of the above applications are incorporated by reference herein in their entireties as if fully set forth below for all purposes.

### FIELD OF THE INVENTION

The invention relates generally to the field of biochemical analysis in which it is desirable to gauge the interaction of targets from of one or multiple solutions to probes, including the field of high throughput screening (HTS), proteomics, and polymerase chain reaction (PCR) amplification, and the invention provides various methods, devices, and compositions useful in this field.

### BACKGROUND OF THE INVENTION

Many biochemical investigations involve performing a set of experiments that mix one or a small number of reagents with individual chemical or biological entities in a large set and readout the results of the reactions. In “High Throughput Screening” (HTS), the reagents are enzymes and substrates while the entities are a library of chemical compounds. In protein microarray applications, the reagent is the sample protein mixture while the entities are known as protein probes. In polymerase chain reaction (PCR)applications, the reagent is the sample DNA mixture while the entities are pre-designated primers.

In all these applications, it is highly desirable to

Perform as many experiments as possible in parallel; and

Consume as little reagents and biochemical entities as possible in these experiments.

In preferred embodiments, this invention achieves significant enhancement in the above two aspects when compared with existing technologies. HTS is used as an example application to illustrate the functionality of the invention disclosed herein.

The process of drug discovery is critically dependent upon the ability of screening efforts to identify lead compounds with future therapeutic potential. The screening efforts are often described as one of the bottlenecks in the process of drug discovery. One strategy for identifying pharmaceutical lead compounds is to develop an assay that provides appropriate conditions for monitoring the activity of a therapeutic target for a particular disease. This assay is then used to screen large numbers of potential modulators of the therapeutic target in the assay. For example, libraries of chemical compounds can be screened in assays to identify their activity in relation to therapeutic targets and cells.

Biochemical and biological assays are designed to test for activity of chemical entities in a broad range of systems ranging including protein-protein interactions, enzyme catalysis, small molecule-protein binding and other cellular functions. In “High Throughput Screening” (HTS), one uses these kinds of assays to simultaneously test a large number of chemical entities in order to discover biological or biochemical activities of the chemical entities.

Current high-throughput screening (HTS) technologies are based on microtitre plates (96-, 384-, or 1536-well plate) with most widely established techniques utilizing 96-well microtitre plates. In this format, 96 independent tests are performed simultaneously on a single 8 cm×12 cm plastic plate that contains 96 reaction wells. These wells typically require assay volumes that range from 50 to 500 μl. In addition to the plates, many instruments, materials, pipettes, robotics, plate washers and plate readers are commercially available to fit the 96-well format to a wide range of homogeneous and heterogeneous assays.

To date, efforts to improve HTS have generally focused on miniaturization. By reducing the well size the number of wells on each plate is increased in order to provide more parallel testing. Furthermore, by decreasing assay volumes, the cost of reagents is also reduced. Moreover, because more parallel tests can be run with smaller assay volumes, the simultaneous testing of more compounds to find drug candidates is speeded up. Miniaturization has marginally improved the 96-well technology by providing a 384-well format. (Comley et al., J. Biomol. Screening, 2(3):171-78 (1997)).

A system to perform HTS at a higher speed, using less sample volume, and at a lower operational cost is desirable but needs to address the drawbacks of the current formats.

One field in particular where it is desirable to provide a faster rate of HTS is in proteomics. Proteins are the major components of cells. They determine the shape, structure, and function of the cell. Proteins are assembled by 20 different amino acids each with a distinct chemical property. This variety allows for enormous versatility in the chemical and biological properties of different proteins. Human cells have about 100,000 genes for encoding different proteins. Despite the fact that new proteins are being discovered at an unprecedented rate, protein structure and function studies lag behind primarily due to a lack of high throughput methods.

Various DNA chip technologies have been developed to allow the analysis of gene expression in highly parallel fashion. The expression of thousands of genes can be analyzed at one time and this has been tremendously useful in the identification of genes involved in disease processes. Although the expression of a gene in a given cell in general correlates well with its protein expression, it is not always the case. In many instances, protein expression is subject to translational control, which determines if and when predicted gene products are translated. In addition, protein expression is subject to post-translational modification such as phosphorylation. In those instances, the level and activity of proteins within the cells could not be accurately predicted from their nucleic acid sequence or their gene expression pattern. Thus there is a need to study the entire complement of proteins and their expression in normal and disease states.

The current DNA chip technology is difficult to apply to protein arrays because proteins are much more fragile than DNA. Nucleic acid is very robust in nature. It can stand up to heat, can be dried and re-hydrated repeatedly, and can be attached to solid surfaces without loss of activity. In contrast, proteins become denatured and lose their activity with heat, drying, or interaction with non-compatible surface materials. Unlike nucleic acids, such loss of activity of a protein generally occurs when the protein is denatured and subsequently renatured. There is thus a problem with attaching proteins to a substrate, and storing and shipping protein microarrays that are fabricated by traditional nucleic array methods. Maintaining protein activity at solid-liquid interface requires attachment strategies different from those for nucleic acids. Consequently, there is a need to develop a solution based protein array system.

### SUMMARY OF THE INVENTION

The invention provides systems and methods involved in ultra high throughput screening of chemical compounds which have an affinity for or interact with a biological target, as described herein.

This invention provides various new systems and equipment, such as a desktop HTS station or system, a capillary loading station, a capillary array compound library, and combinations and sub-combinations of these three systems. This invention thus includes methods and apparatus for performing HTS operation in a desktop system. It also includes the method and apparatus for the fabrication of such a system. This invention dramatically reduces complexity, cost and at the same time, significantly enhances the throughput in comparison with existing HTS systems.

The HTS system in one aspect of the invention utilizes: (i) a compound loading station; (ii) a capillary array compound library; and (iii) a desktop HTS station. To use a desktop HTS system described in the invention, the compound library is originally stored in mother or daughter plates at a central location. Compounds are first loaded into a miniature capillary array, preferably using the compound loading station. The volumes of compound solutions in each miniature capillary array can vary from tens of microliters to less than a nanoliter depending on the size and configuration of the capillary array and are sufficient for a single or a plurality of screening operations. The compact, capillary array based compound library is distributed to end users, who use the desktop HTS station to conduct high throughput screening operations.

A preferred compound loading station is based on the capillary bundle printing system described in U.S. Ser. No. 09/791,994, 09/791,998, PCT/US01/05695 and PCT/JUS01105844, each filed Feb. 22, 2001, each of which is incorporated by reference in its entirety as if fully put forth below and especially for the disclosure these applications contain on the capillary bundle and methods of making and using the capillary bundle. This capillary bundle printing system is capable of delivering fluid samples from standard micro titer plates to the capillary array compound library (in one instance, a miniature chip) in a highly parallel fashion. The capillary array compound library is an integrated platform for compound storage, compound shipment and assaying. The library provides a means to meter the volume of compounds to be used in the assay and a means to hold the compound with minimum loss during shipment and storage, either in solution or in dried form. Once inserted into the desktop HTS station, the library provides a means to meter the additional reagents used to conduct screening assays. In cases where the compound is shipped dry, it provides a means to re-dissolve the compound into solution. The library also has spaces to mix the reagents with the compound and to hold the mixture for incubation. The library further provides a means to improve signal to noise ratio during readout.

This invention provides a number of different designs for the capillary array compound library, which incorporate different fluid metering, holding and mixing mechanisms, to be described in greater detail later.

The desktop HTS station accepts the capillary array compound library and additional reagents provided by the user. It has fluidic mechanisms to pre-dilute reagents, if necessary, and to deliver reagents to the capillary array holding compounds. It assists mixing reagents with each compound on the array, provides suitable conditions for assay incubation and has an optical system to readout the assay signals.

The invention in one embodiment provides an apparatus for ultra high throughput screening of chemical compounds for a biological target. The system is based on a capillary bundle that has two distinguishable ends, as illustrated in FIG. 1. Capillaries on one end are connected to chemical compounds stored in discrete reservoirs such as microtiter plate wells; capillaries on the other end are tightly bound and then processed to form a two dimensional array that enable the parallel reactions of a target and many different compounds. The apparatus comprises a fluid delivery subsystem, a reaction chamber and a readout subsystem.

The capillary bundle for library compounds delivery can be one as described in pending U.S. patent applications U.S. Serial No. 9/791,944, U.S. Ser. No. 09/791,988, PCT/US01/05695, and PCT/US01/05844, each filed Feb. 22, 2001, which are incorporated by reference in their entirety as if fully put forth herein. In one example of a capillary bundle, 10³-10⁶ capillaries of a few meters long are bundled together, orderly or randomly. The bundle has two distinguishable ends, the unbound end is referred as the input end, the bound end is referred as the output end. Each capillary at the input end is connected to a reservoir, such as a microtitre plate well, that holds a chemical compound in a way that the capillary can draw fluid from the well. The output end of the capillary either contacts or forms the surface of a reaction chamber, as discussed more fully below. The chemical compounds in liquid state are delivered by applying pressure to the reservoirs (as illustrated in FIG. 2) or by gravity (as illustrated in FIG. 3), for instance, or by any of the other methods discussed in pending U.S. patent applications discussed above.

Capillaries may be attached to a reaction chamber. There are a number of reaction chamber designs possible. In one, chemical reactions take place in the tips of the capillaries at their output ends. In another, chemical reactions take place on a through hole plate, such as the one disclosed in U.S. Pat. No. 6,027,873, to Schellenberger et al., which is incorporated by reference in its entirety as if fully set forth herein. These bundled capillaries deliver chemical compounds from a library in the methods described below, although the bundled capillaries may deliver other compounds if desired.

A novel surface tension guided reaction chamber is also provided. Methods and chemistry for fabrication and use of a surface tension guided reaction chamber in binding and hybridization assays are also disclosed. Methods and systems for precise metering of fluids within the capillaries and at the reaction chambers, including the surface tension guided “virtual” reaction well is provided.

Methods for performing high throughput screens using optical fiber lined capillaries of the invention are also provided. Chemical compounds could either be in solution in the capillary or immobilized on the walls of the capillary.

Interaction of the target and chemical compounds can be detected by fluorescence emission (intrinsic or extrinsic probes), fluorescence polarization, luminescence, absorption, surface plasmon resonance (SPR) or other signals of the target system. The detection system can be a CCD based fluorescence imaging system or a scanning based fluorescence system. In the second approach, absorption of samples can also be measured by placing a light source and a detector on different sides of the through hole plate.

While the invention has been discussed above in terms of a capillary array, the invention provides other designs that are not dependent on capillaries. A compound loading station may be a robotic system that is designed to handle mother, daughter, or working microtiter plates to which a liquid delivery system attaches. The liquid delivery system may be, for example, a solid structure with channels or through-holes that fluidly connect the mother or daughter microtiter plates and the compound library. Further, the compound library may be a solid piece that itself has channels or through-holes, and the library may be configured for single use or for multiple uses as described below.

### BRIEF DESCRIPTION OF THE DRAWINGS

FIG. 1 is a perspective view of a capillary bundle in accordance with the present invention.

FIG. 2A illustrates one of the possible configurations of the compound loading station. A pressure chamber containing a compound library in microtiter plates is coupled to capillary bundles. When the pressure chamber is pressurized, compounds in the microtiter plates are delivered to the output ends of the capillary bundle where the assay will be conducted or loaded to another portable capillary array, which will be sent to users who will conduct HTS assay on a desktop screen station, as illustrated in FIG. 2B.

FIG. 3 illustrates another parallel fluid delivery method utilizing gravity as the driving force.

FIG. 4 illustrates one embodiment to fabricate the delivery head.

FIG. 4a illustrates that the capillary tubes are first inserted into through holes of the guiding plate.

FIG. 4b illustrates that a bonding material, such as epoxy or ceramic is used to solidify capillary tubes and the guide plate together.

FIG. 4c illustrates that the solidified bundle is cut at a position very close to the guide plate.

FIG. 4d illustrates that the end facet is polished and etched to form isolated “islands” of the tubes.

FIG. 5 illustrates a number of embodiments of fluidic features on the assay surface to prevent deposited solutions from cross contamination. FIG. 5a illustrates hydrophilic patches on the assay surface. FIG. 5b illustrates geometric structures, such as islands. FIG. 5c illustrates geometric structures, such as wells.

FIG. 6 illustrates the basic configuration of capillary array substrate for the portable compound library.

FIG. 7 illustrates capillary array compound library in different formats. FIG. 7A illustrates a “branch” format. FIG. 7B illustrates a “bundle” format. FIG. 7C illustrates a “chip” format.

FIG. 8 illustrates internal structure of a through hole in a capillary array compound library.

FIG. 9 illustrates a number of structures of the compound storage chamber.

FIG. 10 illustrates a number of internal structures of mixing/reaction chamber.

FIG. 11 illustrates volume metering by surface tension patch.

FIG. 12 illustrates volume metering by a flow regulator with a side air tunnel linking the air above the mixing chamber to the narrow path.

FIG. 13 illustrates chamber volume metering by internal through hole structures with fluid barriers within the chamber.

FIG. 13a illustrates that the barrier may be a short narrow opening.

FIG. 13b illustrates that the barrier may be a short hydrophobic zone.

FIG. 13c illustrates that the barrier may be an interface from a smaller to a larger chamber.

FIG. 14 illustrates process of metering multiple different reagents using multiple interconnected chambers.

FIG. 15 illustrates a through hole structure which comprises multiple chambers linked to a chamber in parallel.

FIG. 16 illustrates a method of excess fluid removal by vacuum.

FIG. 17 illustrates a method of excess fluid removal by a second capillary array.

FIG. 18 illustrates a method of excess fluid removal by wiping.

FIG. 19 illustrates a method for reducing cross-contamination between adjacent holes during excess fluid removal.

FIG. 20 illustrates a reaction chamber design using reflection wall of reaction chamber to enhance optical signal of an assay.

FIG. 21 illustrates another reaction chamber design using light guiding capillary to facilitate optical detection.

FIG. 22A illustrates a method of functionalizing a substrate using protected-aldehyde silanization agent. FIG. 22B illustrates a method of functionalizing a substrate using maleimide silanization agent. FIG. 22C illustrates a method of activating the protected functional groups using light activation.

FIG. 23 illustrates a process for fabrication using a negative mask.

FIG. 24 illustrates a process for fabrication using a positive mask.

FIG. 25 illustrates a typical use of a chambered slide.

FIGS. 26A and 26B illustrate a method of forming a pattern of interspersed regions of differing surface tension using a capillary fiber optic bundle.

FIGS. 27A and 27B illustrate a method of tooling the surface for retaining metered volumes of reagents adjacent an inlet at the assay end of a capillary of an array.

FIG. 28 illustrates a delivery method using an intermediary through-hole array.

FIG. 29 illustrates the operational steps for carrying out an enzymatic assay using a capillary array compound library designed for multiple uses. The through hole structure of the array comprises a micro-reaction well linked to a large compound reservoir through a long and narrow path.

FIG. 30 illustrates the operational steps for carrying out an enzymatic assay using a single use capillary array compound library. The through hole structure of the array comprises a “virtual well” on the assay surface.

FIG. 31 illustrates the operational steps for carrying out an enzymatic assay using a single use capillary array compound library. The through hole structure of the array comprises three interconnected chambers.

FIG. 32 illustrates an embodiment of an assay involving protein arrays or cell arrays.

FIG. 33A illustrates antibody immobilization on the inner wall of a capillary via the carbohydrate moiety. FIG. 33B illustrates antibody immobilization on the inner wall of a capillary via amine groups. FIGS. 33C and 33D illustrates antibody immobilization on the inner wall of a capillary via avidin-biotin or streptavidine-biotin binding. FIG. 33E illustrates antibody immobilization on the inner wall of a capillary via surface attachment, linker formation and thiazolidine formation.

FIG. 34 schematically illustrates a non-equilibrium receptor binding assay within a library formed using fiber optic capillaries.

FIG. 35 schematically illustrates a receptor binding assay within a fiber optic capillary under equilibrium conditions.

FIG. 36 illustrates an enzyme based HTS assay.

FIG. 37 illustrates an example of a cellular HTS assay based on tracking cytosolic calcium ions that can be performed using the system of the invention.

FIG. 38 illustrates an assay for detecting protein-protein interaction using magnetic beads. FIG. 38A illustrates a receptor can be immobilized on magnetic beads. FIG. 38B illustrates a process of allowing the ligand to contact with and potentially react with the receptor by diffusion or by agitation.

FIG. 39 illustrates a capillary array cartridge having multiple chips within it.

FIG. 40 illustrates a metering through hole plate.

### DETAILED DESCRIPTION OF THE INVENTION

The invention provides various HTS systems, components, and associated methods. In one instance, the parallel biochemical assaying system (such as a desktop HTS system) comprises three elements, which are a compound loading station, a capillary array compound library and a screening station. This type of system may be configured at least two ways. In one configuration, the compound library is formed of a capillary bundle having loose ends that can be placed in wells of a microtiter plate, for instance. Opposite ends of the capillaries may be bound together to form the library having optional reaction chambers. In another configuration, a capillary bundle is used to deliver liquids from a microtiter plate to a separate compound library which stores small volumes of the liquids. The compound library also has a reaction chamber at e.g. the end of each capillary or channel of the compound library. In this section, each of the three elements used to form the HTS system will be described in detail. Then, a number of application cases will be presented to illustrate its utility.

Compound Loading Station

This invention provides a compound loading station that delivers compound fluids from a traditional storage medium to a capillary array compound library to be described in the next section.

Currently, almost all compound libraries are stored in standard micro titer plates. Concentrated solutions are in “mother plates” for long-term storage. Periodically, compounds are diluted into “daughter plates” in central compound management facilities. In response to the request from HTS centers, compound solutions may be further diluted into “working plates” and transported to HTS centers in sealed packages.

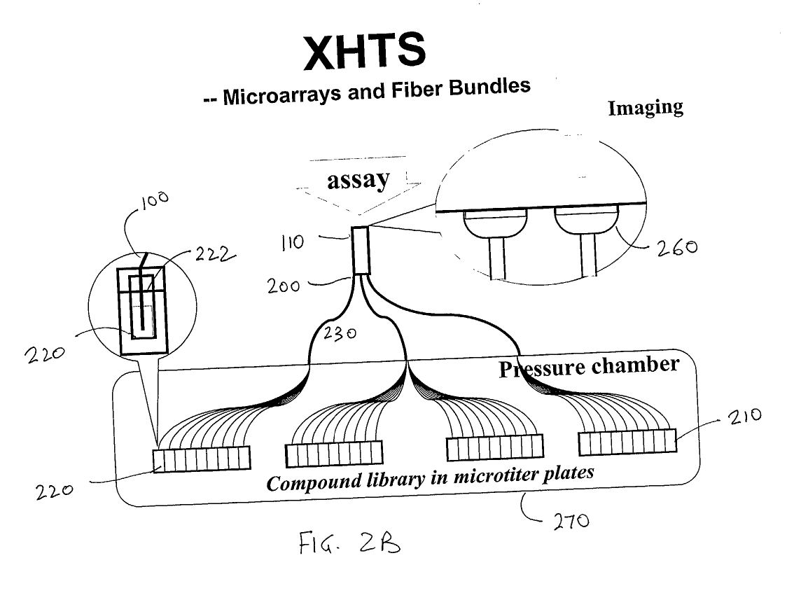

The loading station in this invention provides means to accept and hold microtiter plates, means to accept and hold capillary arrays and means to interface between the two. FIG. 2B illustrates one embodiment of the loading station, where the interface between the micro titer plates and the miniature capillary array is provided by a bundle of capillary tubes that has two distinguishable ends. The capillary bundle for delivering a library of compounds can be designed as described in pending U.S. patent application Ser. Nos. 09/791,944, and 09/791,998. At one end, the tubes are bundled together to form a matrix that is compatible with the array of microscopic reaction sites on the miniature capillary array. At the other end, the tubes are loose, and thus the tubes can be inserted into individual wells of the micro titer plates. The compound fluids are transported from the micro titer plates on to the miniature capillary array in parallel through the tubes.

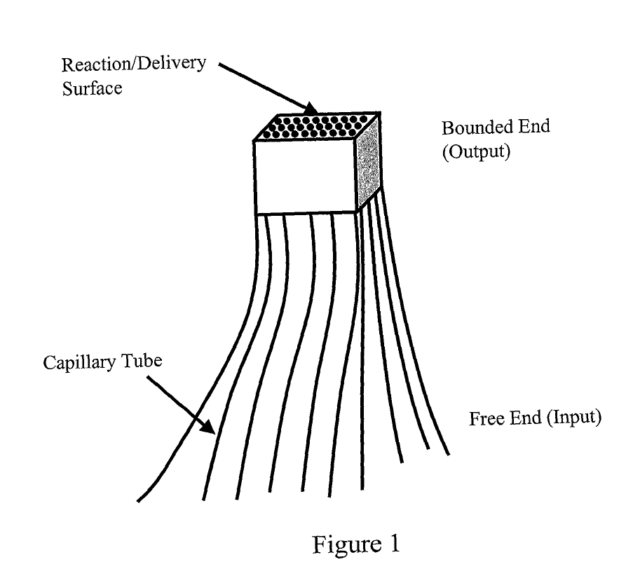

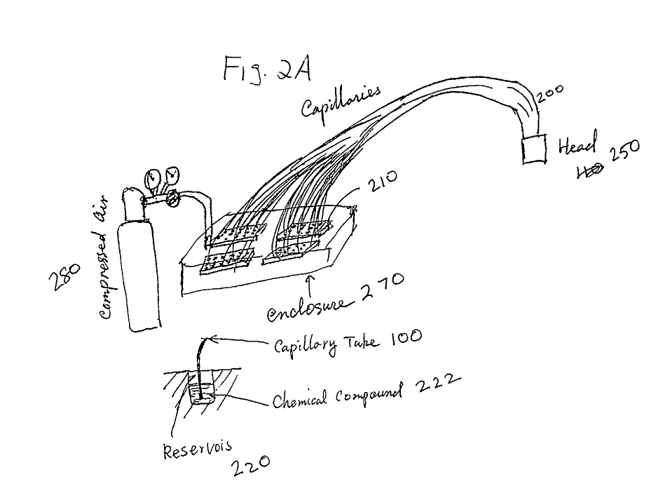

A capillary bundle 110 as depicted in FIG. 1 is fabricated by using capillary tubes, such as those used for capillary electrophoresis. The tubes are bound at one end 102 to form a reaction/delivery head 110. The tubes may be gathered in either a random or an ordered fashion and bound, as discussed in U.S. patent applications discussed above. The minimum number of tubes typically depends upon the number of compounds to be used in a screen. It can be more than 100, preferably more than 10³, more preferably more than 10⁴, more preferably more than 10⁵ or more than 10⁶ or more than 10⁷). The outer diameter of the tubes can range from 5 to 500 micrometers, or preferably 30 to 300 micrometers, or more preferably 40 to 200 micrometers. The inner diameter of the tubes can range from 1 to 400 micrometers, or preferably 5 to 200 micrometers, or more preferably 10 to 100 micrometers. A capillary bundle as described herein may be attached or secured to a frame that is adapted to hold the capillary bundle in a print system. A delivery head may alternatively have a frame that holds a plurality of capillary bundles.

The capillary bundle has two distinguishable ends, the unbound end 204 is referred as the input end, the bound end 202 is referred as the output end. Capillaries on the unbound end 104 may be in contact with a reservoir, such as a microtitre plate well, that holds a chemical compound to be assayed in a way that the capillary can draw fluid from the well. Capillaries on the other end 102 are tightly bound and are typically processed to form a two dimensional array. The minimum number of tubes typically depends upon the number of compounds to be used in a screen (typically 10³-10⁷).

Compound Loading

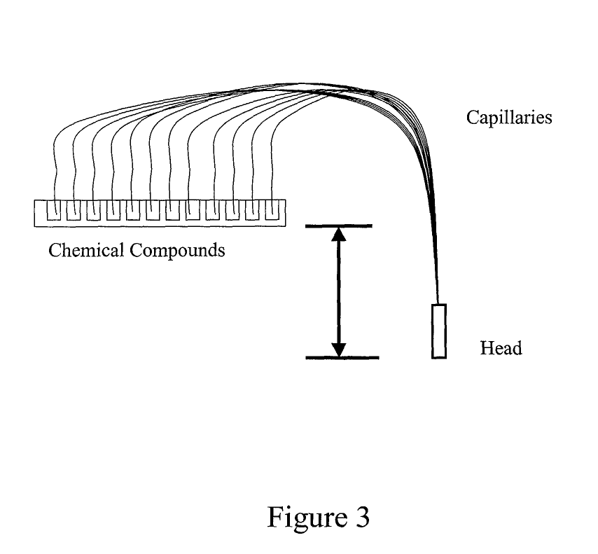

The chemical compounds (including without limitation nucleic acids and their derivatives, lypoproteins, proteins, antigons, antibodies, polysaccharides, lipids, carbohydrates, pharmaceuticals, metabolites, and other organic and inorganic compounds) dispersed in probe fluids are delivered by applying pressure to the reservoirs (as illustrated in FIG. 2A-FIG. 2C) or by gravity (as illustrated in FIG. 3) or by any of the other methods discussed in the pending U.S. and foreign patent applications noted above.

This invention offers several methods to drive fluid from its reservoir into the capillary and towards the reaction chamber. They can be used alone or in any combination of two or more methods in the fluid delivery sub-system. These methods include:

Air pressure A differential air (or other gas such as nitrogen) pressure can be established and maintained between the proximal and distal ends of the capillary bundles, which will translate into hydraulic pressure to drive the probe fluids.

Gravity Once the capillaries are filled with the probe fluids, a constant flow can be maintained and controlled by adjusting the vertical positions of the fluid reservoirs, e.g. the microtiter plates, with respect to the position of the reaction chamber.

Electric field Because fluids are negatively or positively charged, a voltage applied between the reservoir and the reaction chamber can be used to control the flow of the fluid through electrostatic and electro-osmotic force (EOF).

Vacuum The proximal ends of the capillaries may be placed under relative vacuum. The print head and substrate holder may be placed within a vacuum chamber, and the capillaries may extend through a wall of the vacuum chamber and to the reservoirs. The reaction chamber in this instance preferably extends to the wall of the chamber so that thin capillaries are not exposed directly to vacuum if no liquid flows through them.

Pressure Delivery System

FIGS. 2A-2C illustrate an embodiment of a pressure delivery system. One or more microtiter plates 210 are enclosed in a chamber 270. A chemical compound 222 to be assayed is contained within each reservoir or well 220 of the microtiter plate 210. A free end of a capillary tube 100 connects to the well 220 such that it is in contact with the chemical compound 222 which is preferably dispersed in a fluid form. Multiple such capillaries are bundled 230 at an end 200 distal from the chemical compounds 222 to form delivery head 110. In one embodiment, compressed air or an inert gas such as nitrogen 280 is pumped into a sealed chamber 270 carrying the microtiter plates and a chemical compound 222 from a microtiter plate 220 is translated by hydraulic pressure through the capillary tube to the miniature capillary array. In an alternative configuration, the air pressure at the bundled delivery end 200 is made lower than that at the loose end 104, compound solutions are drawn from the reservoirs to the miniature capillary array.

Gravity Delivery System

In a gravity delivery system illustrated in FIG. 3, the chemical compounds 222 are dispersed in the wells of a microtiter plate 320. Capillaries 310 connect at the free end to the microtiter plate 320 and form a reaction/delivery head 300 at the bound end. By positioning the microtiter plate at a height 340 above the head 300, differential gravitational force is used to siphon the chemical compound from the wells of the microtiter plate 320 to the end of the delivery head 300. The height differential may be transiently operated such that once the compound reaches the end of the reaction/delivery head 300 further flow is ceased by eliminating the height differential. Thus the flow of the chemical compound may be controlled merely by altering the height of the microtiter plate 320 relative to the reaction/delivery head 300.

Electric and/or Magnetic Delivery System

A voltage source may be connected to an electrically-conductive material on a facet of the bundled end 102 and to an electrically conductive material contacting the probe-containing liquid near the loose ends of the capillary tubes 104. A voltage regulator may be used to regulate the voltage and thus the rate of deposition of probe molecules.

Another aspect of the invention may have a bundled end, a plurality of reservoirs, and a magnetic field generator that is positioned sufficiently closely to the bundled end to move a magnetic probe-containing fluid (such as a fluid containing magnetic beads or paramagnetic beads having probes optionally attached to their surfaces) through the capillaries of the bundle.

Design and Fabrication of Delivery Head

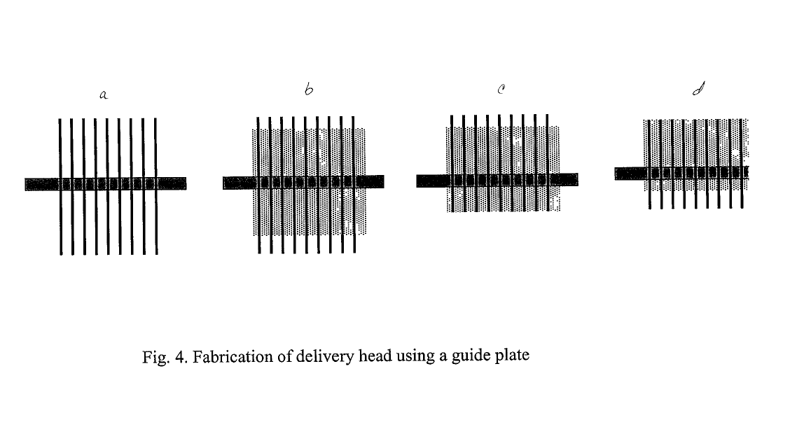

As stated above, the bundled end of the capillary tubes is also termed as delivery head as it directly delivers the compound solution on to the miniature capillary array. This invention provides means at the delivery head to facilitate the delivery. In one embodiment shown in FIG. 4, the delivery head is formed by bonding individual capillary tubes together then cutting and polishing the cut face to form a flat facet. In order to ensure that the compounds are delivered to each assaying site on the capillary array library in parallel without cross-talk, tubes on the facet of the delivery head preferably match the positions of the assaying sites on the capillary array. To ensure this, a guide plate is fabricated which comprises an array of through holes with an exact pattern and pitch as that of the capillary array library. The diameter of the through holes of the guide plate are preferably slightly larger than the outer diameter (OD) of the capillary tubes in the bundle forming the library. FIG. 4 illustrates one embodiment to fabricate the delivery head, where the tubes are first inserted into through holes of the guide plate (FIG. 4a). A bonding material, such as epoxy or ceramic is used to solidify capillary tubes and the guide plate together (FIG. 4b). The solidified bundle is cut at a position very close to the guide plate so that the positions of the tubes are sufficiently close to that of the through holes in the guide plate (FIG. 4c). The end facet is polished and optionally the epoxy or other adherant used to form the solidified mass is etched to form isolated “islands” from the tubes, which prevent fluids in each tube from merging into each other during compound loading (FIG. 4d).

Features of Receiving Capillary Array Compound Library

Fluidic features are built into the capillary array compound library that receives and stores the compound fluids. These features will be presented briefly here and in detail in the next section.

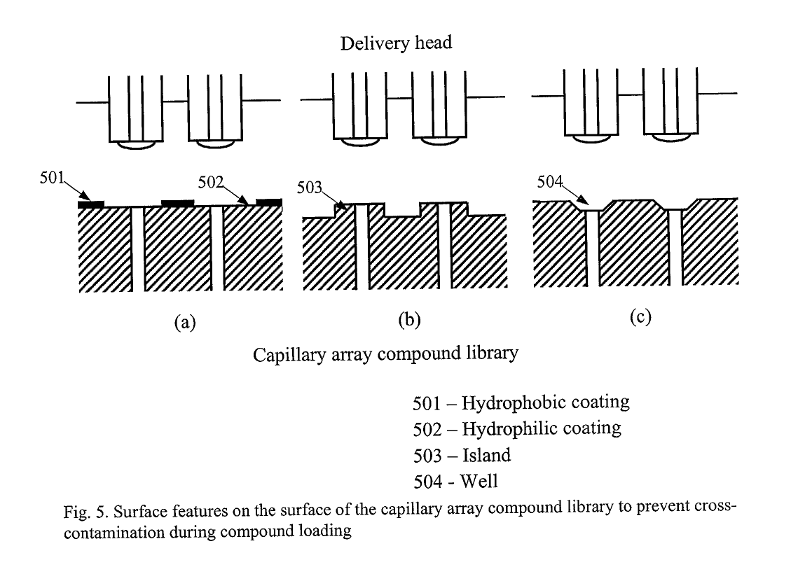

The capillary array compound library comprises a substrate having a large number of assaying sites that terminate at a common surface, which is termed the “assay surface”. The assaying sites may be through-holes that pass through the substrate and may have the same cross-sectional area from one end of the substrate to the other. Alternatively, the assaying sites may be reaction chambers that have a larger cross-sectional area than the through-holes mentioned above. Each assaying site comprises at least one chamber capable of storing compound solutions, which may be the through-hole discussed above or a portion of the through-hole. The compound loading station deposits the compound solutions on the assay surface and the solutions are drawn into different compound storage chambers by capillary force or pressure. Fluidic features may be formed on the assay surface to isolate deposited solutions so that they will not merge into each other causing cross contamination or fluid “cross-talk”. A number of embodiments of these features are illustrated in FIG. 5, which include hydrophilic patches (FIG. 5a) or geometric structures, such as, islands (5b) or wells (5c), that optionally mate with the delivering capillaries from the loading station.

Capillary Array Compound Library

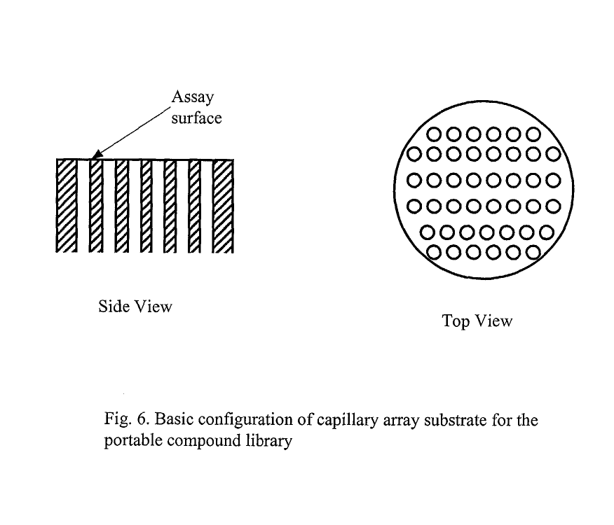

In one embodiment of the invention, the capillary array compound library is a portable medium that provides the means to facilitate compound storage, reagent metering, mixing and readout. As illustrated in FIG. 6, the basic configuration of the capillary array compound library comprises an array of assaying sites that are in fluidic connection to a common surface, which is termed the “assay surface”. Each assaying site may have at least one inner space capable of storing a compound and at least one other space for mixing reagents. The compound storage chamber is in fluidic connection with the reagent mixing space, and both are connected to the assay surface. Different compounds are held within the individual compound storage spaces. Additional reagents that are common to all assay sites are introduced to the assay surface and drawn into each assaying site which may have built-in fluidic features to perform or assist additional assaying functions such as volume metering, mixing and readout.

The number of assaying sites in the array directly relates to the number of screening assays to be performed in parallel, which is preferably more than 100, preferably more than 500, more than 1000, more than 5000, more preferably more than 10,000, more than 100,000 or more than 1,000,000. One or multiple such capillary arrays may be used to hold an entire compound library. The assaying sites are grouped on the assay surface at a spatial density of at least 40 per square centimeter, preferably more than 200 per square centimeter, more preferably more than 400 per square centimeter, more preferably more than 1,000 per square centimeter, or more than 4,000 per square centimeter, more preferably more than 10,000 per square centimeter, more than 40,000 per square centimeter, or more than 100,000 per square centimeter. The compound storage space at each assay site preferably holds fluid at a volume of no more than 100 microliters, preferably no more than 10 microliters, more preferably no more than 1 microliter, more preferably still no more than 100 nanoliters, preferably no more than 10 nanoliters, more preferably no more than 1 nanoliter, more preferably no more than 100 picoliters, more preferably no more than 10 picoliters, and more preferably still no more than 1 picoliter. The reagent mixing space preferably has a fluid holding capacity of no more than 10 microliters, preferably no more than 1 microliter, more preferably no more than 100 nanoliters, more preferably no more than 10 nanoliters, more preferably no more than 1 nanoliter, and more preferably still no more than 100 picoliters. For singleuse compound libraries, the volume ratio of reagent mixing space over compound storage space is preferably greater than 10, preferably greater than 50, more preferably greater than 100 for HTS applications such as enzymatic assays. This volume ratio may be greater than 100, preferably greater than 500, and more preferably greater than 1,000 for HTS applications such as cell based assays. For other applications, such as a protein array or PCR, this volume ratio can be as small as 5 or even 2. It is desirable, for compound storage space in particular, to have a large volume (cubic micron) to exit opening (micron) ratio in order to reduce evaporation and potential contamination. In order to define this ratio as a pure number, the “volume” of an opening in this context is defined as the total volume of one or multiple largest possible spheres that can pass through the opening simultaneously. That is, the “volume” of an opening is the volume of a single sphere that can fit into the opening. If the channel has a circular cross-sectional area, the diameter of the sphere is equal to the diameter of the channel. The volume to opening ratio of the compound storage space is at least 2, preferably at least 10, more preferably at least 40, or more preferably at least 100, or at least 200. The volume to opening ratio of the reagent mixing space is at least 1, preferably at least 2, more preferably at least 5, or at least 10. The length to diameter ratio (“aspect ratio”) of the compound storage space is preferably no less than 10, 20, 50, or more preferably no less than 100, 200, or 500.

In one particular embodiment, an assay site comprises at least one hole substantially perpendicular to the assay surface. The internal structure of the hole comprises multiple interconnected chambers or wells or a combination of wells and chambers. In a further specific embodiment of the hole configuration, the assay hole is a through hole that has a second exit that may be on the same assay surface or on a second surface that is substantial parallel to the assay surface. The capillary array may be made of any suitable material such as glass, silicon, polymer, ceramic or suitable metal.

Formats

The capillary array compound library can take a number of physical formats. The formats described in this section are for illustrative purposes only and not exhaustive, and one skilled in the art may fabricate any number of configurations which are within the invention as described herein.

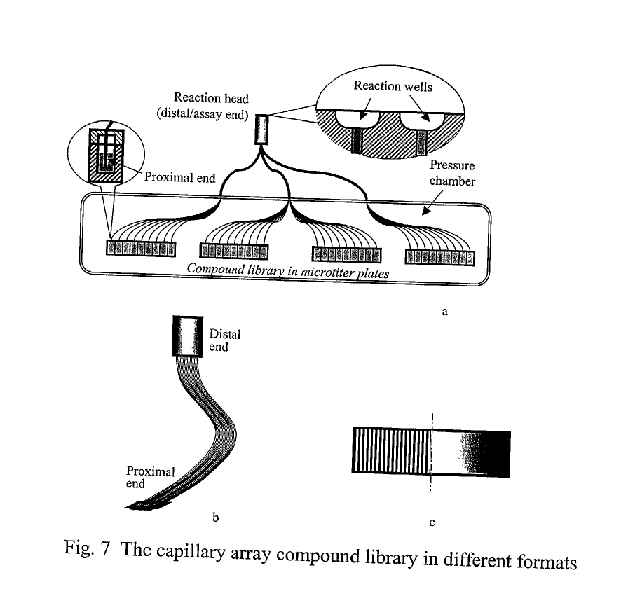

In a first configuration, referred to as a “branch” format, as shown in FIG. 7A, through holes are the channels of individual capillaries. The length of the capillary can range from about 100 meters to about 0.5 meter and the outer diameter of the capillary can range from about 2 mm to about 10 μm. For each capillary, a proximal end is inserted into a liquid reservoir (such as a well in a standard microtiter plate) while the distal end is bundled together with that of many other capillaries and formed into a solidified piece. In short, the capillary tube bundle in the loading station presented above is used directly for assaying. Additional features can be fabricated on the facet of bundled ends to facilitate reagent metering and mixing, as described in later sections.

A second configuration is referred to as a “bundle” format, as shown in FIG. 7B. The through holes are channels of individual capillaries which have outer diameters of about 2 mm to about 10 μm for instance. A large number of capillaries are bundled along the entire length from a proximal loading end to a distal reaction head end, either loosely or as a solidified unit. The diameter of the channel in the capillary is sufficiently small and the inner surface of the channel is sufficiently hydrophilic that liquid probes are retained within the channel by capillary force. The length of a bundle can range from about 0.1 m to hundreds of meters.

The array in bundle format can be fabricated directly from an array in branch format after individual liquid probes are pumped into the capillaries. The loose end of each capillary in the array can be taken out of the probe reservoir that it is inserted into and grouped together to form a capillary bundle that is bundled along its entire length. Liquid probes are stored within the cavities of capillaries and the stored volume is determined by the length of the capillary bundle and the inner diameter of the cavity. For example, a bundle of 1 m in length with a cavity diameter of 20 μm can store 0.3 μl probe liquid, sufficient for hundreds of experiments.

In a third configuration, referred to as a “chip” format, as shown in FIG. 7C, all through holes are formed in a solid piece, which takes a chip shape having a top 680 and a bottom 690 surface where probe liquids may enter and exit the through holes. Similar to the previously described formats, the diameters of the holes are sufficiently small and inner surfaces of the holes are sufficiently hydrophilic such that liquid probes are retained within the channel by capillary force. The thickness of the chip 692, and hence the length of the through holes, can range from about 50 μm to several tens of centimeters, preferably ranging from 200 μm to 1 centimeter, more preferably 500 μm to 2 millimeters. The size of a chip can be as small as 1 mm×1 mm, as large as 130 mm×130 mm. The through hole pattern can be randomly or orderly distributed. In the case of orderly distributed hole pattern, the hole pattern matches that of the delivery head capillary, or, in another example, matches the well pattern of a microtiter plate (96, 384, 1536, 3072, or 6144 well). A chip with microtiter plate pattern can be used as a “compound library cover” for a microtiter plate. The size of the chip can range from 5,000 cm² to 0.01 cm², or preferably from 1,000 cm² to 0.01 cm², or more preferably from 100 cm² to 1 cm². The array of assaying sites on the assaying surface has a spatial pitch ranging from 10 mm to 1 μm, or preferably 1 mm to 10 μm, more preferably 500 μm to 50 μm. The cross-section of the through hole may be circular or any other shape. Further, it may have the same shape and dimension along its length, or more preferably, it is structured to provide additional assaying functions as described in detail later. The through hole structure may have branches or junctions that involve multiple paths. In most cases, the through hole has its second opening on a second surface that is substantially parallel to the first surface, where the first opening of the through hole exits. It is also possible that the second opening of the through hole exits on the same surface as the first one. The diameter of the through hole ranges from 10 mm to 0.1 μm, or preferably from 1 mm to 1 μm, more preferably from 400 μm to 10 μm.

The capillary array chip can be fabricated in many different ways. It may be assembled from bundling ready-made individual long capillary tubes through out the entire length. The bundling can be achieved through epoxy or fusion bonding, for instance. The long bundle is then cut to a desired length. This method may be used to make a capillary bundle that has a hole pattern identical to the hole pattern of the capillary array chip. A capillary array bundle formed from an ordered array of capillaries is fused along its length such that multiple chips can be cut from the fused portion of the bundle. Once a number of chips are cut, a fused portion remains attached to the bundle and is is used for fluid delivery to the chips made from the bundle. This assures that the through hole pattern in the face of the capillary bundle is identical to the hole pattern in the chips cut from the bundle.

A second way to form a capillary chip is to bundle large preform tubes together and extrude the preform bundle into a long solid capillary bundle, then cut the bundle to form chips of desired length(s). A third way to form a capillary chip is to mold a large preform having an array of through holes using a suitable powder mixture, usually made of a ceramic or metal oxide. The powder is solidified through heat fusion, then extruded to reduce to the capillary pitch and finally cut to desired length(s) to form the capillary chip(s). The fourth way of forming a capillary chip is to start with a solid chip substrate made of silicon, glass, plastic, ceramics, metal oxide, metal or other suitable materials. Through holes are fabricated in the substrate using available micromachining technologies, which are widely used for microelectromechanical systems (MEMS) applications and include etching, especially deep reactive ion etch (DRIE), laser drilling, mechanical drilling, ultrasonic drilling, sand blast drilling, micro-molding, LIGA (lithography, electroforming, and molding), electric plating and wafer bonding. One additional way to form a capillary array chip used to form a capillary array compound library is to form individual features in separate slides or substrates, the join or fuse the separate pieces together to form the chip. For example, a reaction chamber may be formed in silicon substrate #1 by etching the substrate using MEMS fabrication technology, capillary through holes may be formed in two separate silicon subsgtrates #2 and #3 by etching them, narrower channels that act as flow restrictors between capilllary through holes may be formed in silicon substrate #4, and the substrates may be stacked in the order substrate #1/substrate #2/substrate #4/and substrate #3 and then fusion bonded together in an oven to form the capillary array chip or capillary array compound library.

Most fabrication methods for chip format capillary array are to make a chip substrate with empty through holes first, then use a dedicated loading station as described above, for example, to load compound solution into the holes. In an alternative method, a compound solution is first loaded into the channel of a very long, stand-alone capillary by pressure. Then the solution can be dried in the capillary. Alternatively, the capillary can also be frozen to fix the compound in place in the capillary. Next, many such capillaries filled with different compounds are bundled together using various bonding methods including gluing, diffusion bonding, soldering, or other method known to one of ordinary skill. Finally, the very long bundle can be cut to length as required. The cutting can be carried out using various devices, which include a diamond saw (wire and disk), laser, water jet, plasma beam and other known cutting system.

Internal Structures of Through Holes and Their Functions

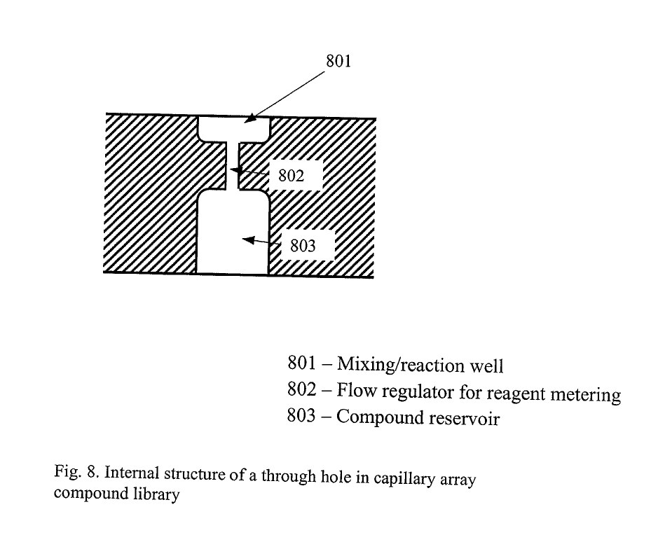

Preferably a capillary array compound library comprises an array of through holes. Each through hole may provide a means to store, meter and mix reagents used for the assay and to assist readout results. FIG. 8 illustrates one embodiment of the internal structure of a through hole. This is a typical structure which generally comprises a reservoir for compound storage, a chamber for reagent mixing and reaction, and additional features on the assay surface that localize liquid to particular areas to prevent cross-contamination during compound and reagent loading. Other functions may also be integrated in the through hole structure which enables precision metering of reagents, reduces evaporation and assists optical detection, respectively. These structural features and their functions are described in detail below.

Compound Storage Chamber

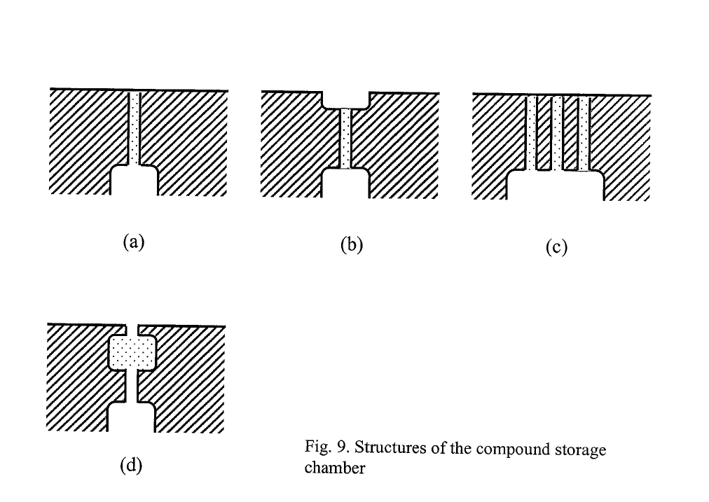

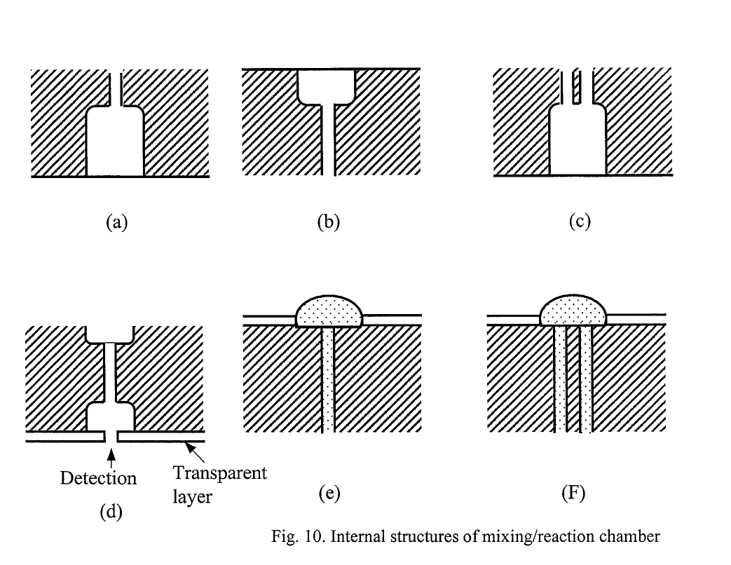

Compound storage in a miniature and portable form is the basic function of the capillary array compound library. Preferably, each compound is in pure DMSO (dimethylsulfoxide) solution or other polar solvent and is stored in a chamber along the through hole (FIG. 9a, b), or, in some cases, in multiple through holes (FIG. 9c). The through hole structure is ideally suited to store solutions in very small volumes as called for in HTS applications. As the evaporation rate is directly proportional to the surface area exposed to air, evaporation can be minimized by using tubes with small diameters or small openings for compound storage (FIG. 9d). Evaporation may further be minimized by sealing or covering the capillary ends using e.g. a polymer or metallic film adhered to the edges of the surface of the substrate. Preferably, the film is hydrophobic to prevent the film from removing any liquid from a through hole when the film is removed. Inert gas may be used to extend compound storage shelf life as well.

The inner volume of the storage chamber can be designed to hold sufficient compound volume for single or multiple uses. In certain applications, the compound solution may be dried after the compound is loaded into through holes using the loading station. The dried compound powder will reside inside the storage chamber, preferably attached to the inner wall.

A re-dissolving stage is carried out using pure DMSO or other polar solvent in the screening station after the capillary array compound library is shipped to the users. This will be discussed in a later section.

Reagent Mixing/Reaction Chamber

In a typical enzymatic assay concerned with HTS, reagents include three different solutions, i.e. compound, enzyme and substrate. These reagents have to be mixed thoroughly and incubate for a certain period of time. The invented library provides a structure for the mixing of reagents required in an assay. This structure can be a chamber in the through hole, which is similar to the compound storage chamber but usually much larger in volume and dimensions, as shown in FIGS. 10a and 10b. The mixing chamber may link to multiple parallel chambers to receive different reagents (10c). A cover with a very small opening is integrated to the mixing/reaction chamber in the design shown in FIG. 10d to reduce evaporation during incubation. This cover is preferably transparent to allow optical reading through the cover.

A well on chamber as discussed herein may be either a physical well, such as a depression in a surface, or a virtual well. FIGS. 10e, f show an alternative “virtual well” design for the mixing/reaction chamber, which comprises a hydrophilic patch around the entrance of a through hole. The patch is surrounded by a hydrophobic region. Fluid can be held within the boundary of the patch by surface tension force.

The invention may also provide structural features that enhance mixing. As illustrated in FIGS. 10a to 10f, the mixing chamber has a much larger cross-section in comparison with that of the path between the reagent reservoir and the mixing/reaction chamber. A micro vortex can be generated when the reagent flows into the mixing chamber, which greatly enhances mixing, by moving the fluid rapidly through the capillary and into the reaction chamber. Additional microfluid features can be built at the entrance to the mixing chamber to further enhance the mixing. These include microcomb or micro-hive structures that split flow into many branches resulting in enhanced diffusion and creation of micro-vortexes.

Metering

In most modern scientific studies involving biological or chemical reactions, volumes of the reagent fluids that take part in the reaction have to be very precise in order to obtain the desired result. Currently, control of reagent volume is usually achieved by using a dedicated device that meters the fluid volume and then dispenses the measured volume into a container for mixing and reaction.

There is a great need in reducing the volume of reagent fluids used in a screening experiment. This is especially true in the area of high throughput screening (HTS). This is because most compounds used in HTS are expensive and some are precious and purified from rare natural substances. In addition, preparing a large quantity of sample is time consuming, costly and sometimes impossible. Currently, a state-of-the-art HTS system requires dispensing volumes of compounds on the order of tens of nanoliters with standard deviation (“CV”) less than 10%. Such a requirement for precision is very challenging using existing metering and dispensing technologies. At such a small volume, the amount of residual fluid left on the tip of the dispenser becomes a significant factor affecting the dispensing CV as it is very difficult to eliminate the residual or make the residual volume consistent. This has become a major bottleneck in further reducing the reagent consumption.

There are a number of ways to meter reagent volume on the destination container. One method is to dispense an approximate amount of required volume, then precisely measure the actual volume using visual or other means and eventually factoring the actual volume into the final result mathematically. This method requires direct measurement and mathematical manipulation of data to derive the information desired.

This portion of the invention provides a novel concept that integrates containing, metering and mixing functionalities into a single platform, which reduces or eliminates the fluid volume error caused by reagent dispensing as described above.

The reason that dispensing becomes a major source of volume error is due to the fact that reagents are dispensed AFTER they are METERED in dedicated equipment. This invention proposes to eliminate such an error source by a completely new approach: metering at the destination after DISPENSING the reagent. This method and system are applicable to many microfluidic systems in which accurate droplet metering is required.

One specific embodiment of this invention is to design the destination container so that it not only is used as a container for reagent mixing and reaction but also facilitates additional functions such as reagent metering and readout. In this design, excess reagent is dispensed to the assay surface of the library or destination container, then geometric or other fluidic constraints retain a desired volume on the surface or in a designated chamber. Excess fluid is then removed from the destination container. Three different embodiments of this configuration are presented below:

Metering with Surface Tension Patch

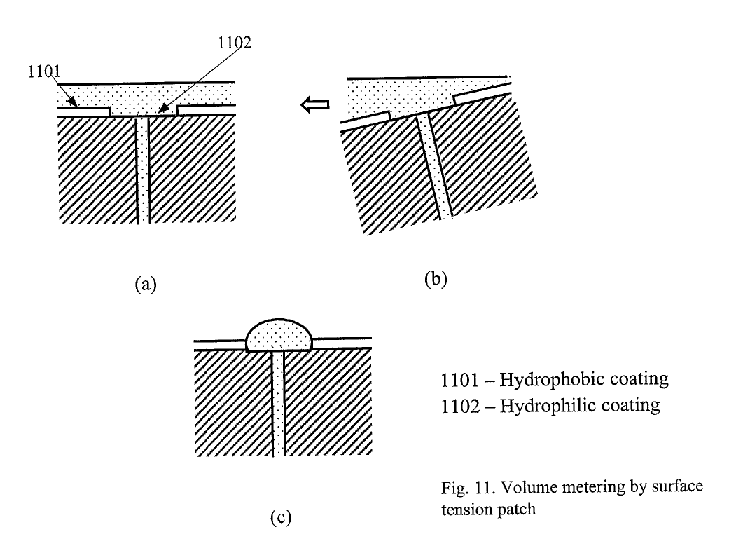

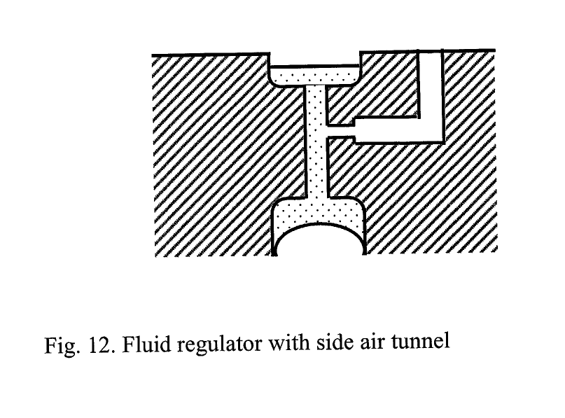

As illustrated in FIG. 11, a hydrophilic patch 1102 is surrounded by a hydrophobic area 1101. As described in the above section, this configuration forms a “virtual well” and is capable of holding a certain amount of fluid. The fluid volume that can be held by the patch is determined by the size of the patch and fluid contact angle of the hydrophobic area surrounding the patch (11c). Metering is achieved by applying abundant fluid to the patch (11a) and removing the excess fluid by various methods, which include tilting the surface at an angle sufficient to allow excess reagent to run off the surface (11b), centrifuging or applying a vacuum of a suitable strength (11b).

In this instance, the liquid to be dispensed at through holes of a library has a surface tension that produces a droplet of given volume in the hydrophilic region in which the droplet forms. The surface of the droplet at the hydrophobic/hydrophilic interface on the surface of the library has a certain contact angle that depends on the surface tension of the liquid being dispensed. The size of the droplet is thus a function of the size of the hydrophilic area (if the liquid being dispensed is polar) and the contact angle of the droplet's surface. Thus, there are two ways to meter appropriate volumes of liquid. One is to provide a hydrophilic patch of desired size for a liquid of given surface tension, and the other is to adjust the surface tension of the liquid to form a droplet of the desired volume in a hydrophilic patch having a given or known size. Surface tension of liquids may be adjusted by means known to those of ordinary skill in the art, and these include adding salts such as sodium chloride and potassium chloride, and detergents such as sodium dodecylsulfate (SDS), sodium lauryl sulfate, and sodium laureth sulfate (SLS).

Another design of this invention involves creating microgrooves circling or otherwise surrounding the immediate opening of through holes. Such microgrooves retain solution by capillary action. Preferably, the width of the grooves is no more than 100 microns, more preferably no more than 20 microns, and even more preferably no more than 10 microns. Preferably, the depth of the grooves is no more than 50 microns, more preferably no more than 15 microns, and even more preferably no more than 5 microns.

Metering with a Flow Regulator

In one embodiment of this invention, a flow regulator is provided between the mixing/reaction chamber and the reagent reservoir. The volume of reagent delivered to the mixing chamber can be controlled by external fluid pressure and its application duration. This regulator can be simply one or multiple narrow paths linking a mixing chamber and the reagent reservoir, as shown in FIG. 8. The narrower the path, the more control there is over the flow. In the structure shown in FIG. 8, the mixing/reaction chamber maintains a fluid connection with the reagent reservoir. FIG. 12 shows a more sophisticated regulator structure, which provides a side air tunnel linking the air above the mixing chamber to the narrow path. At the end of reagent delivery, the pressure on the reservoir side will decrease and draw in air from the side tunnel, which forms an air bubble to isolate the reagent in the reservoir from the fluid in the mixing chamber.

Metering with Through Holes

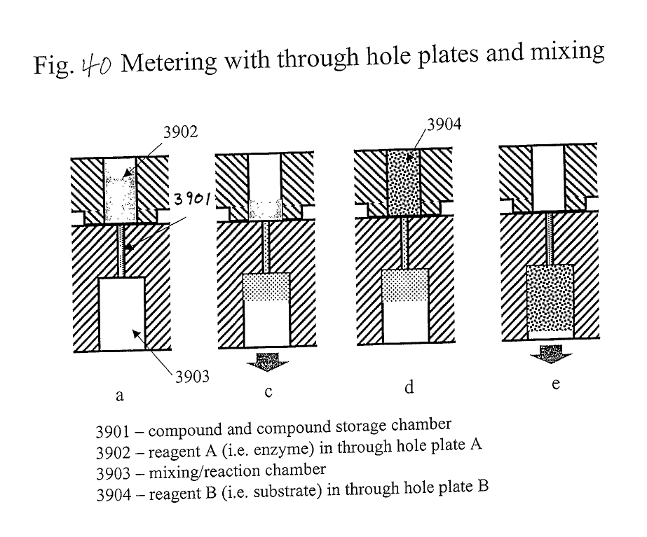

This invention also uses the inner space of a through hole to meter reagents. The inner surface of the through hole is preferably made hydrophilic. When a fluid is present at the entrance of the hole, the capillary force will thus draw fluid into the hole. If an excessive amount of fluid is present, the entire inner space of the hole will be filled. By removing the rest of the fluid outside the hole, the fluid volume is metered to be equal to the volume of the inner space of the through hole. Different reagents can be metered with separate through hole plates. To mix these reagents, through holes on different substrates can be aligned to establish a fluid link to a larger mixing chamber. Pressure will be provided to drive fluids through connecting through holes into the mixing chamber. This is illustrated in detail in FIG. 40. Preferably, the diameter of the through hole for holding reagents is 50% or more larger than the diameter of the compound storage capillary, more preferably 100% or more larger, and even more preferably 300% or more larger. Preferably, the ratio of the space of each through hole in the reagent plate to the space for holding each compound in the capillary compound library is more than 10, more than 50, more than 100, or more than 1000. Proper level of compound dilution can be achieved with such ratios.

Metering with Interconnected Chambers

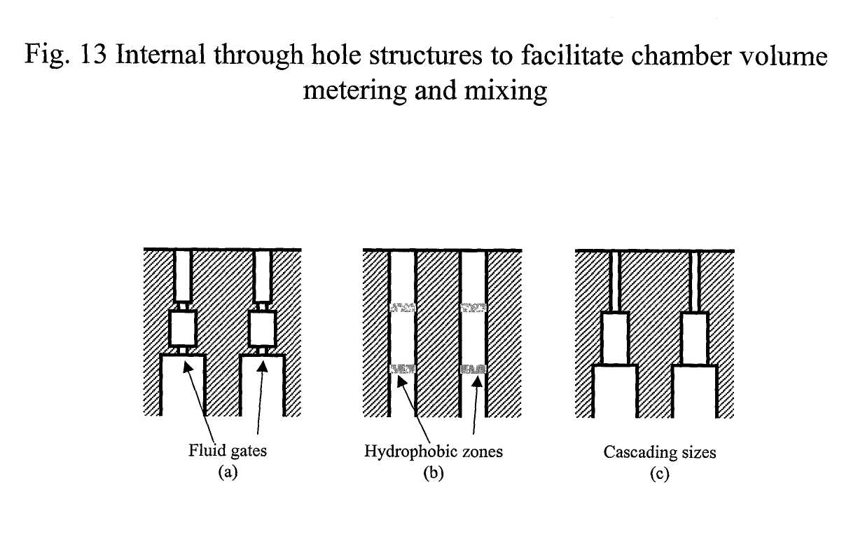

This embodiment of the invention uses a chamber in the through hole to meter reagents. The inner surface of the chamber is made hydrophilic and is separated from other portions of the through hole by a fluid barrier, which prevents fluid from crossing when the pressure differential is less than a certain “bursting pressure”. Such barrier may be a short narrow opening as shown in FIG. 13a or a short hydrophobic zone (13b). Alternatively, it can be an interface from a smaller to a larger chamber (13c) or a combination of any of these. One method for constructing such internal structure is to build each chamber on a separate wafer using existing micro-fabrication methods such as deep reactive ion etching, micro molding, electro plating or chemical vapor deposition and then bonding the multiple wafers together. The chamber has a sufficiently small cross-sectional area that fluid is drawn into the chamber by capillary force when fluid is present at the entrance to the chamber. When the fluid fills the chamber to the fluid barrier, capillary force prevents the fluid from breaching the barrier, thus confining a definite amount of fluid to the chamber. After the chamber is filled, excess fluid can be removed from the top surface of the substrate by one or a combination of the following means: 1) blotting, 2) drawing excess fluid from the surface using a vacuum pressure that is less than the “bursting pressure”, 3) capillary force using another dry capillary array placed on the wetted surface of the first capillary array to draw excess fluid from the surface of the first capillary array using capillary force; 4) wiping, and 5) air knife blowing. In method 4, the pore size or pore cross-sectional area of the dry capillary array that is used to remove the excess liquid should in general be larger than the pore size or pore cross-sectional area of the capillaries in the first array in order to avoid withdrawing liquid from the designated reagent chamber of the first array. In this way, liquid inside the through hole of the first capillary array will not be removed.

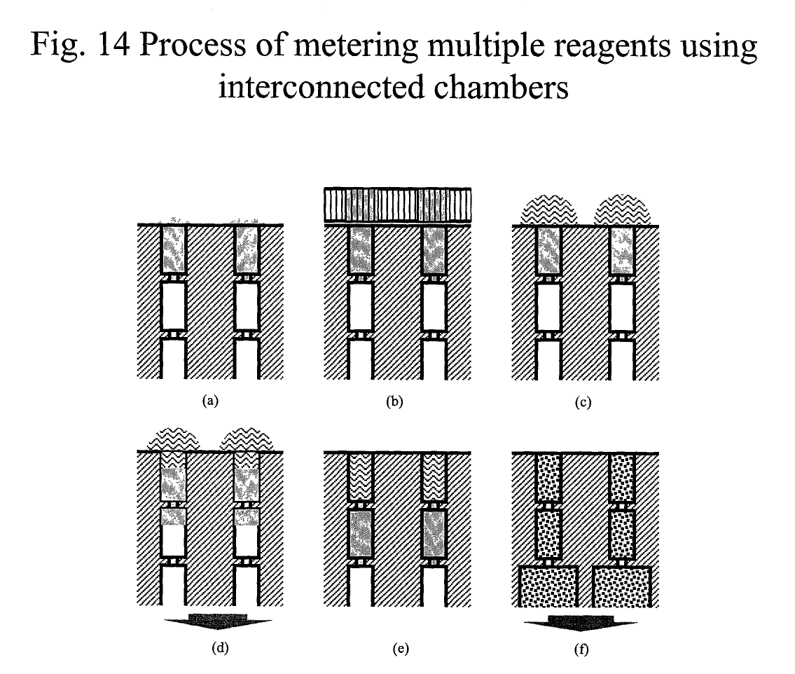

If an assay requires the mixing of multiple different reagents, multiple interconnected chambers can be used to meter every reagent, as illustrated in FIG. 14. The first reagent applied to the substrate is drawn into the first chamber of the through hole and held there by capillary force. The fluid is isolated from the second chamber in the hole due to the “bursting pressure” created at the interconnecting region between chambers, as shown in FIG. 14a. After removing excess first reagent, the second reagent can be applied to the top surface using a capillary bundle fluid delivery system, as shown in FIGS. 14b and 14c. Then a short pulse of driving pressure can be applied, which can either be negative pressure applied to the bottom side of the substrate to draw liquid in or positive pressure applied to the top side to push liquid in. In either case, the driving pressure is greater than the “bursting pressure” of the fluidic barrier between the first and second chamber. This results in the fluid in the first chamber bursting into the second chamber. Once the barrier is burst, capillary force takes over and draws liquid into the second chamber. Because the first and second chambers are connected, the second reagent on the top surface also is drawn into the through hole, as shown in FIG. 14d. Excess second reagent on the top surface can be removed and the container is ready for the loading of subsequent reagents (14e). This process can be repeated as many times as the number of chambers in the through hole (14f).

After all reagents are loaded into the through hole, mixing can be achieved by diffusion or alternatively, all reagents in different chambers of a through hole can be pumped into a larger chamber at the end of the through hole, where it will mix and incubate at a higher efficiency, as shown in FIG. 14f. The reaction results can be read from the through hole by optical or other means.

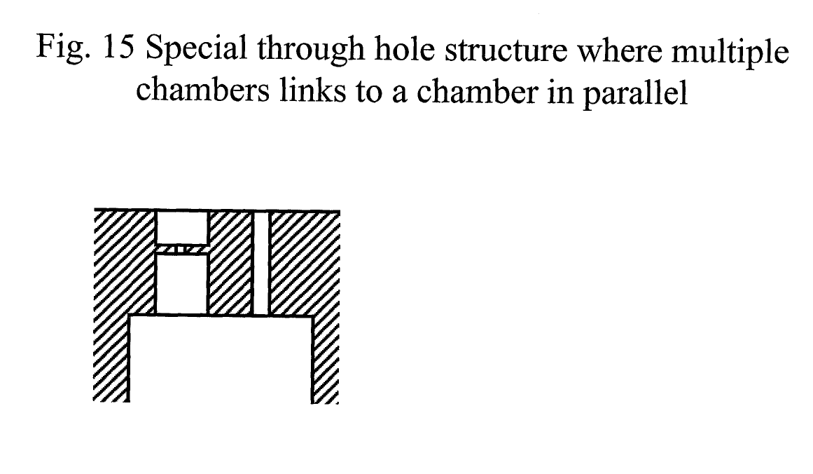

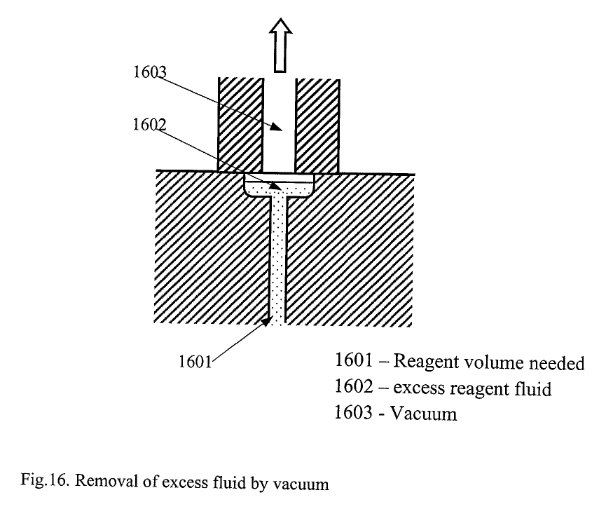

The structure and loading method described above is sequential for each container. FIG. 15 illustrates a different container structure, which comprises multiple chambers linked to a large mixing chamber in parallel (only two parallel chambers are shown in the figure). The different reagents can be loaded in parallel to different chambers of a container using e.g. a capillary fluid delivery system as described previously. The total required number of such fluid loading chambers in a container in the vast majority of applications is not very large because many reagents can be premixed in bulk prior to delivery to the substrate.

Excess Fluid Removal

In many fluid metering methods described above, fluid is delivered to the destination container, which is metered by an intrinsic reagent reservoir or chamber and any excess fluid outside the reservoir is removed. This invention provides a number of methods to remove excess fluids. These methods can be used alone or in combination.

The first method is to use vacuum force. The vacuum pressure has applied is less than the “bursting pressure” of the reservoir entrance that holds the metered fluid. As illustrated in FIG. 16, the substrate has a physical well with a much larger cross-section than the entrance of the metered fluid reservoir has. The capillary force in the reservoir is much greater than that in the well. A vacuum force selected to be smaller than the capillary force in the reservoir but larger than that in the well can be applied to remove excess fluid from the well while leaving the metered fluid in the reservoir intact.

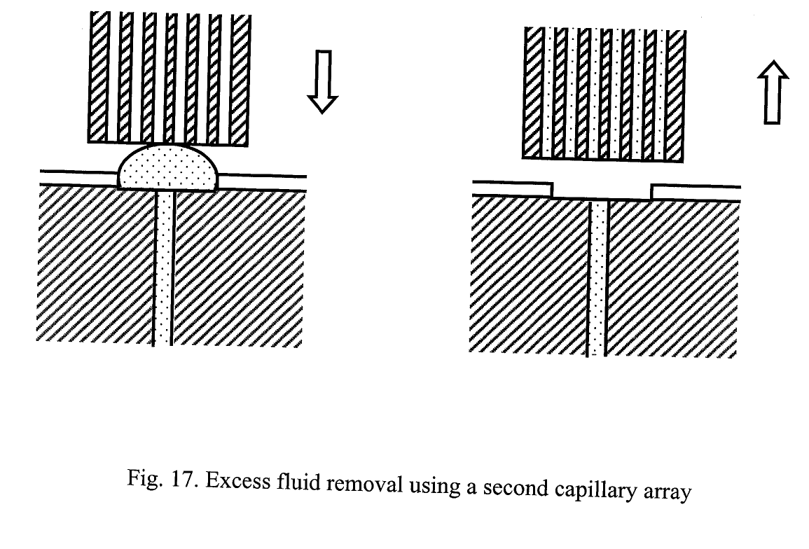

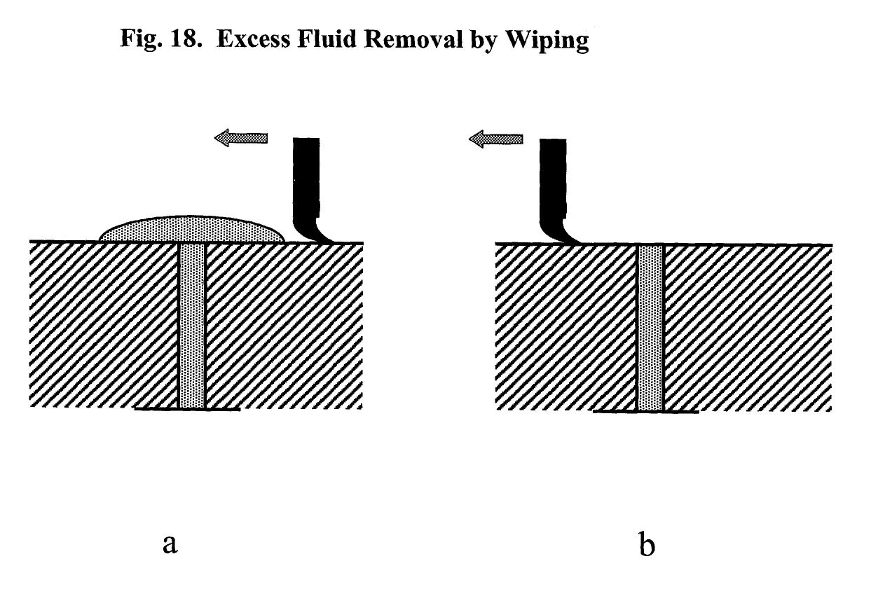

The second method is to blot the excess fluid with a suitable porous material, which can be a tissue or another capillary array for example. A tissue with suitable porous fiber composition can soak out the excess fluid positioned outside the metered reagent reservoir without removing liquid inside the reservoir. As illustrated in FIG. 17, a capillary array second with or without a matching through hole pattern whose capillaries have a capillary force slightly below that in the reservoir can be brought into contact with the excess fluid, which will draw the excess fluid outside the reservoir into its capillaries without removing fluid inside it.

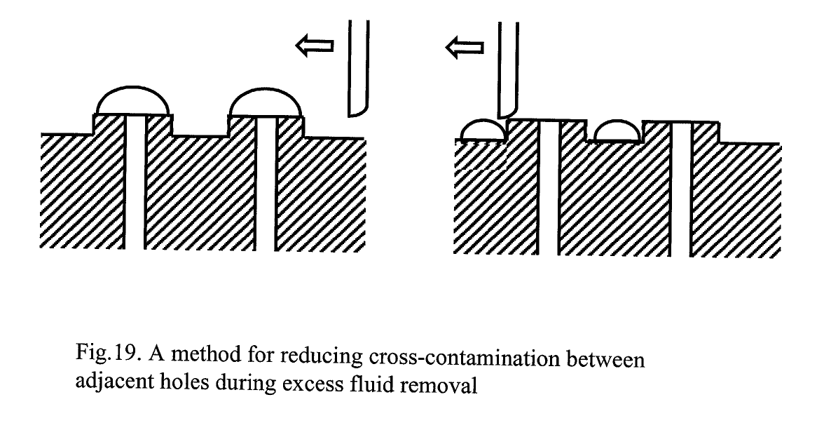

The third method is to mechanically wipe away excess fluid using a precision edge, as illustrated in FIG. 18. This method is suitable for structures where the excess fluid resides on a flat surface. The edge can be made of soft and non-porous material such as rubber or soft and porous material like a sponge. In this case, wiping and blotting is combined to remove the excess fluid. The edge can instead be an “air knife” that blows away excess fluid. This method may potentially introduce fluid cross-talk between different through holes if the pressure used is too high. This is not an issue if all fluid at the entrances to different fluid reservoirs are the same fluid. This invention also provides means to reduce fluid cross contamination. As illustrated in FIG. 19, each reservoir entrance is isolated geometrically by fabricating an island around it. Excess fluid falls into the gaps between these islands in a wiping action, thus reducing the chance of cross-talk between different reservoirs.

Islands may be formed by molding them into the surface during fabrication of the compound library substrate, for instance. Alternatively, islands may be formed by placing a patterned photoresist on areas that are to become islands and etching surface that is not protected by the photoresist. Likewise, the capillaries may be bound by an adhesive that has a substantially different etch rate from the capillaries, and the adhesive may be etched to remove a small amount, leaving capillaries standing slightly proud of the surface. This latter method obviates the need for masking the surface. Etchants include H₂SO₄ nanostripe, etc.

Optical Signal Readout

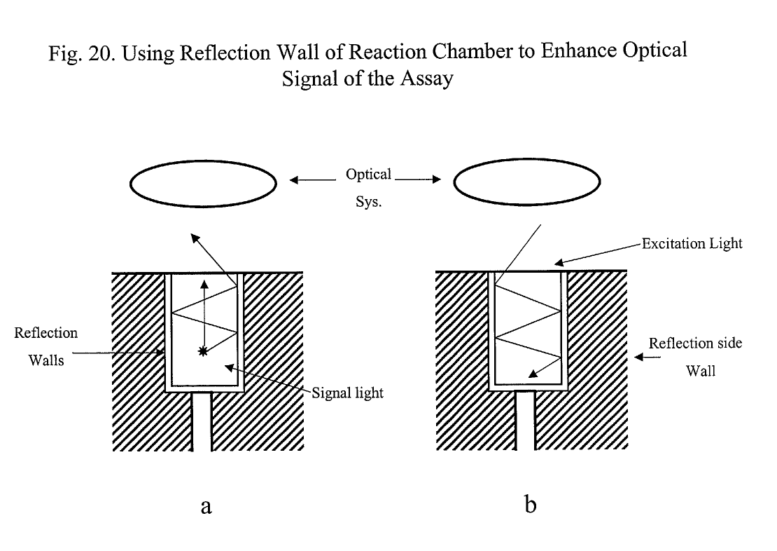

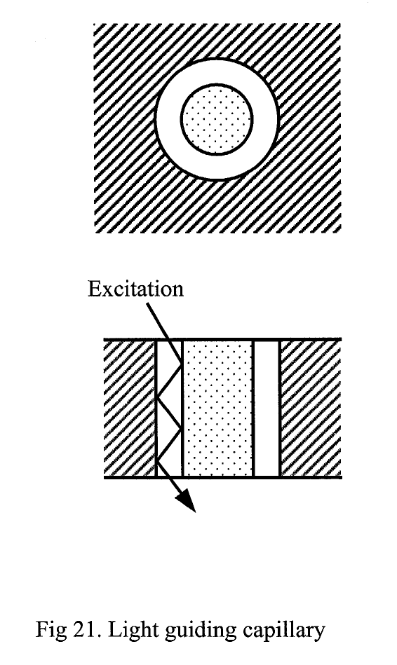

In a preferred embodiment, the invention provides features in individual through holes of the capillary array to assist readout of optical signals generated during the assay. FIG. 20 illustrates one embodiment of the design, where the inner wall of the mixing/reaction chamber of the capillary array is made highly reflective. This metal coating has two benefits: first, in a miniaturized structure, the wall between different reaction chambers may become too thin to efficiently block light from adjacent walls of wells. This may cause signal cross-talk and may reduce signal to noise ratio of the detection. A highly reflective layer is very efficient in attenuating light transmission between adjacent mixing/reaction chambers or through holes. Second, the metal coating enables a large percentage of the signal light that hits the wall that would otherwise be lost from an uncoated chamber to be eventually collected by the detection optics by directing the light to the optics through multiple reflections between chamber walls, as illustrated in FIG. 20a. Third, in fluorescence assays, one way to enhance signal to noise ratio of the detection is to enhance the fluorescence emission while suppressing excitation light that may be collected by the detection optics. A reaction chamber designed for fluorescence assays is built with a highly reflective side-wall and a bottom with a high degree of absorption. A major part of the excitation light will also bounce many times between the walls of the chamber, which excites the fluorescent marker multiple times thus multiplying the strength of the fluorescence signal (20b). Once the excitation light hits the bottom of the reaction chamber, it will be largely absorbed and thus will not bounce back to the opening, thus avoiding its collection by the detection system. The reflective layer in the chamber can be fabricated by coating a metal layer, such as gold, aluminum or copper by vapor deposition or sputtering. The coating is preferably only as thick as is needed to coat the walls to provide a reflective surface. Alternatively, the entire structure of the chamber can be built with metal material using e.g. an electric plating technique commonly employed in microfabrication of MEMS devices. In this technique, a substrate surface is first coated with a conductive layer, such as gold, suing vapor deposition. Then, a layer of photoresist is added. A lithography process and etching are employed to open up locations where metal structure is needed. Metal, such as nickel or copper is deposited in these designated locations by an electro plating process. On the other hand, a “grass” like surface feature can be fabricated on the bottom of the reaction chamber to significantly increase the absorption. Such surface features can be achieved through high ion strength bombardment during dry etching.

FIG. 21 illustrates another embodiment of the reaction chamber design that facilitates optical detection. In this design, a circular optical wave guide is built around the reaction chamber. The wave guide is formed by constructing a layer of optically transparent material with a higher refractive index than the adjacent regions. This layer can be made of pure silica, doped silica or suitable optical polymer. Such light guiding structure can be fabracated in a number of ways. In one embodiment, the light guiding layer is fabriacated on the inner wall of a silica tube preform by either doping Ge in the inner wall in a process termed MCVD (modified chemical vapor deposition) or doping fluorine on the outer wall using OCVD (outside chemical vapor deposition). The preform can be extruded into thin capillaries. A large number of such capillaries can be bonded together and cut to desired length to form a capillary array chip. Finally, this chip can be used as the capillary array compound library or may be bonded to a wafer containing other assay features to form the library. In another embodiment, the capillary array chip is prefabricated in silica or quartz. Ge or fluorine doping can be introduced to appropriate surface areas through ion assisted implantation. In fluorescent-based binding assays, the probe can be immobilized on the inner wall of the reaction chamber. The excitation light that enters the wave guide will generate an evanescent energy field along the inner wall of the reaction chamber. If the fluorescence labeled sample molecules bind the probe on the wall, they will be excited by the evanescent field and the signal light can be collected at either end of the wave guide. This configuration enables some very useful assays as described in a later section.

Additional Function Integrated to the Capillary Array Compound Library—Agent Immobilization

Immobilization of reagents and molecules on the substrate is used in preparing a variety of array embodiments of this invention. Improved methods for surface attachment chemistries are disclosed:

Protected-Aldehyde Silanization Agent Embodiments

In conventional methods, a surface functionalized aldehyde slide having surface immobilized functional groups with terminal aldehyde groups for attachment of polynucleotides or other biomolecules is typically prepared in a two-step method consisting of immobilization of an aminoalkyl silane on a substrate to provide terminal amino groups, followed by conversion of the terminal amino groups with glutaraldehyde to terminal aldehyde groups. However, such conventional methods may result in numerous undesired defects and side products, including residual amino groups and unreactive condensation products.

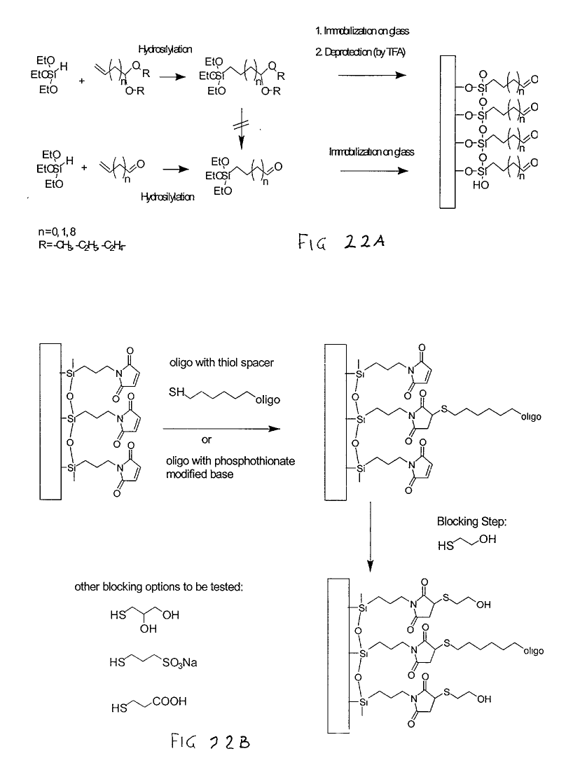

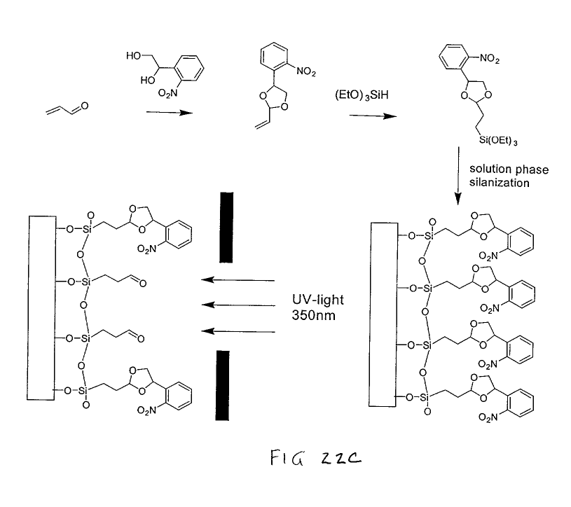

In one embodiment of this invention shown in FIG. 22A, a protected aldehyde silane is prepared and used to functionalize a substrate in a one step silanization reaction. Substrates functionalized in this reaction have no residual amino groups, and substantially lack non-aldehyde by-products. An acetal compound comprising a protected aldehyde is prepared by hydrosilylation reaction of triethoxysilane with an alkenyl acetal. A variety of carbon numbers for the alkenyl group may be utilized, providing a variety of alkyl chains for use as a spacer between the silane group and the acetal group, including isomeric mixtures of alkyl chains. The spacer group may also be a polymer or chemical group. The protected aldehyde product may be immobilized on substrates such as glass slides in a one step silanization reaction. The resulting substrate is functionalized with protected aldehyde groups that may be deprotected to provide a surface functionalized by aldehyde groups. Alternatively, a non-protected aldehyde silane may be prepared by hydrosilylation reaction of triethoxysilane with an alkenyl aldehyde. The silane aldehyde may be utilized in combination with the protected aldehyde product to functionalize a substrate.

A substrate may be functionalized with the protected aldehyde silane by a variety of techniques. For example, solution phase reaction of the protected aldehyde silane with the substrate surface may be used. Alternatively, vapor phase deposition of the protected aldehyde silane on the substrate surface may be used. In another embodiment, the substrate is cured after reaction of the protected aldehyde silane with the substrate. Curing may be performed over a wide range of temperatures for a period as long as one day, or longer. These conditions and techniques are well-known to those in the field.

The protected aldehyde silane of the functionalized substrate may be deprotected by a variety of reactions to produce active aldehyde groups. Deprotection may be performed with, for example, trifluoroacetic acid or hydrochloric acid, among others, resulting in a reactive surface aldehyde slide. Such slides are useful for attachment of polynucleotides and other biomolecules, for example, having amino linking groups.

Maleimide Silanization Agent Embodiments

Another composition and method for immobilization of reagents and molecules on the substrate are functional linker groups. In conventional methods, a surface functionalized slide is first prepared having attached functional linker groups with known ability to link, for example, polynucleotides or other biomolecules having various reactive groups such as amino groups, sulfhydryl groups, or phosphothionate groups. For example, an aminoalkyl silane is immobilized on a substrate to provide a surface having attached functional groups with terminal amino groups. In a second step, the functionalized substrate is reacted with a maleimide carboxylate to provide a reactive maleimide group attached to the surface linker group. The reactive maleimide groups are used to attach a polynucleotide. However, this conventional method typically results in undesirable residual amino groups.

In one embodiment of this invention shown in FIG. 22B, a maleimide silane is used to functionalize a substrate in a one step silanization reaction. In a maleimide silane, the reactive maleimide group is separated from the silane group by a spacer group which may have, for example, any one of a variety of carbon numbers to provide a variety of lengths of spacer chains between the two reactive groups. Substrates functionalized in this reaction have reactive maleimide groups immobilized on the surface, and no residual amino groups. The reactive maleimide groups on the surface may be reacted, for example, with sulfhydryl functionalized polynucleotides or other biomolecules to be attached to the surface. Unreacted maleimide groups may be blocked with various sulfhydryl-containing reagents, to provide a substrate with attached polynucleotides or other molecules, useful as probes. In further embodiments, the spacer may be one of a variety of polymers or chemical chains, for example, a polyethylene glycol. Various reagents may be added to the sulfhydryl functionalized reactant to prevent cross linking or other coupling of the molecules, such as a reagent to prevent disulfide bond formation.

A substrate may be functionalized in a one step silanization reaction with the maleimide silane by a variety of techniques. For example, solution phase reaction of the maleimide silane with the substrate surface may be used. Alternatively, vapor phase deposition of the maleimide silane on the substrate surface may be used. In further embodiments, the substrate may be cured after reaction of the maleimide silane with the surface. Curing may be performed over a wide range of temperatures for a period as long as one day, or longer.

Light Activation of Arrays

In further embodiments, the substrate may be chemically functionalized with surface-immobilized protected functional groups, where the protected functional groups are capable of being activated by absorption of light to provide reactive activated functional groups. The activated functional groups may be used to attach molecules, cells, or biomolecules to the surface. A mask or fiber optic bundle may be used to create a substrate having interspersed regions of activated and non-activated functional groups by irradiation of the substrate with light through the mask or fiber optic capillary bundle. The size, features, and morphology of the regions having activated functional groups are precisely controlled by the mask or fiber optic bundle. Biomolecules may be delivered to the surface and react to bind to the activated functional groups. Thus, the surface can be patterned to provide regions with bound biomolecules of precisely controlled size and morphology, regardless of the size or features of the region where the biomolecules were initially delivered to the surface.

In one embodiment, shown in FIG. 22C an aldehyde silane as discussed previously is used to functionalize the substrate by a silanization reaction. The aldehyde silane includes a photoreactive or photolabile group which, upon irradiation of the substrate, is cleaved from the surface immobilized silane, leaving a reactive aldehyde group attached to the substrate. The photolytic reaction can also be controlled by introducing a solvent to the substrate surface, or for example, by introducing one or more of various photosensitizer or photoinhibitor agents to the surface.

Other methods for binding biomolecules, such as polypeptides and proteins, nucleic acids, carbohydrates, lipids, and metabolic products or other ligands, as well as larger biological assemblies such as viruses, subcellular organelles, or even cells, to solid supports are well-known and characterized in the art. Generally, a biomolecule or other structure may be immobilized either covalently or non-covalently to the support; either type of binding may require modification of the biomolecule, or the support, or both. In some cases a binding pair, such as avidin/streptavidin and biotin, is used and one member of the pair is linked to the solid support while the other is linked to the biomolecule.

For nucleic acids, there are many techniques available and in common use, including covalent immobilization with or without pretreatment of support and/or nucleic acid (see, e.g., U.S. Pat. Nos. 6,048,695; 5,641,630; 5,554,744; 5,514,785; 5,215,882; 5,024,933; 4,937,188; 4,818,681; 4,806,631; Running. J. A. et .al., BioTechniques 8:276-277 (1990); Newton, C. R. et al. Nucl. Acids Res. 21:1155-1162 (1993)), non-covalent immobilization (e.g., U.S. Pat. No. 5,610,287), immobilization via avidin/streptavidinbiotin (e.g., Holnstrom, K. et al., Anal. Biochem. 209:278-283 (1993)). One very common substance used to prepare a glass surface to receive a nucleic acid sample is poly-L-lysine. See, e.g., DeRisi, et al. Nature Genetics 14: 457 (1996); Shalon et al. Genome Res. 6: 639 (1996); and Schena, et al., Science 270: 467 (1995). Other types of pre-derivatized glass supports are commercially available (e.g., silylated microscope slides). See, e.g., Schena, et al., Proc. Natl. Acad. Sci. (USA) 93: 10614 (1996).

For proteins, general techniques may be found in Methods in Enzymology, Vol. 44 (Immobilized Enzymes Edited by Klaus Mosbach, 1977); Vol. 135 (Immobilized Enzymes and Cells, Part B, Edited by Klaus Mosbach, 1987); Vol. 102 (Hormone Action, Part G: Calmodulin and Calcium-Binding Proteins, Edited by Anthony R. Means and Bert W. O'Malley, 1983); Academic Press, New York. Methods of covalent binding of proteins to supports may be found in, e.g., U.S. Pat. No. 5,602,207 and Zhang and Tam, Thazolidine formation as a general and site-specific conjugation method for synthetic peptides and proteins, Anal. Biochem. 233: 87-93 (1996), Support and method for immobilizing polypeptides.

Methods developed for the binding of antibodies to glass supports are of use, not only to bind antibodies, but other proteins as well. See, e.g., U.S. Pat. No. 5,646,001; Bhatia et al., Use of thiol-terminal silanes and heterobifunctional crosslinkers for immobilization of antibodies on silica surfaces, Anal. Biochem 178:408-413 (1989); Yanofsky et al., High affinity type I interleukin 1 receptor antagonists discovered by screening recombinant peptide libraries, PNAS USA 93: 7381-7386 (1996); Narang et al., A displacement flow immunosensor for explosive detection using microcapillaries, Anal. Chem. 69:2779-2785 (1997); Shriver-Lake et al., Biosens. Bioelect. 12:1101-1106 (1997).

Carbohydrates may also be immobilized to a solid support, either to bind substances to the carbohydrate, or to immobilize another moiety (e.g., a protein) which is attached to the carbohydrate. See, e.g., U.S. Pat. No. 6,231,733, Immobilized carbohydrate biosensor, Nilsson et al., 2001. The immobilized carbohydrate moiety may itself be specific for another type of biomolecule or structure, such as a protein, virus or a cell. A review of useful binding carbohydrate sequences can be found in e.g. Chemistry and Physics of Lipids, vol. 42, p. 153-172, 1986, and in Ann. Rev. Biochem., vol. 58, p. 309-350.

Methods for binding other biomolecules, as well as artificial molecules, substrates, ligands, and other molecules useful for binding biomolecules or biological substances of interest, depend on the nature of the substance to be bound and will be readily apparent to one of skill in the art. See, U.S. Pat. Nos. 5,817,470; 5,723,344; e.g., Weng et al., Proteomics 2:48-57 (2002); Zhou et al., Trends Biotechnol 10 (Suppl):S34-9 (2001); Mousses, et al., Curr Opin Chem Biol 6:97-101 (2002); Mirzabekov and Kolchinsky, Curr Opin Chem Biol 6:70-5 (2002); Reininger-Mack, Trends Biotechnol 20:56-61 (2002).

Surface Tension Pattern Fabrication

A “virtual well” may be formed by providing a hydrophilic patch surrounded by hydrophobic regions. An array of such patches can be fabricated using micro-fabrication techniques. A method for fabricating such an array by producing a substrate that contains hydrophilic and hydrophobic regions such that the localized surface energy differential concentrates and localizes reagents to specific regions adjacent to capillary ends on a substrate is described. Such concentration of reagents increases sensitivity of the assay by enhancing spot morphology and decreasing crosstalk potential.

In one embodiment substrates comprising capillary arrays or libraries may be tooled incorporating Micro Electro Mechanical Systems (“MEMS”) technology. Such substrates may be fabricated out of silicon and incorporate both the structural aspects of a microarray and the electronics for detection of reactions that occur on the microarray.

Masking Process for Marking Regions

One embodiment of the method utilizes masking technology to prepare localized areas on the surface for selective hydrophilization.

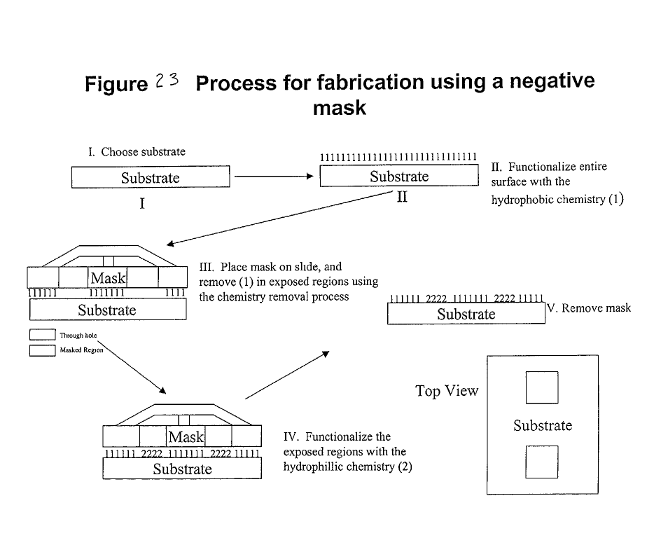

FIG. 23 shows a process for fabrication using a negative mask. In this method, the entire surface of a substrate is first functionalized with a hydrophobic (“1”) chemistry. Next a mask is placed on the substrate surface and the hydrophobic chemistry is removed from the exposed regions using e.g. a chemical removal process. The exposed (and stripped) regions are then functionalized with a hydrophilic chemistry (“2”).

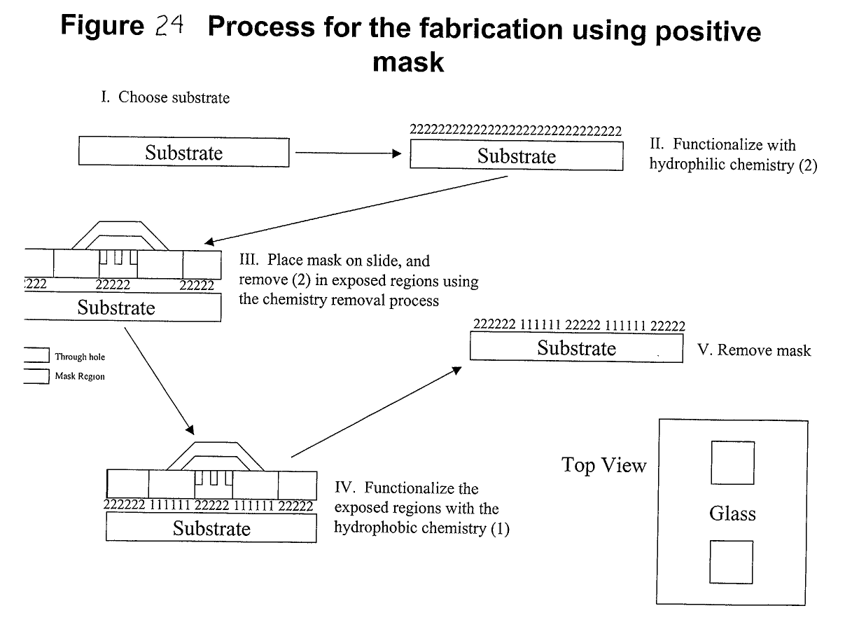

A positive masking process is illustrated in FIG. 24. The substrate surface is functionalized with a hydrophilic chemistry (“2”). Next, a mask is placed on the slide, and the hydrophilic chemistry (2) is removed from the exposed regions using e.g. removal chemistry. Then the exposed regions are functionalized with the hydrophobic chemistry (1) and the mask is removed.

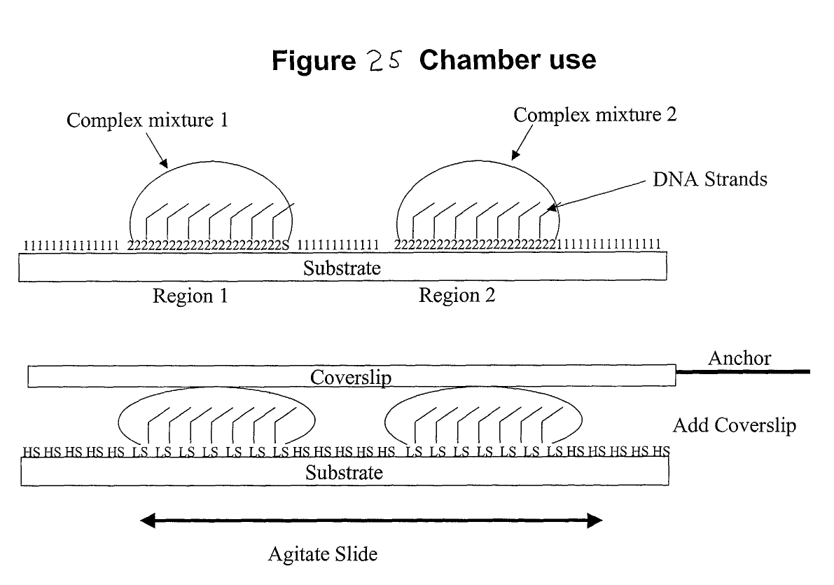

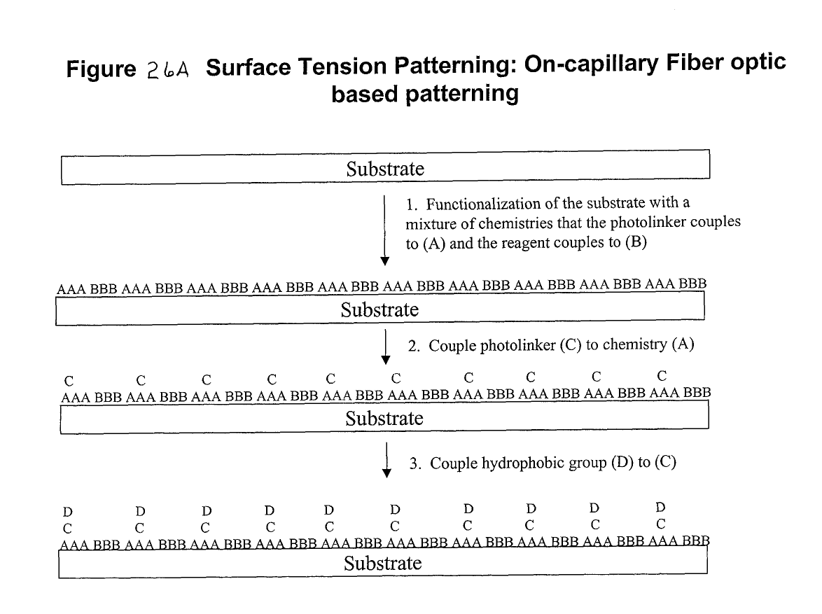

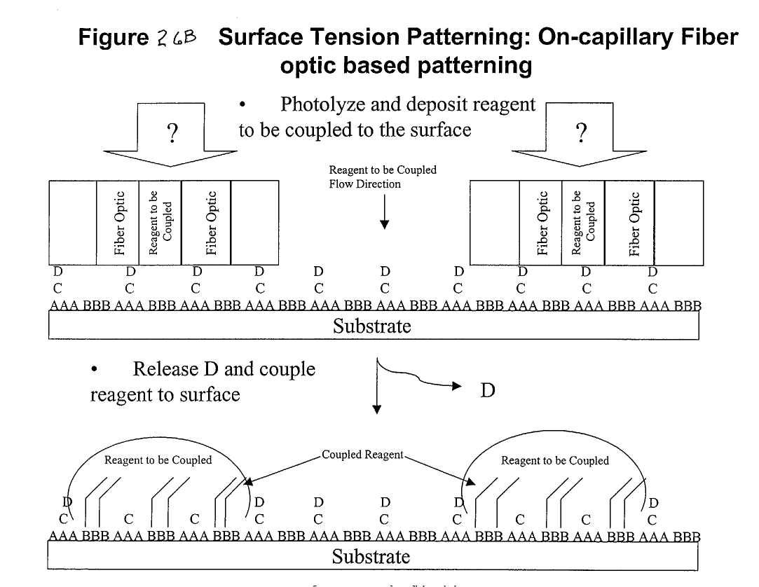

A typical use of such a chambered slide is illustrated in FIG. 25. Probe polynucleotide strands are immobilized on the hydrophilic regions of the substrate surface. Fluid droplets comprising potential targets for the probe nucleic acids are localized surrounding the probe regions by surface tension. An anchored coverslip is added to control dispersion of the target fluid. The target droplets adjacent the immobilized probe may then be mixed by agitating the substrate slide. Alternately the coverslip itself may be rotated, optionally by electromagnetic means, to agitate the solution and ensure mixing. The surface tension characteristic of the slide prevents the droplet from dispersing even in light of the relative movements of the slide and the coverslip. The movements of the slide and the coverslip are adjusted to generate less force than the surface tension holding the target fluid on the slide.

In one embodiment magnetic beads coupled to material that ensures the beads stay in the oil phase in a oil-water interface, are introduced with oil under the coverslip to ensure agitation and reduce evaporation.

Substrate Surface Patterning

In one embodiment, the invention relates to structures and methods for localizing and concentrating reagents at specific locations on substrates by surface tension patterning. The surface of the substrate may be patterned with regions of relatively higher or lower hydrophobicity interspersed with regions of relatively higher or lower hydrophilicity. The patterning of regions allows reagents to be localized according to their compatibility with the hydrophobicity or hydrophilicity of a region. In addition, surface tension effects are used to localize a reagent within an individual region. For example, in a hydrophobic region surrounded by regions of hydrophilicity, a relatively hydrophobic reagent may be localized and concentrated by differences in surface tension between the hydrophobic and surrounding hydrophilic regions. Conversely, a hydrophilic region may be used to localize and contain relatively hydrophilic reagents by differential surface tension from surrounding hydrophobic regions. The interspersed regions of hydrophobic and hydrophilic character may be used to form array structures on the substrate.

In one embodiment illustrated in FIGS. 26A and 26B, a pattern of interspersed regions of differing surface tension may be formed on a chemically functionalized surface by photolyzing regions of the surface to cleave a functional group from the surface, and contacting the photolyzed regions with a reagent that alters the surface tension of the photolyzed region. The photolysis may be carried out by any one of a variety of techniques, including laser scanning and fiber optic or other light waveguide transmission. In one embodiment, a fiber optic bundle is used to photolyze a pattern of regions on the substrate using light from a source. After photolysis of regions on the substrate, a reagent is added to the photolyzed regions where it becomes localized. Alternatively, the addition of reagent may occur at any time before, during, or after photolysis. In some embodiments, the fiber optic bundle includes hollow fiber optic fibers which may be used to deliver reagent to contact the substrate, as well as to provide light for photolysis.

In these embodiments, the substrate may initially be chemically functionalized with a variety of surface-immobilized functional groups (A and B) to provide interspersed regions having different attached functional groups, at least one of which is capable of receiving a photolinker group. This may be achieved, among other means, by masking technologies described in the previous section. Photolinker groups (C) are introduced to the surface and bind only to regions capable of binding the photolinker groups (in this case, A), providing a surface of interspersed regions, with and without attached photolinker groups. A hydrophobic or hydrophilic group (D) may be bound to the surface-immobilized photolinker groups to provide a surface of substantially uniform surface energy. A pattern of interspersed regions of differing surface tension may then be formed by selectively photolyzing regions of the surface to activate photolinker groups and release hydrophobic or hydrophilic groups that were attached to the photolinker groups. The photolysis reaction thereby photodeprotects some of the surface groups. The region of photolysis may be larger or smaller than the interspersed regions of differing surface tension. As described above, photolysis may be carried out by a number of methods, for example, using a bundle of hollow fiber optic strands. The difference in surface tension between the photolyzed and unphotolyzed regions may be increased by addition of a reagent containing coupling molecules to the photolyzed regions, which is localized to the photolyzed region, and allows the coupling molecules to couple to the surface, thereby increasing the difference in surface tension. The reagent containing coupling molecules may be coupled to the surface using, for example, the bundle of hollow fiber optic fibers.

Surface tension patterning may be carried out in further embodiments by using alternative methods of releasing hydrophobic or hydrophilic groups from the surface to create regions of differing surface energy. For example, additional approaches to photolytically release hydrophobic or hydrophilic groups from the surface are to utilize the methods of photolithography or ablation using ultraviolet light, which use a mask to select regions to be photolyzed. Using photolithographic methods, the substrate is coated with a photoresist, and the pattern is produced using light and a mask where regions of photoresist remain to selectively block reagents. Upon derivatizing the regions lacking photoresist with hydrophobic or hydrophilic groups, the photoresist may be removed to produce a pattern of interspersed regions having differing surface tension. The regions where photoresist was removed may be further derivatized by functional groups to increase the difference in surface energy between adjacent regions. A surface patterned by these methods can be used to localize reagents within regions of a particular character.

Techniques of ultraviolet (UV) ablation may also be used in surface tension patterning embodiments. The substrate is functionalized or coated with ablatable material or molecules. UV radiation is used to selectively ablate the coating from regions of the substrate by using a mask, thereby patterning the substrate. Regions from which ablatable material was removed may be further functionalized to create a pattern of interspersed regions of differing surface tension.

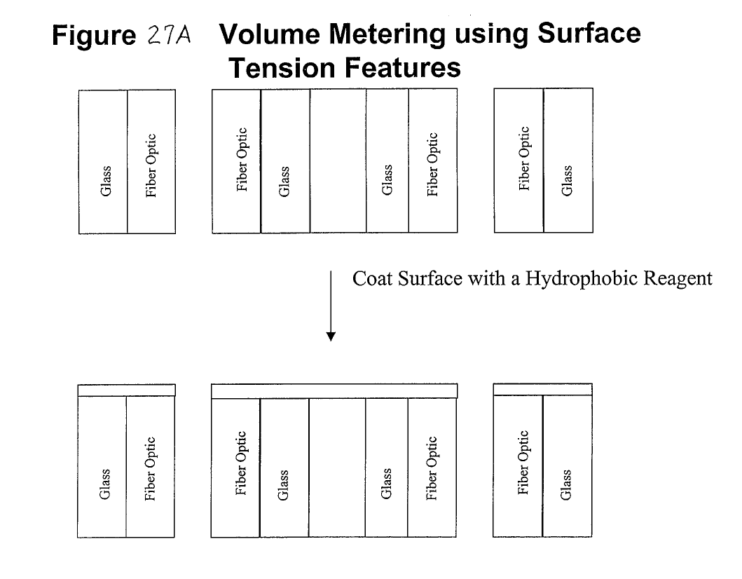

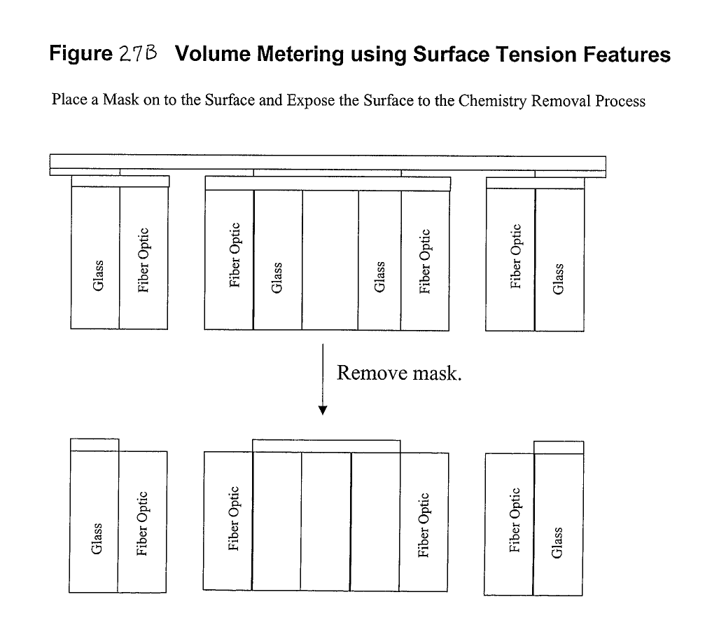

When the through holes in the capillary array have a light guiding ring around the hole, as illustrated in FIG. 21, tooling the surface for retaining metered volumes of reagents adjacent an inlet at the assay end of a capillary of an array is shown in FIGS. 27A and 27B. In the method, the entire surface of the substrate is coated with a hydrophobic agent, then a mask is applied to expose precise regions for application of removal chemistry reagents. When the exposed regions of the mask correspond with the capillary inlets, precisely tooled hydrophilic regions are created surrounding the assay or channel inlets.

Cartridge

The invention provides a cartridge that contains one or multiple above described capillary array compound libraries. The function of the cartridge is to protect the compounds from physical damage, chemical contamination and evaporation during shipment and pre-use storage. One or multiple DMSO reservoirs covered with semipermeable membrane are built in the cartridge. The cartridge is sealed in an airtight package (optionally filled with inert gas) for shipment and pre-use storage. These reservoirs generate a DMSO rich environment inside the package, as illustrated in FIG. 39.

Screening Station

This invention also provides a desktop-sized screening station that performs fully automated HTS operation in a personal setting, which includes the following basic functionalities:

The station loads the capillary array compound library in one or multiple cartridges if supplied on multiple chips, and the station stores the cartridge in a suitable, controlled environment chamber.

The station accepts and routes additional reagents needed for the HTS assay, which usually include an enzyme, a substrate and buffer, and the station may preprocess these reagents, which may involve dilution and pre-mixing.

In cases where the compound is dried in the through hole of the library before shipping to users, the screening station may redissolve the compound in pure DMSO. Preferably, the concentration of DMSO or other polar solvent is no more than

The station delivers reagents to the capillary array compound library, facilitates reagent metering by removing excess fluids by e.g. tilting the library, vacuuming, squeegying, and/or air-blowing the surface. The station may also initiate mixing of these reagents with compounds in separated mixing/reaction micro-chambers of e.g. a capillary array.

The station provides suitable environmental chambers for the capillary array to incubate.

The station has an integrated detection system to detect signals indicating the results of the assay.

The station has the capability to clean and regenerate various surfaces that have been used by previous screening assays and prepare for the next HTS operation.

Pre-Screen Environmental Chamber

The shipping package seal will be opened before the capillary array cartridge is inserted in the pre-screen environmental chamber. The chamber provides a clean, DMSO rich and cooled, preferably to 4° C., environment to ensure that the compound solutions stored in the through holes of the capillary array remains filly effective over a prolonged period of time before screening.

Reagent Cartridge

In one particular embodiment of the screening station design, a single-use reagent cartridge is provided, which has separate reservoirs for multiple reagents needed for the HTS assay. Reagents can be loaded into the cartridge outside the machine and then the cartridge may be inserted into a designated port on the screening station. Predilution or mixing of reagents, if needed for the assay can also be conducted on the cartridge. This can reduce the burden of cleaning after the HTS assay.

Re-Dissolution of Dried Compounds

This additional step is only needed if the compound is shipped dry in the library. Pure DMSO may be introduced to the capillary compound library, which is drawn into the compound storage chamber by capillary force. Excess DMSO is removed. After a certain incubation period, the compound powder will be re-dissolved into the DMSO solution and ready for HTS assay.

Assaying Station

The invented screening station may provide a mechanism to remove individual capillary arrays from a cartridge without the need for manual handling. The cartridge is loaded on an assaying station, which has fluid handling capabilities to enable the delivery of multiple reagents from their storage cartridge described above to the capillary array, removal of excess fluids after reagent metering and mixing them with compounds in different mixing chambers. This invention provides a number of different fluid handling mechanisms, which are related to the structure of the through holes in the capillary array or library.

Because the reagents used for HTS assays are common to every compound holding through holes in the capillary array, one method to deliver the reagent to the capillary array is to flood the reagent liquid onto the assay surface of the capillary array. The fluid metering devices built on the capillary array, such as the virtual or physical wells described previously, will hold a designated volume of fluid and the excess fluid will be removed by e.g. tilting the substrate to allow excess fluid to run off.

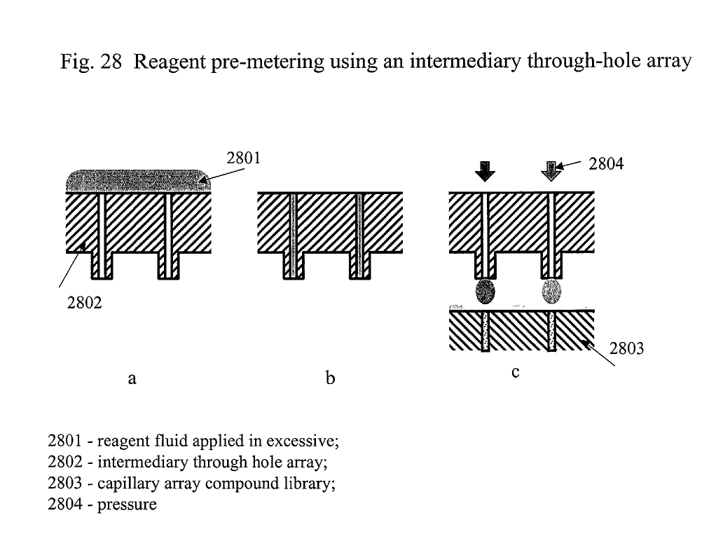

Another delivery method is a two-stage approach. As illustrated in FIG. 28, a chip having an array of through holes serves as an intermediary liquid delivery device. The through holes in the chip spatially match the compound holding sites in the capillary array compound library. The inner volume of each through hole is slightly larger than the reagent volume needed for mixing with each compound. The function of this through hole chip is to pre-meter and distribute the reagent to each compound contained in the chip or library. The bulk reagent is delivered to the top surface of the chip in a flooding fashion. The reagent solutions fill each through hole by capillary force (FIG. 28a). The excess reagent fluid is then removed from the top surface of the chip (FIG. 28b, as described previously). Further, the through holes in the chip are aligned with the compound holding through holes in the capillary array or library. The reagent is driven out of the intermediary chip onto the capillary array compound library by pressure (FIG. 28c). Because the reagent is pre-metered for each compound, the amount of excess fluid is greatly reduced, which reduces the chance of cross-contamination between compounds.

The following are a number of examples describing detailed steps of typical HTS assays carried out in a screening station. The enzymatic assay involves adding an enzyme and a substrate in two steps and mixing them with the compound contained in the capillary. Other assays can be conducted in a similar fashion. Most of these assay steps have been described in previous sections. the following explanation provides a more integrated presentation of the operation of the entire system.

Enzymatic Assay with Multiple Use Compound Library

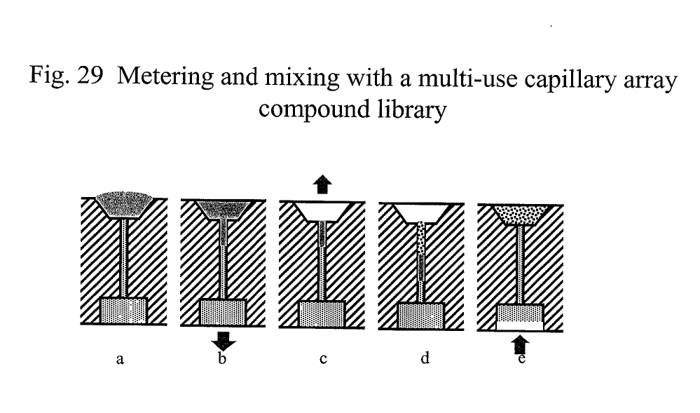

FIG. 29 illustrates the operational steps to carry out an enzymatic assay using a capillary array compound library designed for multiple uses. The through hole structure comprises a micro-reaction well linked to a large compound reservoir through a long and narrow path. First, the enzyme solution is deposited on the assay surface in bulk, filling the micro-reaction wells (a). Second, a negative pressure is applied to the reservoir side to draw a defined amount of enzyme into the narrow path region to dispense some of this compound(b). Third, the excess enzyme in the well is removed by vacuum aspiration from the top (c). The same operations from 1^{st} to 3^{rd} step are carried out for substrate solution in 4^{th} to 6^{th} steps. As a result, there are two short slugs of enzyme and substrate fluids in the narrow path as well as some assaying compound (d). Seventh, a positive pressure is applied to the reservoir side which pushes both fluids plus a defined amount of compound out into the micro-reaction well where they mix, incubate and are read by the detection system. After readout, the mixture in the micro well is removed and washed with buffer. The device is ready for the next screen. In this particular capillary array compound library, volume metering is achieved through precise pressure acting on the narrow path or channel, which functions as a fluid regulator.

Enzymatic Assay with Single Use Library and Virtual Well Metering

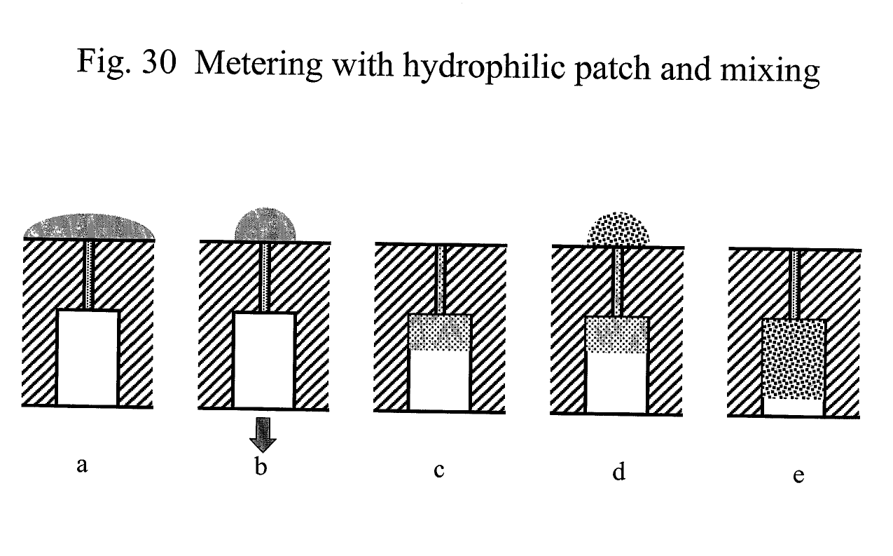

FIG. 30 illustrates the operational steps to carry out an enzymatic assay using a single use capillary array compound library. The through hole structure comprises a “virtual well” on the assay surface. The mixing/reaction chamber is linked to the virtual well through a capillary portion, which stores the compound. First, the enzyme solution is deposited on the assay surface in bulk (a). Second, the surface is tilted to remove the excess fluid. A defined volume droplet is retained by the hydrophilic patch around the through hole entrance (b). Third, a negative pressure is applied to the mixing chamber side to draw in the entire droplet through the compound chamber into the mixing chamber. The enzyme will start mixing with the substrate (c). Steps 4^{th} to 6^{th} repeat steps 1^{st} to 3^{rd} (omitting the analogous step to step (a) from the figures) but use the substrate solution in place of the enzyme solution. All three reagents mix in the mixing chamber (d).

Enzymatic Assay with Chamber Metering

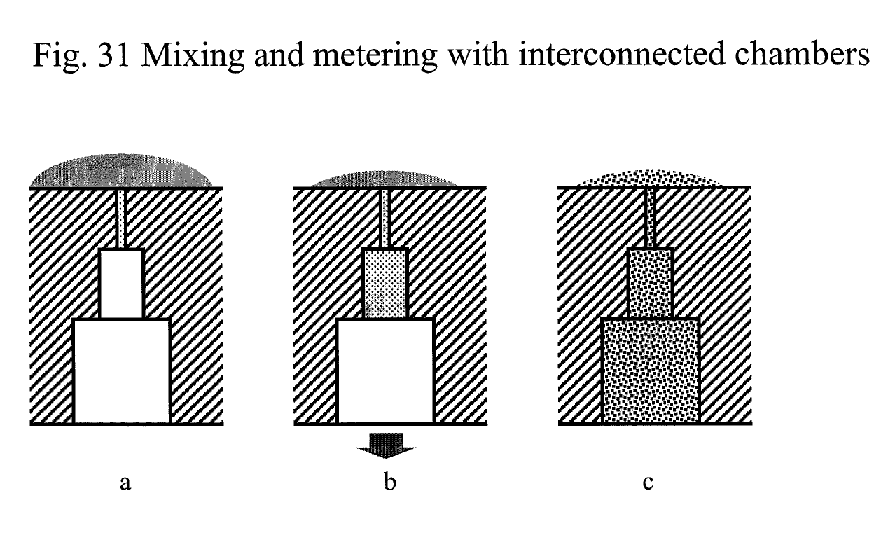

FIG. 31 illustrates the operational steps to carry out an enzymatic assay using a single use capillary array compound library. The through hole structure comprises three interconnected chambers. The thin capillary chamber closest to the assay surface is used to store the compound. First, the enzyme solution is delivered to the assay surface in bulk (a). Second, a short duration of negative pressure is applied to the mixing chamber side, which breaks the fluid barrier formed by the large and abrupt expansion between the compound chamber and its adjacent enzyme mixing chamber. The fluid fills the second chamber due to capillary force drawing in a define the volume of enzyme, which mixes with the compound in the enzyme mixing chamber (b). After removing excess enzyme from the assay surface, steps 3^{rd} and 4^{th} will be carried out to the substrate similar to steps 1^{st} and 2^{nd} The fluid barrier between the enzyme mixing chamber and final mixing chamber is overcome, and the compound, enzyme and substrate mix in the two chambers (c).

Enzymatic Assay Using Through Hole Metering

FIG. 40 illustrates the operational steps to carry out an enzymatic assay using a single use capillary array compound library chip and multiple separate reagent metering chips. The through hole in the library chip comprises two interconnected chambers. The thin capillary chamber closest to the assay surface is used to store the compound. The much larger chamber is used for reagent mixing and reaction. Separate enzyme and substrate metering chip are constructed which have a through hole array at the same pitch and spatial pattern as the through holes in the library chip. The inner space of each through hole in the enzyme or substrate metering chip is designed to be same as the volumes of enzyme and substrate solutions required for the assay, respectively. In most HTS applications, the volumes of enzyme and substrate 50 to 500 times larger than that of the compound. Therefore, it is desirable that the diameter and volume of the through holes in the enzyme or substrate metering plate is much larger than that of the compound storage chamber in the library chip. As illustrated in FIG. 39, the enzyme and substrate solutions are first delivered to through hole plate A and B, respectively and metered in a process described previously. Then the through hole in plate A is aligned with a compound storage chamber on the library chip and a fluid connection is established (a). Second, a negative pressure is applied to the mixing chamber side to draw not only all the compound but also all the enzyme in the through hole a separate chip into the mixing chamber (b). Then Plate B is aligned to the library chip (c) and a negative pressure at the mixing chamber side (or a positive pressure at the Plate B side) is used to draw (or push) all substrate into the mixing chamber (d), where the three solutions will mix and incubate.

Heterogeneous Protein or Cell Assays

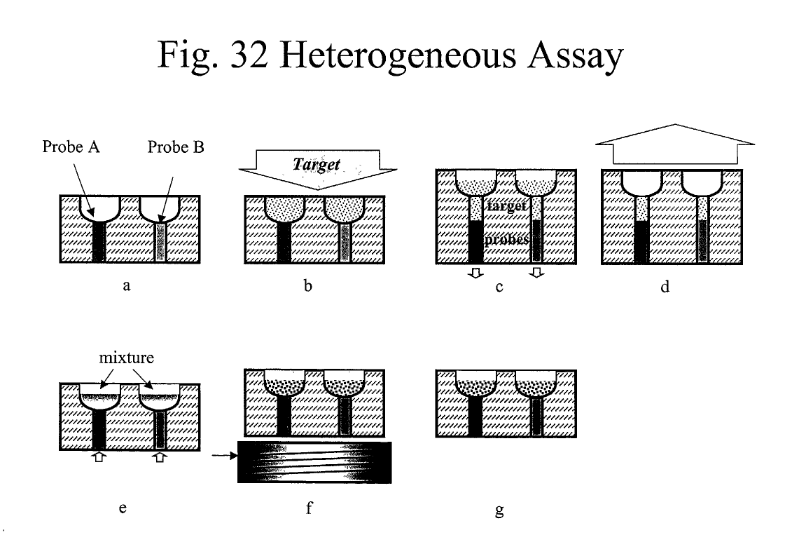

The invention can readily be applied in protein array fabrication, assaying and readout system. FIG. 32 illustrates an embodiment of an assay involving protein arrays or cell arrays. A library of antigens or antibodies is attached to magnetic beads 460 (Dynal Corporation) using standard biochemical protocols. The method discussed above is used to mix the sample and proteins or cells of the library. The reaction head may be sealed using e.g. a glass or polymeric plate 470 as illustrated at step (e), and the reaction head may be transported to a separate magnetic head 480, where the plate is removed, a washing fluid is placed into the chambers as part of the washing cycle, the beads are subjected to a magnetic field generated by the head (e.g. an electromagnet), and the fluid is removed by aspirating it but the beads are held in place by the magnetic field. Washing steps are necessary in heterogeneous assays, and washing is greatly facilitated by use of paramagnetic beads that are retained in the reaction chamber by the magnetic field generated by the electromagnet when the wash liquid is removed. The system is then demagnetized, and the reaction head is moved to a position for imaging e.g., using a fluorescence scanner. Once scanning is completed, the magnetic beads are aspirated from the reaction chambers, the reaction chambers are washed as described previously, and the reaction head is prepared for another cycle.

Incubation Chamber

After mixing, one or multiple capillary array compound libraries can be placed in an incubator, which maintains a high humidity and suitable temperature for a designated duration for reaction incubation.

Detection System

The screening station provided by the invention provides an integrated detection system to detect optical signals generated by the HTS assay. Detection of biomolecular reactions on the invented system may be carried out using colorimetric, fluorometric, electrochemical, and/or electronic detection labels. Optical detection modes may include absorption, colorimetric, chemical luminescence, fluorescence intensity, FRET, time-resolved fluorescence and fluorescence polarization. When the reaction occurs in the reaction well or the virtual well on the substrate surface (using surface tension to restrict fluid flow), the reaction may be followed using standard detection techniques such as those involving optical, CCD, CMOS or laser optics. Where the reaction occurs within the capillaries and the reaction product is not extruded from the through hole (or the reaction is followed in real time) a variety of methods may be used to extract the signal from within the capillary. Use of an optical fiber capillary coupled to a detection (CCD, C-MOS) device at a remote end will allow a technician to follow a reaction. Alternately, the walls of the capillary may be lined with light reflective material (as shown in FIG. 20) to amplify a light signal such as that generated by a fluorescent probe. In another embodiment, the substrate itself may be fabricated from a transparent material. Examples of some detection labels suitable for the present invention are discussed below:

Fluorescent Probes

Interaction of the target and chemical compounds can be assayed by detecting the fluorescence emission (intrinsic or extrinsic probes) of a target system labeled with fluorescent molecules such as, e.g., DAPI, Texas red and fluorescein. The detection system can be a charged coupled device (CCD) based fluorescence imaging system. In one illustrative but non-limiting example of CCD-based fluorescence imaging and analysis, fluorescence images of 5 mm×7 mm regions of the reaction heads or through hole plates are obtained using a lx magnification imaging system coupled to a 12 bit CCD camera (e.g., Photometries KAF 1400 chip). Excitation light, supplied from a mercury arc lamp equipped with a computer controlled filter wheel, is projected onto the reaction head using a quartz prism. After impacting the reaction head the light is reflected to the CCD detector. A multiband pass filter (e.g., P8100, Chroma Technology, Brattleboro Vt.) is used in the emission light path. Exposure times are less than one second for DAPI, and between 0.5 and 2 sec for fluorescein and Texas red. Images are analyzed with software that segments the array targets based on the DAPI image, subtracts local background, and calculates several characteristics of the signals for each target including the total intensity of each fluorochrome, the fluorescein/Texas red intensity ratio, and the slope of the scatter plot of the fluorescein and Texas red intensities for each pixel.

A microarray or compound library comprising a random bundle may have software associated with it that provides data which correlates the identity of the target or probe molecules with a particular location on the reaction head, as discussed above. The software may be provided as a database providing this correlation and may be on a portable medium such as a CDROM or may be downloaded to a user's equipment via a telephone line, cable modem, satellite link, or other form of data communication. The software may also be programmed into an EPROM located on the library. The software may be loaded into a computer or into dedicated equipment associated with a scanner, such that the hybridization pattern read by the scanner can be translated into information on the target molecules or probe molecules that have hybridized (or otherwise associated) on the substrate.

Fluorescence Quenching and Light-Up Probes

In the systems of the present invention, the analyte-probe moiety is detected. There are three basic methods of detection: first, no label, in which an intrinsic property of the probe-analyte structure which is different from that of probe or analyte alone is detected; second, a single label, either on probe or analyte, either produces a signal which may be measured after unbound label is removed, or an existing signal is altered in a measurable way upon formation of the probe-analyte structure, thus obviating the requirement of removal of unbound label; third, label pairs, in which at least one label on the probe and one label on the analyte interact upon binding to produce a signal, which also obviates the need for removal of unbound label. Any of these may be used in the methods of the invention.

Label one member of a pair: Several methods have been developed and are known to those of skill in the art for using a single label which is altered upon formation of the analyte-probe pair. For nucleic acids, the use of light-up probes in nucleic acid analysis allows one member of a probe-analyte pair to be labeled in such a way that binding of probe and analyte results in a large increase in fluorescence signal. The use of such probes is known in the art and discussed in, e.g., U.S. Pat. No. 6,329,144; Svanvik et al., Anal Biochem 281:26-35 (2000). Other methods include probes composed of an oligodeoxyribonucleotide equipped with a ruthenium complex, where hybridization can be demonstrated from measurements of the probe fluorescence lifetime (Bannwarth et al., Helvetica Chimica Acta, 71, 2085, 1988); a probe composed of a DNA-chain modified with a metal-ligand complex whose fluorescence intensity increases upon hybridization (U.S. Pat. No. 5,157,032); a probe composed of an oligonucleotide modified with pyrene, which under optimal conditions gives a 20-fold increase in fluorescence upon hybridization (Yamana et al., Nucl. & Nucl. 11 (2-4), 383, (1992); probes composed of an oligonucleotide and an asymmetric cyanine dye, whose fluorescence properties, such as fluorescence polarization, fluorescence lifetime and fluorescence intensity, are changed upon hybridization (EP 0710 668 A2, U.S. Pat. No. 5,597,696; Ishiguro et al., Nucl. Acids Res. 24, 4992, (1996). Methods in which two probes are used to analyze a single analyte also are applicable, such as a probe based on simultaneous hybridization of two DNA-based probes to close-lying sequences, where one probe is modified in the 3′-terminus of the DNA chain with a donor fluorophore and the other probe is modified in the 5′-terminus with an acceptor fluorophore. When they are in proximity fluorescence energy is transferred from the donor to the acceptor fluorophore, which can be detected. The fluorophores are far apart in solution, but are brought together when the probes hybridize to TS by binding with the 3′-terminus of one probe next to the 5′-terminus of the other probe. See, e.g. Heller et al., (EPA 070685) and Cardullo et al., (Proc. Natl. Acad. Sci. USA, 85, 8790-8794, 1988).

Interacting label pairs: In one mode of detection, the probe and the analyte each comprises a member of an interacting label pair. The members interact when in close proximity, such that association of the members on the two probes results in generation of a signal. By “signal” is meant a measurable characteristic. The signal may increase or decrease upon association of the members of the interacting label pair. For example, if the interacting label pair comprises a fluorophore and a quencher, association of the members of the pair generates a detectable signal due to a decrease in light energy emitted by the fluorophore in response to illumination. Or, for example, if the interacting label pair comprises subunits of an enzyme, association of the members of the pair generates a detectable signal which is an increase in the rate of the reaction catalyzed by the enzyme. Each member of the interacting pair may comprise one or more than one molecule or structure. The change in signal may be all-or-none (for example, if the moieties are an enzyme-inhibitor pair, where the enzyme is either active or inactive) or vary over a range (for example, if the moieties are a fluorophore-quencher pair). The change is characteristic for the moieties (labels) employed. In some embodiments, two or more kinds of interacting label pairs may be used in a single sample in order to differentiate, e.g., different target analyte acid sequences. The detectable signal may be, e.g., a characteristic light signal that results from stimulating at least one member of a fluorescence resonance energy transfer (FRET) pair. Another example of a detectable signal is a color change that results from the action of an enzyme/suppressor pair or an enzyme/cofactor pair on a substrate to form a detectable product. In some embodiments, the signal is a reduction or absence in detectable signal.

Various combinations of moieties (labels) which are capable of producing a detectable signal which differs depending on their degree of proximity, can be used. Any combination or number of moieties (labels) which interact so as to produce a measurable change upon change in the proximity of the moieties (labels) is sufficient; hence, more than one pair of moieties (labels) may be used. Nor is it required that there be a one-to-one correspondence between members of an interacting label pair, especially where one member can affect, or be affected by, more than one molecule of the other member.

Interacting label pairs useful in the present invention are known in the art, see, e.g., U.S. Pat. Nos. 5,688,648 (Mathies et. al); 5,340,716; 3,999,345; 4,174,384; and 4,261,968 (Ullman et al.); 4,996,143 and 5,565,322 (Heller et al.); 5,709,994 (Pease et al.); and 5,925,517 (Tyagi et al.). Examples of suitable moieties (labels) in which one member quenches another include a fluorescent label, a radioluminescent label, a chemiluminescent label, a bioluminescent label, an electrochemiluminescent label, and an enzyme-inhibitor combination. In some embodiments, the interacting moieties (labels) may generate little or no signal when in close proximity and generate a greater signal when separated. In other embodiments, the interacting moieties (labels) produce little or no signal when separated, and a greater signal when in close proximity. Examples of the latter such moieties (labels) are an enzyme and its cofactor and fragments or subunits of enzymes that must be close to each other for the enzyme to be active.

If fluorescent labels are used, labels are chosen such that fluorescence resonance energy transfer is the mode of interaction between the two labels. In such cases, the signal generated by the association of the labels could be an increase in the lifetime of the excited state of one label, a complete or partial quenching of the fluorescence of one label, an enhancement of the fluorescence of one label or a depolarization of the fluorescence of one label. The labels could be excited with a narrow wavelength band of radiation or a wide wavelength band of radiation. Similarly, the emitted radiation could be monitored in a narrow or a wide range of wavelengths. Examples of such pairs are fluorescein/sulforhodamine 101, fluorescein/pyrenebutanoate, fluorescein/fluorescein, acridine/fluorescein, acridine/sulforhodamine 101, fluorescein/ethenoadenosine, fluorescein/eosin, fluorescein/erythrosin and anthranilamide-3-nitrotyrosine/fluorescein. Other such label pairs will be apparent to those skilled in the art.

Various combinations of dye moieties (labels), which are capable of energy transfer when in close spatial proximity, can also be used. For example, interacting moieties (labels) may be a donor-acceptor dye pair, capable of energy transfer when in close spatial proximity. Label 1 may be a fluorescent dye and label 2 a quencher which is able to absorb the fluorescence signal of label 1 by an energy transfer mechanism. Alternatively, the moieties (labels) may be ligands for reporter molecules which can interact with each other when brought in close spatial proximity, the interaction of which prevents or enables activity of one of the reporter molecules. Examples for suitable combinations of reporter groups useful for the methods of the invention are enzyme-inhibitor combination, reporter molecules which when reacting with one another form an active enzyme molecule, and the like. The association of the two interacting reporter groups is detectable and indicative of the presence of one or more target analyte(s) in a sample, the quantity of analyte(s) in a sample or degree of identity of the analyte with a reference, e.g. the degree of identity of a sequence of nucleic acid analyte(s) to that of a reference nucleic acid sequence(s).

Either the probe or the analyte, or both, may optionally incorporate more than one moiety to make up its member of the interacting label pair. The moieties may be located anywhere on the probes or analyte as long as they are capable of interacting when probe and analyte bind together. The moieties may be attached to one end of the probe or analyte, or may be attached to the interior of the probe or analyte. Members of the interacting label pairs may be attached to probes either during or post-synthesis of the probes. The attachment of a member of an interacting label pair to the probe is preferably covalent, and means of attachment will vary depending on the probe and the member of the interacting label pair, such means being readily apparent to one of skill in the art. Similar considerations apply to attachment of a probe pair to analyte.

An example of a fluorescent-quencher pair is the fluorescent moiety 5->(2-aminoethyl)aminonaphthalene-1-sulfonic acid (EDANS) and quenching moiety 4-(4-dimethylaminophenylazo)benzoic acid (DABCYL). For EDANS and DABCYL, quenching is essentially eliminated by a separation of 60 Angstroms.

Applications of the Invention

High Throughput Screening for Receptor Binding in Fiber Optic Capillaries

A high throughput screen for a receptor binding assay in capillary arrays involves (a) strategies for immobilizing a probe molecule (antibody, receptor) within a capillary and (b) an approach for carrying out the assay to measure binding kinetics.

Antibody Immobilization Strategies

There are several strategies for immobilizing an antibody on the inner wall of a capillary:

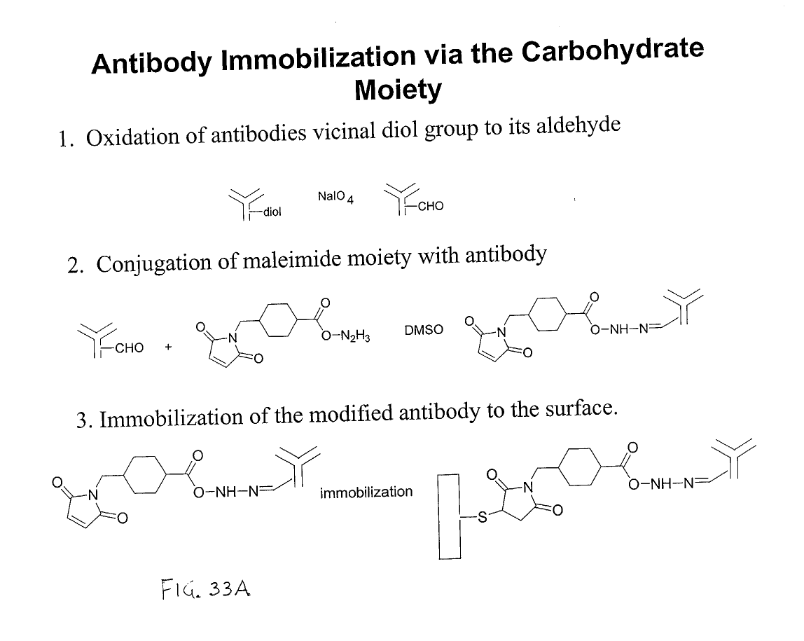

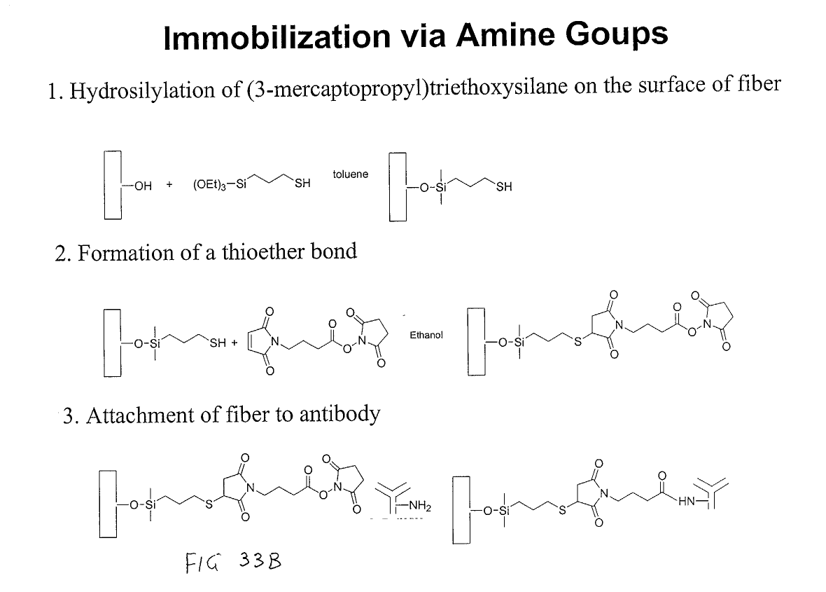

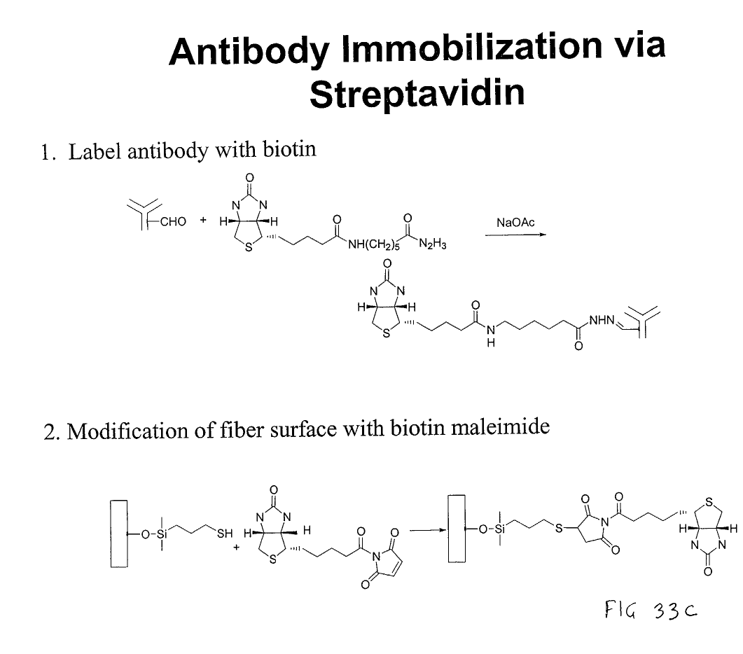

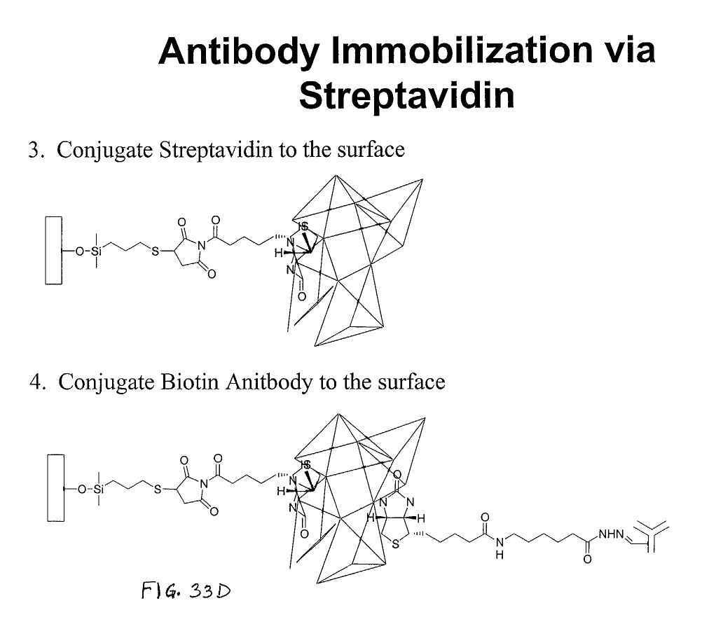

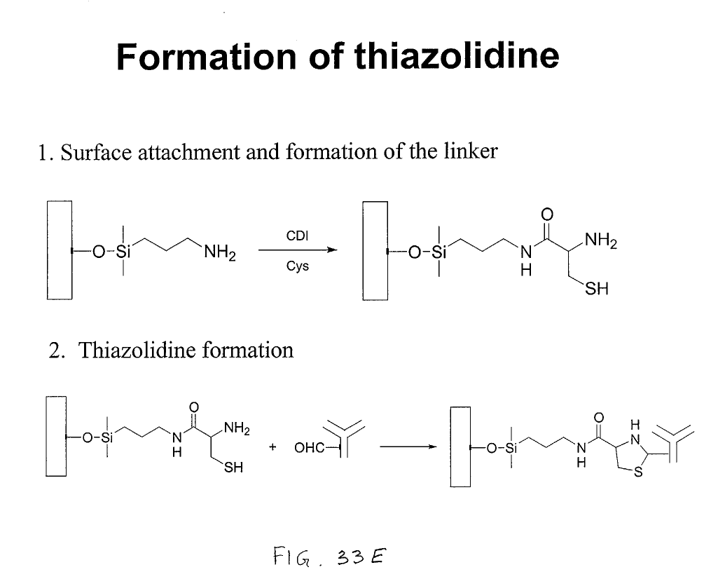

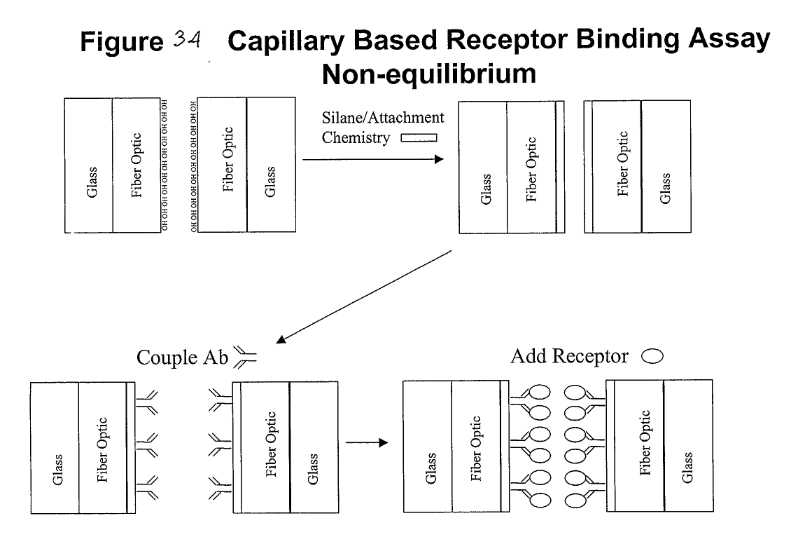

A first embodiment shown in FIG. 33A uses immobilization via the carbohydrate moiety. The process involves oxidation of the antibody's vicinal diol group to its aldehyde followed by conjugation of a maleimide moiety with the antibody and immobilization of the modified antibody to the surface.

FIG. 33B illustrates immobilization via amine groups by hydrosilylation of (3-mercaptopropyl) triethoxysilane on the surface of fiber followed by formation of a thioether bond and then attachment of fiber to antibody.

FIGS. 33C and 33D illustrates immobilization via avidin-biotin binding. The antibody is labeled with biotin. The fiber surface is modified with biotin maleimide. Then Streptavidin is conjugated to the surface followed by conjugation of biotinylated antibody to the surface.

FIG. 33E illustrates immobilization via surface attachment, linker formation and thiazolidine formation.

Receptor Binding Assay under Non-Equilibrium Conditions

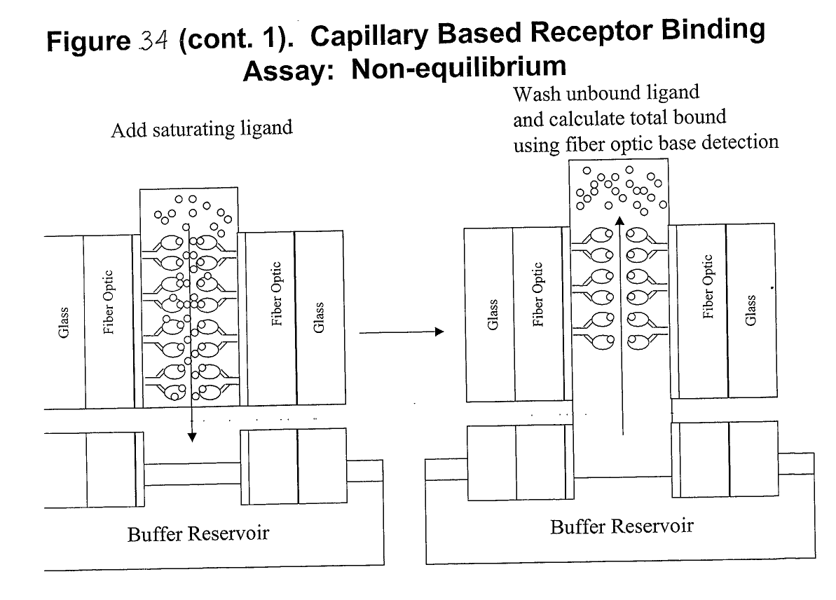

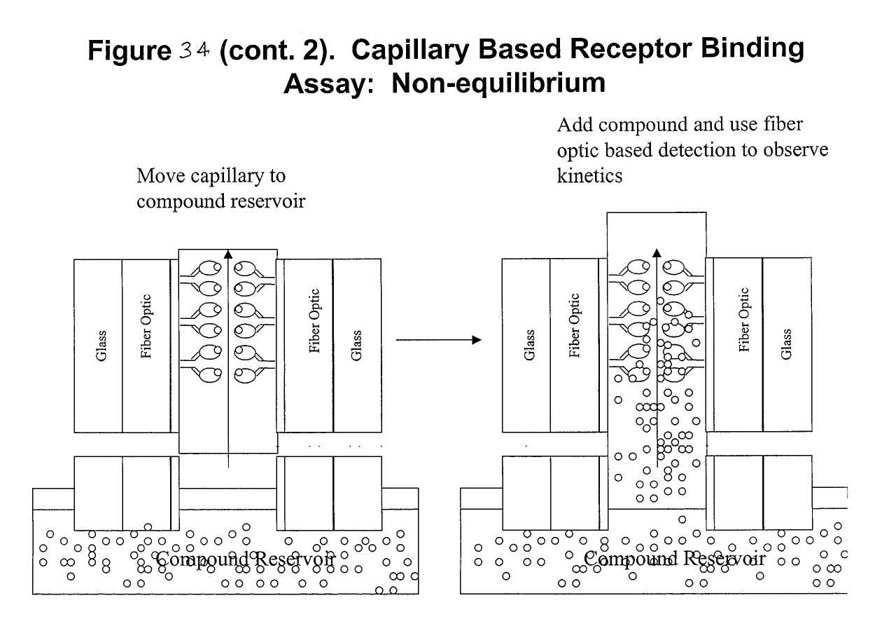

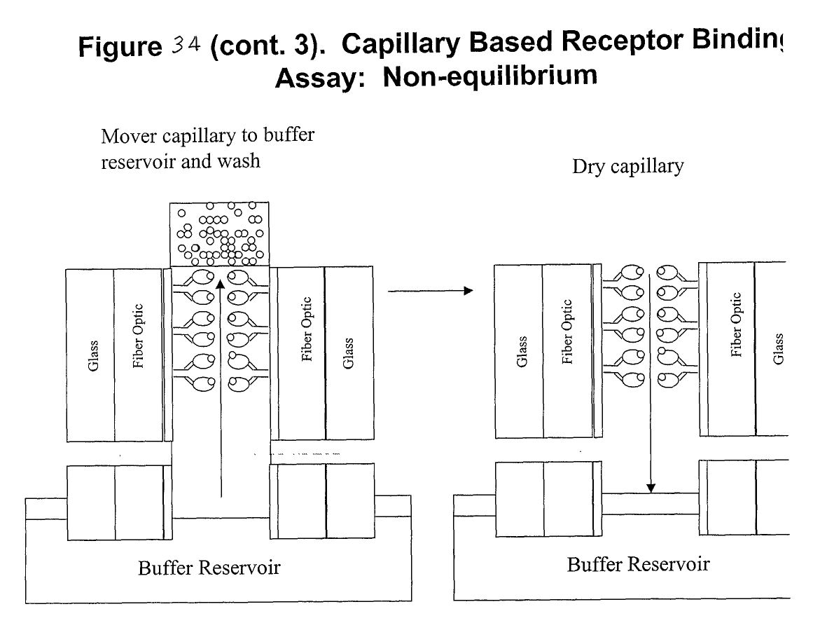

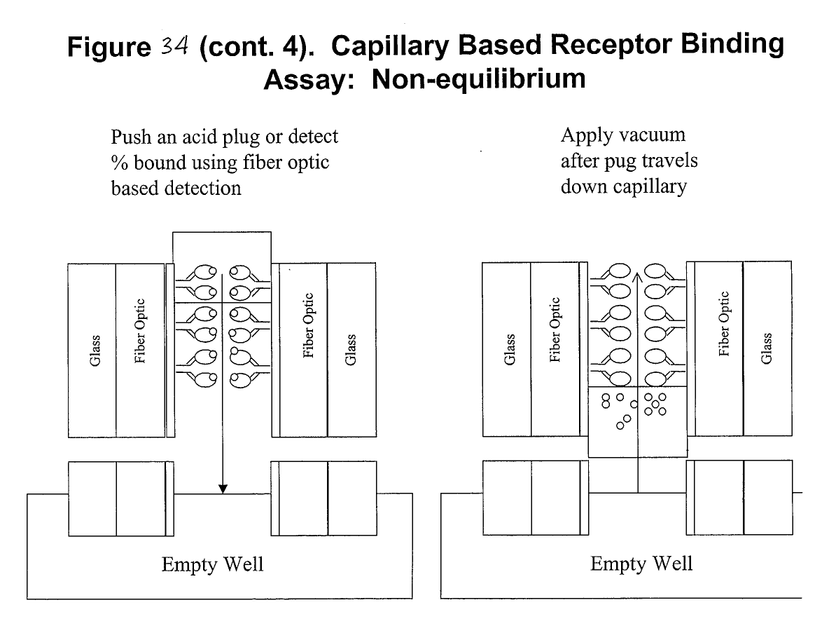

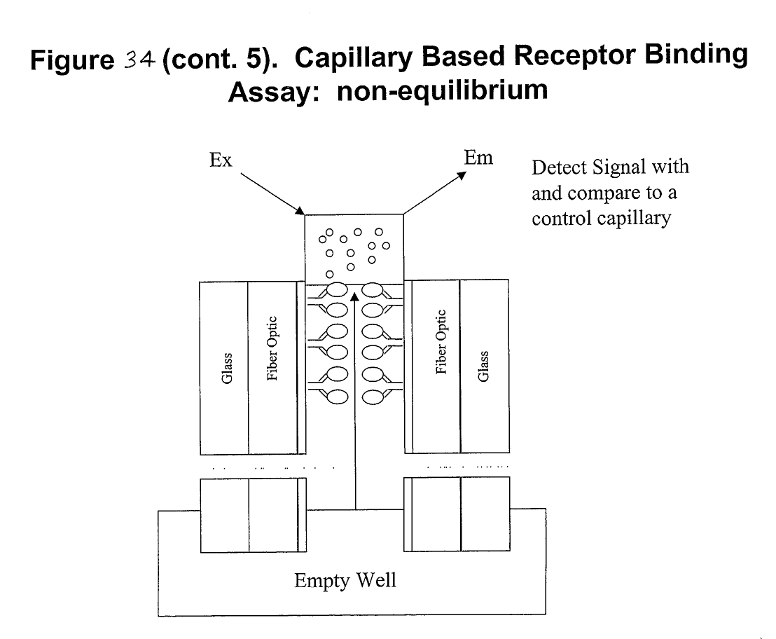

FIG. 34 schematically illustrates a non-equilibrium receptor binding assay within a library formed using fiber optic capillaries. The fiber optic portion of the capillary captures much of the signal generated and conveys the signal to a surface of the library, where a detector readily detects the signal generated in any of the capillaries of the library.

The interior wall of a capillary is silanized and coupled to an anti-receptor antibody. The receptor is then immobilized on the capillary walls by being bound to the antibodies. A saturating amount of ligand specific for the receptor is added and following incubation, unbound ligand is washed away and total bound ligand is calculated using fiber optic based detection methods. The capillary array is then transferred to a reservoir containing a compound of interest. Following addition of the compound, fiber optics based detection methods are used to follow the kinetics of competitive binding between the ligand and the compound to the receptor. The capillary array is then moved to a buffer reservoir and unbound ligand and compound are washed away. An acid plug is then introduced into the dry capillary to displace the bound ligand and compound. Once the acid plug has contacted the receptor molecules, it is extruded by a negative pressure and the signal generated by the ligand and compound mixture in the acid plug is measured against a control which was not modified by the compound. The signal may be generated by the ligand or the compound or both, but preferably the signal is generated by the ligand. When the ligand carries the fluorescence label, the detectable fluorescence decreases over time as unlabeled compound displaces the ligand. Detection of the kinetics of this process allows the avoidance of false positive data.

Receptor Binding Assay under Equilibrium Conditions

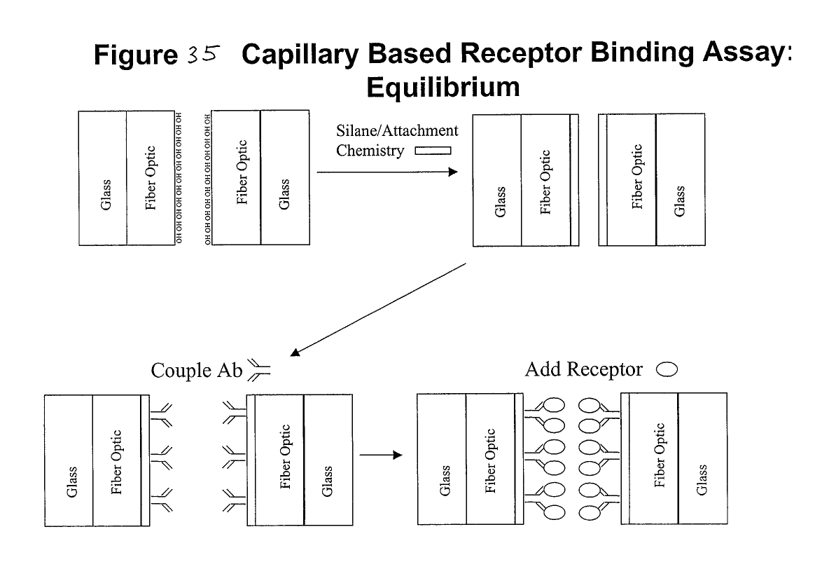

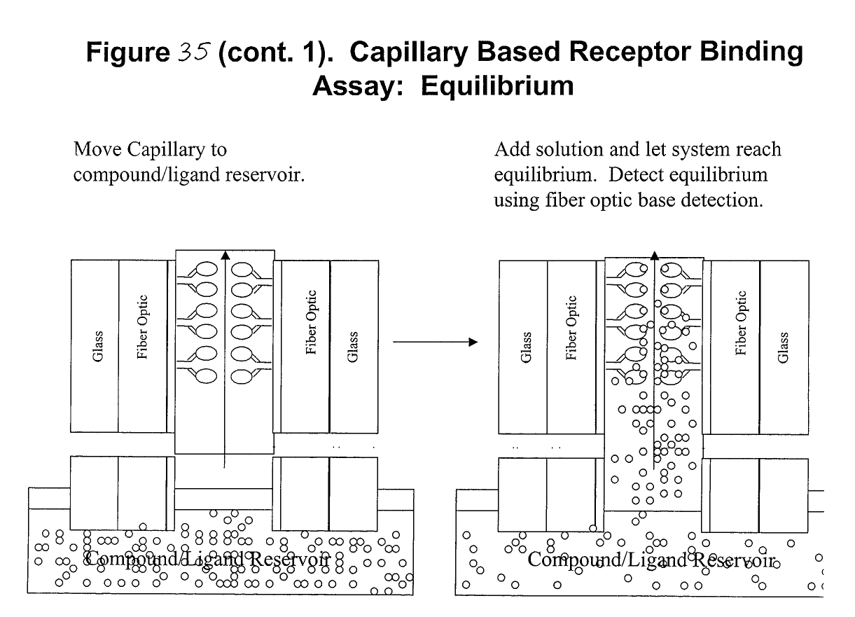

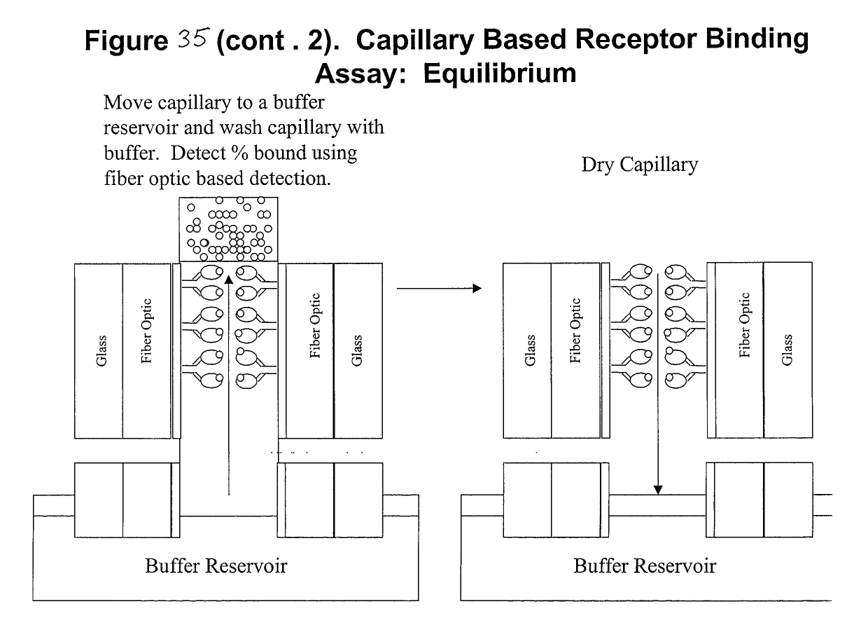

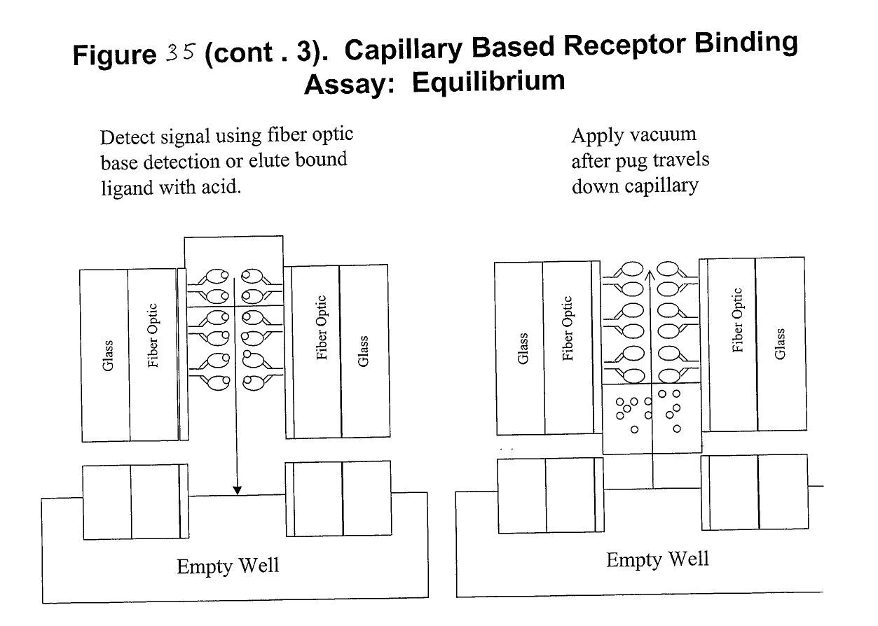

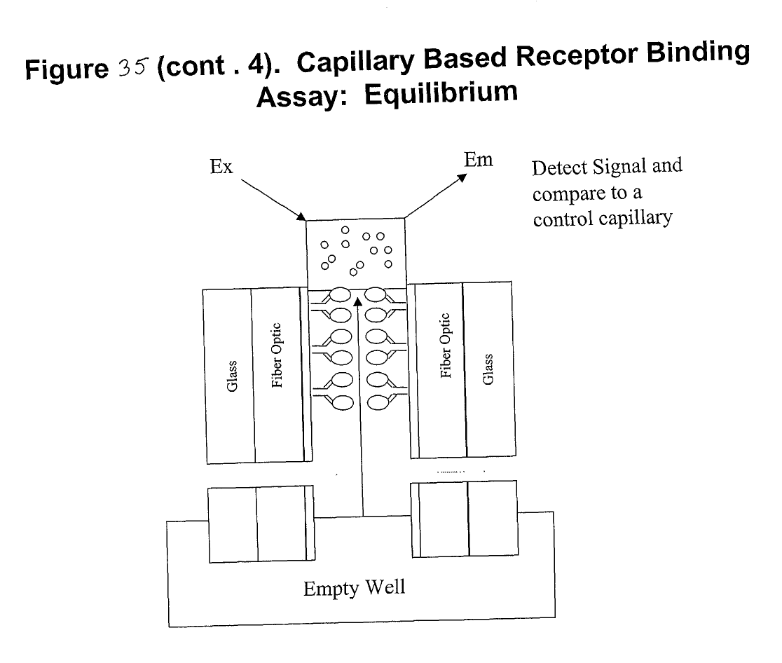

FIG. 35 schematically illustrates a receptor binding assay within a fiber optic capillary under equilibrium conditions.

The interior wall of a capillary is silanized and coupled to an anti-receptor antibody. The receptor is then immobilized on the capillary walls by being bound to the antibodies. The capillary array is transferred to a reservoir containing both ligand and compound. Sufficient ligand/compound solution is added and incubated with the receptors to reach equilibrium. The attainment of equilibrium is detected by fiber optics based detection. The capillary array is then transferred to a buffer reservoir and washed with the buffer to remove unbound ligand and compound. Percentage of ligand and/or compound bound is detected by fiber optics based detection. An acid plug is then introduced into the dry capillary to displace the bound ligand and compound. Once the acid plug has contacted the receptor molecules, it is extruded by a negative pressure and the signal generated by the ligand and compound mixture in the acid plug is measured against a control which was not modified by the compound. The signal may be generated by the ligand, or the compound or both, but preferably the signal is generated by the ligand. Since this process determines the end point of the reaction, it is not necessary to use fiber optic capillaries.

High Throughput Screening of Peptides

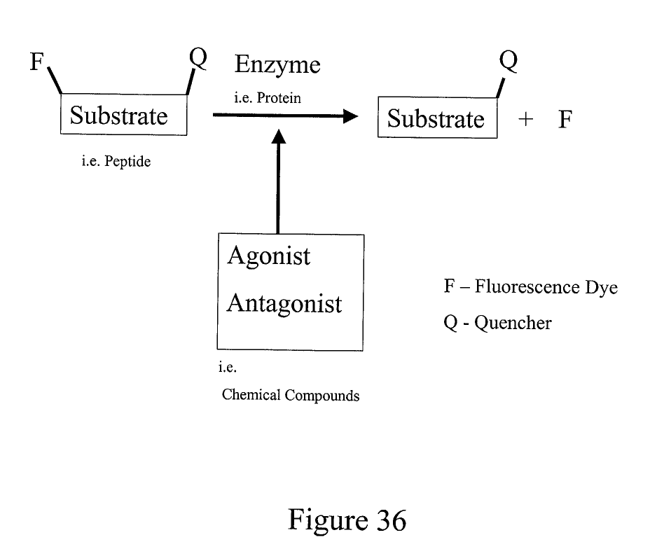

FIG. 36 illustrates an enzyme based HTS assay. As is apparent to one of ordinary skill from this figure, a substrate such as a peptide having a fluorescent moiety (F) and a quencher (Q) that quenches the fluorescent moiety is contacted with an enzyme such as protease on the surface of the reaction head. The fluorescent moiety is cleaved from the substrate by the protease, allowing the dye to fluoresce and determine the level of protease activity. When the capillary bundle delivers agonists and/or antagonists to the reaction head the amount of fluorescent moiety cleaved from the substrate is altered, allowing the determination of the agonist/antagonist activity of the test chemical compound on the enzymatic activity.

It should be readily apparent to one skilled in the art that this system can be used to assay a number of receptor-ligand interactions including interactions between proteins, nucleic acids and antibody-antigens.

A ligand according to this invention is defined as a molecule that is recognized by a particular receptor. Examples of ligands that can be investigated by this invention include, but are not restricted to, agonists and antagonists for cell membrane receptors, toxins and venoms, viral epitopes, hormones (e.g., steroids, etc.), hormone receptors, peptides, enzymes, enzyme substrates, cofactors, drugs (e.g., opiates etc.), lectins, sugars, oligonucleotides, nucleic acids, oligosaccharides, proteins, and monoclonal antibodies.

A receptor is a molecule that has an affinity for a given ligand. Receptors may be naturally-occurring or manmade molecules. Also, they can be employed in their unaltered state or as aggregates with other species. Receptors may be provided in a form attached, covalently or noncovalently, to a binding member, either directly or via a specific binding substance. Examples of receptors which can be employed in this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “ligand receptor pair” is formed when two macromolecules have combined through molecular recognition to form a complex.

Examples of receptors which can be investigated by this invention include but are not restricted to: ligands which bind to receptors, such as specific transport proteins or enzymes essential to survival of microorganisms, which are useful in a new class of antibiotics; binding site of enzymes such as the enzymes responsible for cleaving neurotransmitters and determination of ligands which bind to certain receptors to modulate the action of the enzymes which cleave the different neurotransmitters; ligandbinding sites on an antibody molecule which combines with the epitope of an antigen of interest and determining a sequence that mimics an antigenic epitope; sequences of nucleic acids synthesized to establish DNA or RNA binding sequences; catalytic polymers, preferably polypeptides, which are capable of promoting a chemical reaction involving the conversion of one or more reactants to one or more products; hormone receptors, e.g., the receptors for insulin and growth hormone and opiate receptors in the brain for determination of ligands which bind with high affinity to these receptors; determination of agonistic or antagonistic effects of chemical compounds, including peptides, on such receptor-ligand interactions are useful in the development of pharmaceutical and diagnostic assays of several compounds.

A peptide available for assay by the methods and apparatus of this invention may be defined as a polymer in which the monomers are alpha amino acids and which are joined together through amide bonds and alternatively is referred to as a polypeptide. In the context of this specification it should be appreciated that the amino acids may be the L-optical isomer or the D-optical isomer. Peptides are usually more than two amino acid monomers long. Standard abbreviations for amino acids are used according to Stryer, Biochemistry, 3d Ed., 1988, which is incorporated herein by reference for all purposes.

Cell-Based HTS Assay

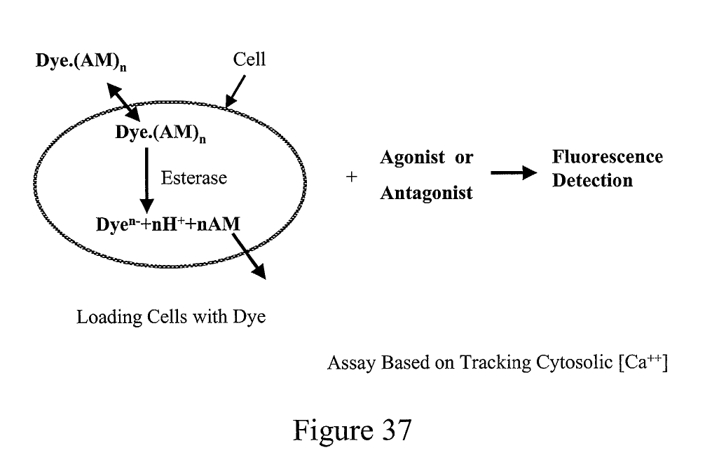

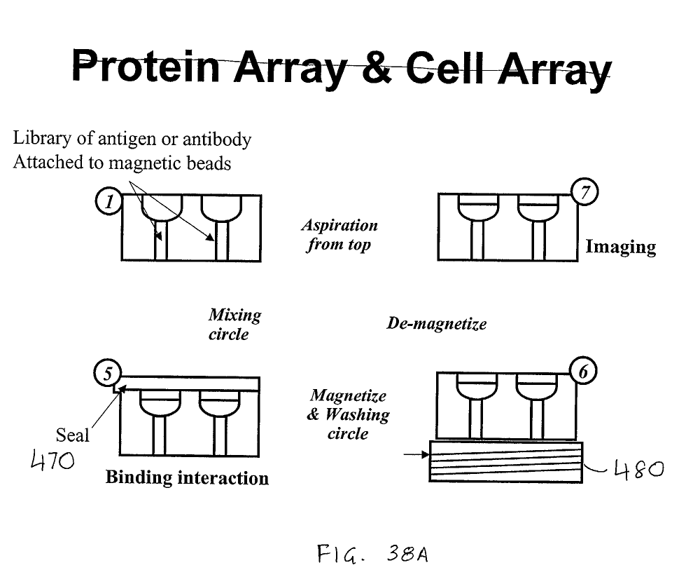

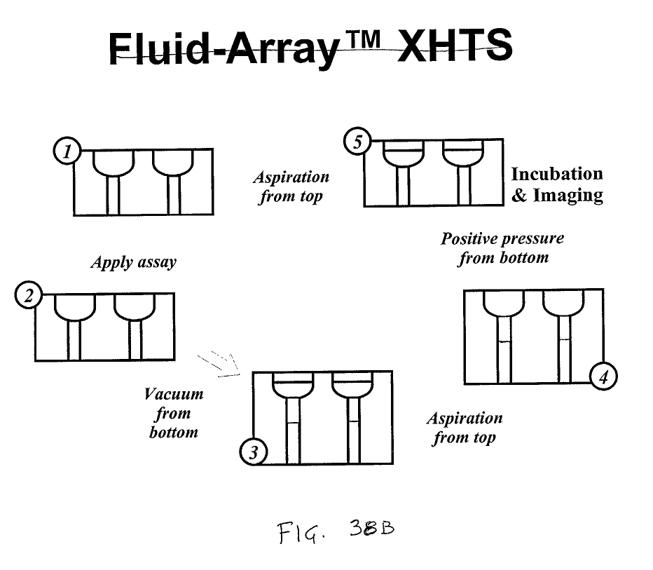

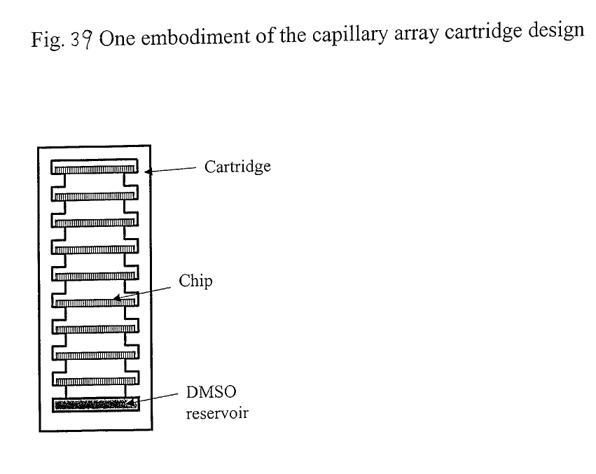

FIG. 37 illustrates an example of a cellular HTS assay based on tracking cytosolic calcium ions that can be performed using the system of the invention. As is apparent to one of ordinary skill from this figure, agonists and/or antagonists are placed in a library and delivered to the reaction head via the capillaries of the delivery or reaction head. The sample is prepared as illustrated and placed in the reaction head as described above. Once sufficient time has passed for the sample to have reacted with the agonists and/or antagonists, the reaction head is scanned using a fluorescence detector to determine the effect, if any, of the agonists and/or antagonists.

Glutamate is the major excitatory neurotransmitter in the mammalian brain. In the mammalian central nervous system (CNS) there are three main subtypes of ionotropic glutamate receptors, defined pharmacologically by the selective agonists Nmethyl-D-aspartate (NMDA), kainate (KA), and alpha-amino-3-hydroxy-5-methylisoxazole-4-propionic acid (AMPA). The NMDA receptor has been implicated in a variety of neurological pathologies including stroke, head trauma, spinal cord injury, epilepsy, anxiety, and neurodegenerative diseases such as Alzheimer's Disease (Watkins and Collingridge, The NMDA Receptor, Oxford: IRL Press, 1989). When activated by glutamate, the endogenous neurotransmitter, the NMDA receptor permits the influx of extracellular calcium and sodium ions through an associated ion channel.

Compounds active on receptor-operated calcium channels are useful for treatment of neurological disorders and diseases such as stroke, head trauma, spinal cord injury, spinal cord ischemia, ischemia- or hyproxia-induced nerve cell damage, epilepsy, anxiety, neuropsychiatric or cognitive deficits due to ischemia or hypoxia such as those that frequently occur as a consequence of cardiac surgery under cardiopulmonary bypass, or neurodegenerative diseases such as Alzheimer's Disease, Huntington's Disease, Parkinson's Disease, or amyotrophic lateral sclerosis (ALS).

ELISA—Detection of Protein-Protein Interaction Inhibitors Using Magnetic Beads

Enzyme linked immunosorption assays (ELISA) are heterogeneous assays which detect the binding between ligands in solution and immobilized receptors. ELISA requires many reagent mixing and washing steps that are difficult to perform in the 96-well format, and one could envision even greater difficulty when the wells are reduced in volume from the 96-well format to the 384-well format. ELISA is commonly used to detect inhibition of ligand-receptor interactions where the receptors are immobilized in microtitre wells. “Ligand-receptor” pairs used in ELISA can comprise any pair of binding molecules from proteins or other macromolecules to small molecules.

These assays are complex, multiple-step assays that require immobilizing the receptor, incubating receptor with ligand, washing to remove unwanted nonspecifically retained ligand that would otherwise cause high signal background, binding visualizing reagents (e.g., a ligand specific antibody conjugated to a reporter enzyme) to the receptor-bound ligand and generating a visible signal by providing substrates for the reporter enzyme. The complexity of ELISA has led the HTS industry to conclude that ELISA cannot be adapted into a free-format assay.

The invention provides methods and equipment suitable for ELISA. A receptor may be immobilized on magnetic beads as described in FIG. 38A. A ligand for the immobilized receptor is dispensed on or into the reaction chamber. Test compounds or samples are dispensed into the capillary. After a suitable incubation period, the ligand is brought into contact with the receptor, allowing the ligand and the samples to come in contact with and potentially react with the receptor by diffusion or by agitation as illustrated in FIG. 38B.

After a suitable incubation period, the receptor is removed and washed with a suitable buffer to remove unbound and non-specifically bound ligand and samples. The receptor is then contacted with a solution containing assay reagents that will interact with the ligand (e.g., an antibody, avidin or streptavidin in the case of a biotinylated ligand) and has the ability to be detected either directly (e.g., by fluorescence or radioactivity) or indirectly (e.g., horse radish peroxidase (HRP), alkaline phosphatase (AP), or β-galactosidase conjugate). After suitable incubation, the receptor is removed from the solution and washed to remove unbound and non-specifically bound reagent. In the case of direct detection the signal is imaged using the appropriate method (e.g., spectrophotometric scanners, CCD cameras, film, phosphorimagers, or scintillation detection devices). Indirect signals (e.g., HRP or AP) require an additional signal development reaction, achieved by dispensing substrates or other necessary reaction components to contact the bound receptor. The enzyme (e.g., HRP or AP) then reacts with the substrate. Under any visualization method, the ligand/receptor binding areas produce a detectable reaction, while the areas where ligand/receptor binding was inhibited do not produce a visible reaction.

Assay components include but are not limited to macromolecules such as nucleic acids, proteins, and other synthetic or natural macromolecules; cells, cell lysates, biological extracts, organelles, and other complex biological entities and mixtures; and small molecules such as buffers, salts, inhibitors, substrates, peptides, dyes, nucleotides, cofactors, ions, and solvents.

Suitable cell surface receptors include, but are not limited to, insulin receptor, insulin-like growth factor receptor, growth hormone receptor, glucose transporters (particularly GLUT 4 receptor), transferrin receptor, epidermal growth factor receptor, low density lipoprotein receptor, high density lipoprotein receptor, epidermal growth factor receptor, leptin receptor, interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor, PDGF receptor, EPO receptor, TPO receptor, ciliary neurotrophic factor receptor, prolactin receptor, and T-cell receptors. In addition, there are a number of “orphan” receptors for which biological function has not yet been fully assigned; these are referenced herein by their SwissProt reference numbers and include, but are not limited to, SwissProt ML1B (rat melatonin receptor type 1B); SwissProt SCRC (human secretin receptor precursor); SwissProt NY1R (Xenopus nueropeptide Y receptor); SwissProt PAFR (rat platelet activating factor receptor); and SwissProt BLR1 (Burkitt's Lymphoma receptor for human, mouse and rat). Algorithms for sequence analysis are known in the art, and include, but are not limited to, the Best Fit sequence program described by Devereux et al, Nucl. Acid Res. 12:387-395 (1984), with default settings preferred; BLAST, described in Altschul et al. (1990) J. Mol. Biol. 215:403-10; ADVANCE and ADAM, described in Torelli and Robotti (1994) Comput. Appl. Biosci. 10:3-5; and FASTA, described in Pearson and Lipman (1988) P.N.A.S. 85:2444-8. The sequence similarity may be determined using the Wisconsin Package, version 8.0-OpenVMS, Genetics Computer Group.

Generally, in an embodiment of the methods herein, the cell surface receptor is non-diffusably bound to the insoluble support forming a capillary or through hole. The insoluble supports may be made of any composition to which a peptide or receptor can be bound, is readily separated from soluble material, and is otherwise compatible with the overall method of screening.

The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Libraries or microarrays of the invention are especially convenient because a large number of assays can be carried out simultaneously, using small amounts of reagents and samples. The particular manner of binding of the peptide or other protein is not crucial so long as it is compatible with the reagents and overall methods of the invention, maintains the activity of the peptide and is nondiffusable. Preferred methods of binding include the use of antibodies (which do not sterically block either the ligand binding site or activation sequence when the receptor is bound to the support), direct binding to “sticky” or ionic supports, chemical crosslinking, the synthesis of the receptor on the surface, etc. Following binding of the peptide or receptor, excess unbound material is removed by washing. The sample receiving areas may then be blocked through incubation with bovine serum albumin (BSA), casein or other innocuous protein.

Use of Liquid Probes

Liquid protein probes may be made by attaching protein molecules to microscopic ferromagnetic beads that are significantly smaller that the diameter of the through holes in the array. These beads are further suspended in a suitable buffer fluid. In another embodiment, the proteins are dissolved in solution phase.

Within each through hole, the liquid probe may be either homogeneous or heterogeneous. In the latter case, the probe may comprise different solutions or the same solution of different concentrations. These difference elements of the probe are distributed in different sections along the through hole and may be separated by an air gap, as illustrated in FIG. 39.

A protein probe can be dissolved in solution and not attached to beads. In addition, the inner surface of the microwell can be coated with an agent such as an antibody or avidin/streptavidin that binds or has a high affinity to the protein molecule. Such treated surface can immobilize the protein probes once they get inside the wells. Thereafter, washing can be conducted in the wells to remove nonbinding molecules from the wells.

In one embodiment, the through holes are 20 μm in diameter with a pitch of 100 μm across the array. The reaction wells are 80 μm in diameter and 80 μm deep, providing a 0.4 nl volume. One example of a liquid array has 100,000 holes, and the array itself is about 30 mm in diameter. The volume of each probe used in one experiment is less than 0.4 nl in this device. The volume of target liquid used in one experiment is therefore less than 40 μl.

As mentioned above, each through hole in the array may contain more than a single homogenous solution, separated by a small air gap. In this case, a sequence of solutions can be pumped into the microwell for the reaction. This enables the liquid protein array to perform much more complex assays.

The branch format for a library is especially suitable for continuous and repeated use for a long period of time. The bundle format can be used repeatedly for a large number of times with the length of the capillary bundle determining the number of repeated usages. Once the bundle is emptied, it is typically discarded. The chip format is designed for a single or a small number of usages. Assuming 20 μm in diameter, a through hole of 10 mm in length can hold 3 nl of liquid probe, which is sufficient for approximately 6 experiments. As described above, it is possible to build a fluid reservoir at the proximal end of each through hole by enlarging the inner diameter of the hole. Assuming the through hole in the above case has a 6 mm section with an enlarged inner diameter of 80 μm at the proximal end, as shown in FIG. 2B, the probe volume held in the through hole would be increased to 30 nl, sufficient for about 60 experiments.

Suitable Probes and Target Molecules

The probes can be anything that is fit to be stored in solution and transported by through holes, including, without limitation, deoxyribonucleic acids (DNA), ribonucleic acids (RNA), synthetic oligonucleotides, antibodies, proteins, peptides, lectins, modified polysaccharides, synthetic composite macromolecules, functionalized nanostructures, synthetic polymers, modified/blocked nucleotides/nucleosides, modified/blocked amino acids, fluorophores, chromophores, ligands, chelates, haptens and drug compounds. Preferably, the probes are polypeptides.

In particular embodiments, the biological target molecule is a polypeptide, a nucleic acid, a carbohydrate, a nucleoprotein, a glycopeptide or a glycolipid, preferably a polypeptide, which may be, for example, an enzyme, a hormone, a transcription factor, a receptor, a ligand for a receptor, a growth factor, an immunoglobulin, a steroid receptor, a nuclear protein, a signal transduction component, an allosteric enzyme regulator, and the like. The target molecule may comprise the chemically reactive group without prior modification of the target molecule or may be modified to comprise the chemically reactive group, for example, when a compound comprising the chemically reactive group is bound to the target molecule.

Other embodiments of the above described methods employ libraries of organic compounds which comprise aldehydes, ketones, oximes, hydrazones, semicarbazones, carbazides, primary amines, secondary amines, tertiary amines, Nsubstituted hydrazines, hydrazides, alcohols, ethers, thiols, thioethers, thioesters, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds and/or acid chlorides, preferably aldehydes, ketones, primary amines, secondary amines, alcohols, thioesters, disulfides, carboxylic acids, acetals, anilines, diols, amino alcohols and/or epoxides, most preferably aldehydes, ketones, primary amines, secondary amines and/or disulfides.

Biological target molecules that find use in the present invention include all biological molecules to which a small organic molecule may bind and preferably include, for example, polypeptides, nucleic acids, including both DNA and RNA, carbohydrates, nucleoproteins, glycoproteins, glycolipids, and the like. The biological target molecules that find use herein may be obtained in a variety of ways, including but not limited to commercially, synthetically, recombinantly, from purification from a natural source of the biological target molecule, etc.

In a particularly preferred embodiment, the biological target molecule is a polypeptide. Polypeptides that find use herein as targets for binding to organic molecule ligands include virtually any peptide or protein that comprises two or more amino acids and which possesses or is capable of being modified to possess a chemically reactive group for binding to a small organic molecule. Polypeptides of interest finding use herein may be obtained commercially, recombinantly, synthetically, by purification from a natural source, or otherwise and, for the most part are proteins, particularly proteins associated with a specific human disease condition, such as cell surface and soluble receptor proteins, such as lymphocyte cell surface receptors, enzymes, such as proteases and thymidylate synthetase, steroid receptors, nuclear proteins, allosteric enzyme inhibitors, clotting factors, serine/threonine kinases and dephosphorylases, threonine kinases and dephosphorylases, bacterial enzymes, fungal enzymes and viral enzymes, signal transduction molecules, transcription factors, proteins associated with DNA and/or RNA synthesis or degradation, immunoglobulins, hormones, receptors for various cytokines including, for example, erythropoietin/EPO, granulocyte colony stimulating receptor, granulocyte macrophage colony stimulating receptor thrombopoietin (TPO), IL-2, IL-3, IL-4, IL-5, IL-6, IL-10, IL-11, IL-12, growth hormone, prolactin, human placental lactogen (LPL), CNTF, octostatin, various chemokines and their receptors such as RANTES, (regulated upon activation, normal T cell expressed and secreted MIP1-.alpha., IL-8, various ligands and receptors for tyrosine kinase such as insulin, insulinlike growth factor 1 (IGF-1), epidermal growth factor (EGF), heregulin-.alpha. and heregulin-.beta., vascular endqthelial growth factor (VEGF), placental growth factor (PLGF), tissue growth factors (TGF-.alpha. and TGF-.beta.), other hormones and receptors such as bone morphogenic factors, follicle stimulating hormone (FSH), and leutinizing hormone (LH), tissue necrosis factor (TNF), apoptosis factor-1 and -2 (AP-1 and AP-2), mdm2, and proteins and receptors that share 20% or more sequence identity to these.

The biological target molecule of interest will be chosen such that it possesses or is modified to possess a chemically reactive group which is capable of forming a covalent bond with members of a library of small organic molecules. For example, many biological target molecules naturally possess chemically reactive groups (for example, amine groups, thiol groups, aldehyde groups, ketone groups, alcohol groups and a host of other chemically reactive groups; see below) to which members of an organic molecule library may interact and covalently bond. In this regard, it is noted that polypeptides often have amino acids with chemically reactive side chains (e.g., cysteine, lysine, arginine, and the like). Additionally, synthetic technology presently allows the synthesis of biological target molecules using, for example, automated peptide or nucleic acid synthesizers, which possess chemically reactive groups at predetermined sites of interest. As such, a chemically reactive group may be synthetically introduced into the biological target molecule during automated synthesis.

Moreover, techniques well known in the art are available for modifying biological target molecules such that they possess a chemically reactive group at a site of interest which is capable of forming a covalent bond with a small organic molecule. In this regard, different biological molecules may be chemically modified (using a variety of commercially or otherwise available chemical reagents) or otherwise coupled, either covalently or non-covalently, to a compound that comprises both a group capable of linking to a site on the target molecule and a chemically reactive group such that the modified biological target molecule now possesses an available chemically reactive group at a site of interest. With regard to the latter, techniques for linking a compound comprising a chemically reactive group to a target biomolecule are well known in the art and may be routinely employed herein to obtain a modified biological target molecule which comprises a chemically reactive group at a site of interest.

Advantages of the Invention

A system according to this invention has substantial advantages over existing systems. A system of the invention having e.g., 5×10⁵ fiber capillary tubes at a density of 10,000 wells per cm can process approximately 1,200,000,000 data points per day (in about 8 hrs) using a substantially reduced reagent volume of about 0.2nl and resulting in significant cost savings in reading each data point.

Use of capillaries for conducting reactions allows very small reaction volumes while minimizing distortions caused by evaporation. The methods of the invention are applicable to any combination of probe and substrate and is thus suitable for any array based method including quantitative PCR, DNA sequencing, etc. Further, the use of capillaries allows use of liquid-phase reactions which avoid denaturation of proteins and cells and allow direct assay for agonists and antagonists on proteins and cells.

The methods of the invention also allow combination of primary and secondary screens in the same test to eliminate false positive signals, for example, when the secondary screen is set at different dilution levels.

The XHTS (extreme high throughput screening) system can also be adapted to screen for insecticides or fingicides. A bacterial or fungal lawn grown in agar can be layered on top of the XHTS chip. The compounds in each well of the chip will slowly diffuse into the agar layer. If a compound exhibits insecticidal or fingicidal activity, then a clearing spot will exist in the agar layer right on top of the compound well. By comparing the size of the clearing spot, the activity/potency of the compound can be measured. It is also possible to conduct enzyme or cell-based high throughput screening assays in agarose media.

IV. High Throughput Screening of Enzymes

FIG. 36 illustrates an enzyme based HTS assay. In its simplest format, such an assay utilizes a non-fluorescent molecule as the substrate for the target enzyme of interest. The target enzyme can be a protease, a phosphatase, a transferase, an oxidoreductase etc. During the course of the enzymatic reaction, the non-fluorescent substrate is enzymatically converted to another molecule possessing fluorescent properties allowing it to be detected by measurements of the fluorescent intensity of the reaction chamber. Examples of such non-fluorescent substrates that can be enzymatically converted into fluorescent products are the amino-methyl coumarin (AMC) based peptides, all of which have an AMC residue attached to their C-terminal ends. In the intact form, the substrates are non-fluorescent but proteolytic cleavage releases the highly fluorescent molecule AMC which can be detected. Alternatively, proteases can be assayed using a different class of fluorogenic substrates, which are based on the phenomenon of fluorescence resonance energy transfer (FRET). A typical substrate of this sort is a peptide that is modified by two chemical entities, one a fluorescent molecule (sometimes called a donor) and the other a molecule that quenches the emitted fluorescence of the first moiety (sometimes called a quencher or acceptor). A typical, widely used donor acceptor pair is the EDANS-DABCYL pair, where EDANS is the fluorophore whose fluorescence emission is efficiently quenched by the DABCYL in the intact form of the peptide substrate molecule. Upon proteolytic cleavage, the two molecules are separated by the cleavage of a peptide bond, and the fluorescence of EDANS can be easily detected. In the case of other enzymes, such as phosphatases, similar fluorogenic substrates are also known. They can be based on relatively simple fluorescent molecules, such as coumarin, fluorescein and others. The phosphate derivatives of these molecules, such as coumarin phosphate (also known as umbelliferyl phosphate) and fluorescein diphosphate are non-fluorescent molecules, which are efficient substrates for numerous target phosphatases. The enzymatic dephosphorylation (i.e., the removal of the phosphate groups from the substrates) generates highly fluorescent products. Another class of enzymes that can be assayed in a similar fluorogenic format are all enzymes utilizing NADH/NAD or NADPH/NADP as cofactors. Such enzymes typically belong to the oxido-reductases. In some cases, if a certain enzymatic reaction cannot be directly assayed with a suitable fluorogenic substrate, it may be coupled with another reaction, involving a second enzyme, that uses one of the products generated in the first reaction and converts this into another molecule whereby the process is accompanied by the generation of a fluorescent molecule. As an example, a protein kinase can be assayed by taking advantage of one of the products of the enzymatic reaction, namely adenosine diphosphate (ADP). This product can be used as the substrate for a second enzyme, pyruvate kinase. In the presence of phosphoenol pyruvate, the ADP will be phosphorylated by the pyruvate kinase to ATP, with the concomitant generation of pyruvate. The latter can be reduced enzymatically by a third enzyme present, lactate dehydrogenase. The lactate dehydrogenase-catalyzed reaction utilizes NADH as the reducing reagent, which in the course of the reaction is converted to NAD. Since the fluorescent properties of NAD and NADH are significantly different, the reaction progress can be monitored by measuring the fluorescence intensity.

Often, suitable fluorogenic substrates are not available to assay certain enzyme targets. For example, the enzymatic phosphorylation of peptides or proteins by protein kinases cannot, in general, be directly detected by a change in the fluorescence intensity, even if a fluorescent tag is attached to the substrate. In cases like these, other approaches can be used that take advantage of the different chemical and physical properties of the non-phosphorylated substrate and phosphorylated product molecules. For example, antibodies can be generated that recognize specifically the phosphorylated reaction products. The enzymatic phosphorylation reaction can therefore be followed by methods such as fluorescence polarization (FP) and homogeneous time resolved fluorescence (HTRF). The fluorescence polarization assays can be carried out as direct assays, where the peptide substrate is labeled with a fluorophore and the binding of the antibody to the generated phosphorylated product is detected by an increase in the FP signal. In a preferred format, the assay is carried out in a competitive format, using an unlabeled peptide substrate and a fluorescently labeled phosphoprotein tracer. At the beginning of the assay the antibody binds the labeled tracer and the FP signal is high. As the enzymatic reaction progresses, increasing concentrations of unlabeled phosphorylated product are generated that compete with the labeld tracer for antibody binding. As a consequence, the FP signal decreases.

Alternative assay format that utilizes an array of compounds on a flat surface

An alternative format for high-throughput screening which could utilize some of the advantages of the capillary arrays is a format whereby the compounds to be tested are delivered via the capillary bundles onto a flat, solid surface. The compounds can be delivered as solutions in pure DMSO, or any other suitable solvent, and the volumes of the individual droplets delivered will be determined by the dimensions of the capillaries, the applied pressure and the duration of the application of pressure.

Following the delivery of the compounds, the drops can be allowed to dry and the compounds will thus form an array on the surface, whereby the density and the distances between the individual compounds will be determined by the parameters of the delivering capillary bundle. The array of dry compounds thus generated can be stored for extended periods of time, preferably under dry and cold conditions to prevent compound extended periods of time, preferably under dry and cold conditions to prevent compound degradation. For high throughput screening, the array of compounds can be overlaid with a layer of a suitable polymer, such as agarose, polyacrylamide etc. which can also contain the enzyme target of interest. The main purpose of this polymer is to decrease the rate of diffusion of the compounds away from the position where they were initially deposited and allow the effect of every compound to be localized. In addition, the presence of such a polymer reduces the rate of evaporation. The application of the polymer can be done by spin-coating, dip-coating or simply by pipetting the solution of the polymer and allowing it to polymerize or solidify over the array of compounds. Following this step, the deposited compounds will start to re-dissolve and diffuse into the polymer layer. Those compounds that have an affinity for the target enzyme will bind to it. Finally, a suitable substrate for the enzyme can be delivered to the system by either overlaying the polymer with the embedded enzyme with another layer of polymer containing the substrate, or by simply pipetting the substrate solution onto the polymer and allowing the solution to penetrate the polymer. The reaction of enzyme and substrate will generate a reaction product, whose formation can be detected by, for example, measuring the fluorescent signal in the case of a fluorogenic assay. If some of the deposited compounds act as inhibitors of the target enzyme, the regions over the spots will generate less product than similar regions over inert compounds.

All publications and patent applications cited in this specification are herein incorporated by reference in their entirety as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A high throughput screening apparatus for ultra high throughput screening of chemical compounds for a biological target, comprising a substrate having a plurality of channels, wherein each of said channels has a first end at a first face of the apparatus and wherein each of said channels has a second end, each said first end being one of a plurality of first ends and each said second end being one of a plurality of second ends, wherein the first ends of said channels are secured to a plurality of reaction chambers such that at least one of said first ends is in fluid communication with a different reaction chamber from at least one other of said first ends.

2. A high throughput screening apparatus according to claim 1, wherein said reaction chamber forms a portion of said first end.

3. A high throughput screening apparatus according to any of claims 1-2 wherein said reaction chamber is defined by a hydrophilic region at the first face of said apparatus surrounded by a hydrophobic region at the first face of said apparatus.

4. A high throughput screening apparatus according to claim 1 wherein each said reaction chamber is defined by a hydrophilic region at the first end of the channel corresponding to the reaction chamber and is further defined by a hydrophobic region surrounding the hydrophilic region at the first end of the channel.

5. A high throughput screening apparatus according to claim 1, wherein each of said first ends has an individual reaction well forming said reaction chamber, the reaction well having a cross-sectional area that is greater than a cross-sectional area of its corresponding channel.

6. A high throughput screening apparatus according to claim 5, wherein said reaction well has an inner cross-sectional area and said channel has an inner cross-sectional area and wherein the inner cross-sectional area of said reaction well is no less than 5 times larger than the inner cross-sectional area of said channel.

7. A high throughput screening apparatus according to any of claims 5-6 wherein said reaction well is defined by a hydrophilic region on the first face of said apparatus surrounded by a hydrophobic region on said first face.

8. A high throughput screening apparatus according to any of claims 1-7, wherein each said second end is individually positionable.

9. A high throughput screening apparatus according to claim 8, wherein each said second end is individually attached to a corresponding through-channel of a plate configured to fit a microtiter plate such that each through-channel of the plate corresponds to an individual well of the microtiter plate.

10. A high throughput screening apparatus according to any of claims 1-7, wherein each said second end is positioned at a second face of the apparatus.

11. A high throughput screening apparatus according to claim 10 wherein the channels have a volume such that only one assay is performed using the apparatus.

12. A high throughput screening apparatus according to claim 10 wherein said apparatus has a first metering section and a second metering section, the first metering section comprising a channel of said channels wherein said channel is configured to draw a desired volume of a target liquid into the channel by capillary force, and the second metering section comprising a well on said second surface, the well being formed by a hydrophilic area and a hydrophobic area placed around an entrance to the channel such that a droplet of a second liquid of a desired volume forms when the second liquid is placed on said second surface.

13. A high throughput screening apparatus according to claim 10 wherein the apparatus has a length less than 50 cm.

14. A high throughput screening apparatus according to claim 13 wherein the apparatus has a length less than 10 cm.

15. A high throughput screening apparatus according to claim 13 wherein the apparatus has a length less than 1 cm.

16. A high throughput screening apparatus according to any of claims 10-15 wherein said channels have hydrophobic portions along a length of each of the channels such that fluid entering the channels by capillary action stops at the hydrophobic portions.

17. A high throughput screening apparatus according to any of claims 10-16 wherein each of said channels has a portion having a length and a cross-sectional area selected to provide a desired amount of a fluid when said fluid enters said channels through capillary action.

18. A high throughput screening apparatus according to any of claims 10-17 wherein the reaction chamber is lined with a reflective material.

19. A high throughput screening apparatus according to any of claims 10-18 wherein each second end forms a well having a cross-sectional area greater than a cross-sectional area of the channel.

20. A high throughput screening apparatus according to any of claims 1- 19 and further comprising a first removable sealant upon said first face.

21. A high throughput screening apparatus according to any of claims 1-20, wherein the apparatus has at least about 500 channels per cm2 as measured at the first ends of the channels.

22. A high throughput screening apparatus according to any of claims 1-21, wherein each channel has a wall that is sufficiently hydrophilic and an inner diameter that is sufficiently small such that a liquid is retained within the channel by capillary force.

23. A high throughput screening apparatus according to any of claims 1-22, wherein the high throughput screening apparatus has at least 100 channels.

24. A high throughput screening apparatus according to claim 23, wherein the high throughput screening apparatus has at least 500 channels.

25. A high throughput screening apparatus according to any of claims 1-23, wherein said first ends are secured to one another in a solid mass such that the first ends are substantially coplanar in a static array in a facet of the solid mass.

26. A high throughput screening apparatus according to any of claims 1-25, wherein said apparatus is formed of a bundle of a plurality of capillaries.

27. A high throughput screening apparatus according to claim 26, wherein said capillaries comprise light-conducting capillaries.

28. A high throughput screening apparatus according to any of claims 1-27, wherein each of said channels contains a distinct probe in solution.

29. A high throughput screening apparatus according to claims 1-27, wherein each of said channels contains a distinct probe attached to a wall of each of said channels.

30. A high throughput screening apparatus according to any of claims 28-29, wherein each of said probes is selected from a group consisting of deoxyribonucleic acids (DNA), ribonucleic acids (RNA), synthetic oligonucleotides, antibodies, proteins, peptides, lectins, modified polysaccharides, synthetic composite macromolecules, functionalized nanostructures, synthetic polymers, modified/blocked nucleotides/nucleosides, modified/blocked amino acids, fluorophores, chromophores, ligands, chelates, haptens and drug compounds.

31. A high throughput screening apparatus according to claim 30, wherein the probe comprises a tyrosine kinase protein selected from the group consisting of epidermal growth factor receptor (EGFR), platelet-derived growth factor receptor (PDGFR), fibroblast growth factor receptor (FGFR), colony stimulating factor-1, (CSF-1), insulin receptor, phospholipase C-□ (PLC-□) and insulin like growth factor-1 (IGF-1)

32. A high throughput screening apparatus according to any of claims 28-31, wherein said probe is coupled to a bead.

33. A high throughput screening apparatus according to any of claims 28-31, wherein the probe is in a solution phase.

34. A high throughput screening apparatus according to any of claims 28-31, wherein the probe is in a colloidal dispersion.

35. A system for providing a combinatorial library, said system comprising a high throughput screening apparatus according to any of claims 1-27 and a fluid delivery system comprising a filling tube having a size suitable to fill a channel of the high throughput screening apparatus.

36. A system for providing a combinatorial library, said system comprising a high throughput screening apparatus according to any of claims 1-7 and 10-27 and a fluid delivery system comprising a plurality of capillaries having first ends positioned so that said first ends match with the second ends of the channels of the high throughput screening apparatus.

37. A system according to claim 36, wherein the first ends of the capillaries of the delivery system are in a capillary face that fits against the first face of the high throughput screening apparatus.

38. A desktop screening system comprising a system according to any of claims 35-37 and a detector that detects a signal indicative of an interaction between a probe and a target.

39. A method of fabricating a high throughput screening apparatus comprising
(a) forming a substrate having channels that terminate in a first face; and
(b) forming a plurality of reaction chambers at the first face such that at least one of said channels is in fluid communication with a different reaction chamber from at least one other of said channels.

40. A method according to claim 39 wherein the act of forming a plurality of reaction chambers provides that each of the channels terminates individually at a reaction chamber of said plurality.

41. A method according to any of claims 39-40 wherein said reaction chamber comprises a virtual well formed by a hydrophilic region on the first face immediately adjacent to an individual channel, which hydrophilic region is surrounded by a hydrophobic region.

42. A method according to claim 41 wherein said virtual well is formed by applying a mask to the first face and exposing an unmasked portion of the first face to energy sufficient to cleave a cleavable portion of a molecule attached to the first face.

43. A method according to any of claims 39-42 wherein the act of forming a substrate having said channels comprises forming a bundle of capillaries so that said channels of the capillaries terminate in said first face.

44. A method according to claim 43 and further comprising forming said bundle of capillaries such that said channels of the capillaries terminate in a second face.

45. A method according to claim 44 and further comprising forming a virtual well around each channel at said second face by providing a hydrophilic region on the second face immediately adjacent to said channel and surrounding said hydrophilic region with a hydrophobic region.

46. A method according to claim 45 wherein said virtual well is formed by applying a mask to the first face and exposing an unmasked portion of the first face to energy sufficient to cleave a cleavable portion of a molecule attached to the first face.

47. A method according to claim 44 and further comprising forming a physical well at each channel at said second face by providing an opening at said second face having a cross-sectional area greater than a cross-sectional area of the channel.

48. A method according to claim 47 wherein said physical well is formed by etching the substrate.

49. A method for fabricating an array according to claim 3 or claim 7, the method comprising applying a mask to define the hydrophilic zone and the hydrophobic zone.

50. A method of metering a volume of a liquid comprising placing a first liquid having a surface tension suitable to form a droplet of predetermined volume in a hydrophilic region and a hydrophobic region of a substrate, which hydrophobic region surrounds said hydrophilic region, and removing the first liquid from the hydrophobic region.

51. A method according to claim 50 wherein the substrate has a channel that opens to the hydrophilic region of the substrate, and wherein the channel has been filled with a second liquid prior to placing the first liquid in the hydrophilic region and the hydrophobic region.

52. A method according to claim 51 wherein the channel has a cross-sectional area and length to provide a predetermined volume of the second liquid within the capillary.

53. A method according to claim 52 wherein the method further comprises moving both the first liquid and the second liquid through the channel and into a reaction chamber.

54. A method according to claim 53 wherein the reaction chamber forms a portion of the substrate.

55. A method for determining an affinity of a compound for a receptor, the method comprising:
(a) immobilizing the receptor on a wall defining a channel of an array of channels;
(b) binding to the receptor a saturating amount of ligand specific for the receptor;
(c) contacting the array with an excess of said compound; and
(d) measuring a rate of displacement of bound ligand by the compound.

56. A method according to claim 55 wherein the array of channels comprises a capillary array, and the wall defining said channel comprises a capillary.

57. A method according to claim 56 wherein the capillary is an optical fiber capillary and the act of measuring the rate of displacement comprises measuring the rate of displacement of bound ligand by the compound by monitoring an intensity of light conveyed by the optical fiber capillary.

58. A method according to any of claims 55-57, further comprising:
(a) removing unbound ligand and compound from the channel;
(b) introducing an acid plug into the channel to elute all bound ligand and compound into the acid plug; and
(c) measuring a signal indicative of the ligand and compound, wherein at least one of the ligand and the compound is coupled to a moiety that produces a detectable signal.

59. A method for determining an affinity of a compound for a receptor, the method comprising:
(a) immobilizing the receptor on a wall defining a channel of an array of channels;
(b) binding to the receptor a mixture of a saturating amount of a ligand specific for the receptor and a saturating amount of a compound; and
(c) measuring a ratio of bound ligand to bound compound.

60. A method according to claim 59, wherein the array of channels comprises a capillary array, and the wall defining said channel comprises a capillary.

61. A method according to claim 60 wherein the capillary is an optical fiber capillary and the method further comprises measuring the ratio of bound ligand to bound compound by monitoring light conveyed by the optical fiber capillary.

62. A method according to any of claims 59-61, further comprising:
(a) removing unbound ligand and compound from the channel;
(b) introducing an acid plug into the channel to elute all bound ligand and compound into the acid plug; and
(c) measuring a signal indicative of the ligand and compound, wherein at least one of the ligand and the compound is coupled to a moiety that produces a detectable signal.
